(19) United States
(12) Patent Application Publication
(10) Pub. No.: US 2022/0354098 A1
(43) Pub. Date: Nov. 10, 2022

(54) Genetically Modified Non-Human Animals and Methods of Use Thereof

(71) Applicant: YALE UNIVERSITY, New Haven, CT (US)

(72) Inventors: Richard Flavell, Guilford, CT (US); Stephanie Halene, New Haven, CT (US); Yuanbin Song, Guangzhou (CN)

(21) Appl. No.: 17/626,903

(22) PCT Filed: Jul. 17, 2020
(86) PCT No.: PCT/US20/42475

§ 371 Date: Jan. 13, 2022

Related U.S. Application Data
(60) Provisional application No. 62/875,108, filed on Jul. 17, 2019.

(51) Int. Cl. A01K 67/027

(52) U.S. Cl. CPC A01K 67/0278; A01K 67/0271; A01K 2267/0387; A01K 2217/072; A01K 2227/105; A01K 2267/0306; A01K 2267/0381; A01K 2267/01; A01K 2207/12

(57) ABSTRACT

The invention relates generally to genetically modified non-human animals expressing human polypeptides and their methods of use,

### CROSS-REFERENCE TO RELATED APPLICATIONS

This application claims the benefit of U.S. Provisional Application No. 62/875,108, filed Jul. 17, 2019, the disclosure of which is hereby incorporated by reference in its entirety,

### BACKGROUND OF THE INVENTION

Sickle Cell Disease (SOD) is caused by a single point mutation in the JI-globin gene mutating the 6^{th} AA glutamine to valine in HgS or a lysine in HgC. SCD occurs in the homozygous or compound heterozygous state or when HgS mutations in one allele are coupled to β-thalassemia mutations on the second allele. Symptoms of SCD typically begin around 5 to 6 months of age when fetal γ-globin switches almost entirely to adult β-globin synthesis. Red blood cell (RBC) sickling is caused by the loss of red blood cell elasticity due to repetitive polymerization of the mutant hemoglobin under low oxygen tension. Red blood cell sickling is thus most pronounced in the periphery and When oxygen delivery is limited due to vaso-occlusion by sickled cells. Signs of SCD include, among others, hemolysis and anemia, vase-occlusive crises (VOC) accompanied by tissue hypoxia, splenic sequestration and progressive splenic auto-infarction, acute chest syndrome, and chronic organ damage, Extensive studies have identified numerous contributing mechanisms to the severity of SCD that range from genetic factors (such as the remaining degree of γ-globin expression) to infection and inflammation. The most used murine models of SCD (Nagel RL et al., 2001, Br J Haematol., 112:19-25) express exclusively human globins in the background of murine globin knockouts (Paszty C et al., 1997, Science, 278:876-878; Ryan TM et al., 1997, Science, 278:873-876; Chang JC et al., 1998, Proc Natl Acad Sci USA, 95:14886-14890). Mouse models have significantly contributed to elucidation of many of the mechanisms of SCD pathology but exclusively contain mouse red cells and fail to capture the heterogeneity encountered in patients. Not. one model is sufficient requiring careful choices to interpret findings and in particular the effect of genetic modifications (such as modulation of human γ-globin expression) and other therapeutics.

Thus, there is a need in the art for humanized non-human animals able to support and sustain engraftment with human hematopoietic cells (e.g., red blood cells). The present invention addresses this unmet need in the art.

### SUMMARY OF THE INVENTION

In one aspect, the present invention provides a genetically modified non-human animal comprising: a) a genome comprising a nucleic acid encoding at least one of the group consisting of human M-CSF, human IL-3, human GM-CSF, human SIRPA, and. human TPO, wherein the nucleic acid is operably linked to a promoter; and h) a nucleic acid encoding at least one of the group consisting of cKit or a mutant thereof, fumarylacetoacetate hydrolase (Fah) or a mutant thereof, and any combination thereof. In some embodiments, the animal expresses at least one polypeptide selected from human M-CSF, human IL-3, human GM-CSF, human SIRPA, and human TPO. In one embodiment, the animal does not express Fah (Fah^{-/-}). In some embodiments, the animal comprises cKitw41 mutation or cKitWV mutation.

In one aspect, the present invention provides a genetically modified Rag-2^{-/-}, gamma chain^{-/-}, Fah^{-/-}, cKit^{w41/w41} non-human animal having a genome comprising a nucleic acid encoding at least one of the group consisting of human M-CSF, human IL-3, human GM-CSF, human SIRPA, and human TPO, operably linked to a promoter, wherein the mouse expresses at least one polypeptide selected from the group consisting of human M-CSF, human IL-3, human GM-CSF, human SIRPA, and human TPO.

In some embodiments, the animal comprises a genome comprising a nucleic acid encoding human M-CSF, a nucleic acid encoding human IL-3, a nucleic acid encoding human GM-CSF, a nucleic acid encoding human SIRPA and a nucleic acid encoding human TPO, wherein each of the nucleic acids encoding human M-CSF, human IL-3, human GN/I-CSF, human SIRPA, and human TPO is operably linked to a promoter, and wherein the animal expresses human M-CSF polypeptide, human IL-3 polypeptide, human GM-CSF polypeptide, human SIRPA polypeptide, and human TPO polypeptide.

In one embodiment, the animal is immunodeficient.

In one embodiment, the animal further comprises a genome comprising a nucleic acid encoding human IL-6. In one embodiment, the animal expresses human IL-6.

In one embodiment, the animal does not express recombination activating gene 2 (Rag-2^{-/-}). In one embodiment, the animal does not express IL2 receptor gamma chain (gamma chain^{-/-}). in another embodiment, the animal does not express Rag-2 and wherein the animal does not express IL2 receptor gamma chain (Rag-2^{-/-} gamma chain^{-/-}). In some embodiments, the animal does not express Rag-2, does not express IL2 receptor gamma chain, does not express Fah, and comprises cKitw41 mutation (Rag-2^{-/-} gamma chain^{-/-} Fah^{-/-} cKit^{w41/w41}) or cKitWV mutation (Rag-2^{-/-} gamma chain^{-/-} Fah^{-/-} cKitWV).

In some embodiments, the animal does not express SRB1 (SRB1^{-/-}), SRB2 (SRB2^{-/-}), or a combination thereof (SRB1^{-/-}SRB2^{-/-}).

In one embodiment, the animal is a rodent. In one embodiment, the animal is a mouse.

In one embodiment, the animal further comprises human hematopoietic cells. In another embodiment, the animal further comprises a human cancer cell. In one embodiment, the human cancer cell is a leukemia cell or a melanoma cell. In one embodiment, the animal further comprises a human liver cell. In another embodiment, the animal further comprises a human spleen cell.

In one embodiment, the animal has a sickle cell disease. In one embodiment, the animal is infected with malaria or hepatitis. In one embodiment, the animal has a liver disease. In various embodiments, the liver disease is human inflammatory disease, fatty liver disease, non-alcoholic steatohepatitis, or any combination thereof, in one aspect, the present invention provides a method of hematopoietic stem and progenitor cell (HSPC) engraftment in at least one genetically modified non-human animal described herein. In some embodiment, the method comprises the step of: administering at least one HSPCs to the genetically modified animal expressing at least one of the group consisting of human M-CSF, human IL-3, human GM-CST, human SIRPA, and human TPO; wherein the animal comprises a nucleic acid encoding at least one of the group consisting of cKit or a mutant thereof, fumarylacetoacetate hydrolase (Fah) or a mutant thereof, and any combination thereof; expresses at least one of the group consisting of human M-CSF, human IL-3, human GM-CSF, human SIRPA, and human TPO, does not express Fah (Fah^{-/-}); and comprises cKitw41 mutation or cKitWV mutation.

In another aspect, the present invention provides a method of human erythropoiesis in at least one genetically modified non-human animal described herein. In some embodiments, the method comprises the step of: administering at least one HSPCs to the genetically modified animal expressing at least one of the group consisting of human M-CSF, human IL-3, human GM-CSF, human SIRPA, and human TPO; wherein the animal comprises a nucleic acid encoding at least one of the group consisting of cKit or a mutant thereof, fumarylacetoacetate hydrolase (Fah) or a mutant thereof, and any combination thereof; expresses at least one of the group consisting of human M-CSF, human IL-3, human GM-CSF, human SIRPA, and human TPO, does not express Fah (Fah^{-/-}); and comprises cKitw41 mutation or eKitWV mutation.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary. It should be understood, however, that the invention is not limited to the precise arrangements and instrumentalities of the embodiments shown in the drawings.

FIG. 1 depicts the development of generation of MISTERG-FAH^{-/-} mouse.

FIG. 2 depicts human liver and RBC engraftment iii MISTERG-FAH mouse model. Six week old MISTERG-FAH mice were irradiated (150 Rads) and engrafted with I million primary human hepatocytes and 50,000 fetal liver CD34+ cells. Serum human albumin levels were measured by ELISA at indicated time points. Peripheral human RBCs (CD235a+) were measured by FACS at 11 weeks post co-engraftment.

FIG. 3 depicts a list of MISTERG-FAH^{-/-} cohorts engrafted with human hepatocytes and human CD34+ cells. * Tentative date

FIG. 4, comprising FIG. 4A through FIG. 4C, depicts irradiation or non-irradiation strategies to generate engrafted MISTERG-FAH^{-/-} mouse model. FIG. 4A depicts strategy 1:6-8 week old MISTERG-FAH mice were irradiated (150 Rads) and engrafted with 1-2 million primary human hepatocytes and 50,000 fetal liver or cord blood CD34⁺ cells. Strategy 2:6-8 week old MISTERG-FAH mice engrafted with 1-2 million primary human hepatocytes. One week after liver engraftment, mice were engrafted with 50,000 fetal liver or cord blood CD34⁻ cells without irradiation. FIG. 4B depicts serum human albumin levels and frequency of peripheral human RBCs were measured by ELISA or FACS between 10-14 weeks post engraftment. FIG. 4C depicts survival curve of engrafted MISTERG-FAH mice with indicated treatment. Liver: 1-2 million human hepatocytes; IR: irradiation (150 rads); CD34: 50,000 fetal liver or cord blood CD34+ cells.

FIG. 5 depicts the generation of MISTRG mice and the improvement of humanized mice by human cytokine knock-in gene replacement. MISTRG mice were generated via knock-in technology to express non-crossreactive, human cytokines from the corresponding murine loci in the huSIRPαRag^{-/-}γ^{-/-} background. Mutation of the murine c-kit gene to improve overall engraftment and specifically erythropoiesis, and homozygous deletion of the fumarylacetoacetate hydrolase (Fah) to humanize the liver in the context of MISTRG mice.

FIG. 6, comprising FIG. 6A through FIG. 6I, depicts the results of exemplary experiments showing that MISTRG mice have improved overall engraftment and BM erythropoiesis with erythroid dysplasia. NSG and MISTRG mice were engrafted with 10⁵ healthy BM derived CD34+ cells and analyzed >12 weeks after engraftment. MISTRG efficiently engrafted adult 13141 CD34+ cells with BM erythropoiesis but absent RBC in PB. FIG. 6A depicts an overall engraftment (huCD45⁺) in BM. FIG. 6B depicts an overall engraftment (huCD45⁺) in PB. FIG. 6C depicts percentage of erythroid lineage (htiCD45⁻ muCD45⁻ huCD71⁺ and/or CD235(GPA)⁺) in whole BM (WBM). FIG. 6D depicts bone marrow of MISTRG and NSG. FIG. 6E depicts an overall engraftment (huCD45⁺) in BM. FIG. 6F depicts the results of exemplary experiments showing an overall higher engraftment in MISTRG mice with engraftment levels >10% than NSG. FIG. 6G depicts percentage of erythroid lineage (huCD45− muCD45− huCD71+ and/or CD235(GPA)+) in whole BM (WBM) obtained from MDS (myelodysplastic syndrome) samples. FIG. 6H depicts representative example of Ring Sideroblasts in MISTRG and patient BM but not NSG BM engrafted with SF3B1 mutant low-grade MDS. FIG. 6I depicts the BM histology.

FIG. 7, comprising FIG. 7A and FIG. 7B, depicts results that demonstrate that c-kit mutant MISTRG allow engraftment without irradiation and support robust erythropoiesis from healthy adult BM derived CD34+ cells. Overall (huCD45⁺) engraftment in PB and BM in non-irradiated MISTRG 16 weeks of transplant and heterozygous c-kit mutant MISTRG (MISTITRG k^{W/+}). FIG. 7A depicts percentage of glycophorin A (GPA, huCD235) positive erythropoiesis bone marrow biopsies from MISTRG k^{W/+} and NtISTRG mice. FIG. 7B depicts the presence of glycophorin A (GPA, huCD235) positive erythropoiesis in bone marrow biopsies from MISTRG k^{W/+} mice. RBC are absent in peripheral blood. HuRBC are rapidly cleared from the circulation and are trapped in the mouse liver, HuRBC (CFSE) and muRBC (violet) were injected into MISTRG mice and tracked in PB over time.

FIG. 8, comprising FIG. 8A through FIG. 8C, depicts results that demonstrate that MISTRG mice develop “double-human” erythroblastic islands. FIG. 8A depicts representative images of lineage positive cells in BM of engrafted NSG vs MISTRG mice including myeloid (hCD15, hCD68) and erythroid (hCD235) lineage cells and central CD169+MΦs in MISTRG but not NSG BM. FIG. 8B depicts central CD169+MΦs in MISTRG BM. FIG. 8C depicts central CD169+MΦs in MISTRG BM within erythroblastic islands.

FIG. 9, comprising FIG. 9A through FIG. 9D, depicts results that demonstrate that liposomal clodronate (LC) transiently spares PB RBC in MISTRG mice with low efficiency with abrogation of human CD169 central MΦs, and with significant toxicity. LC was injected for 5 days into engrafted MISTRG and huRBC in PB measured. via flow cytometry. Liposomal clodronate rescued RBC survival but abrogated central EBI macrophages and was toxic. FIG. 9A depicts results that demonstrate that HuRBC increase to ˜1% of all RBC. FIG. 9B depicts results that demonstrate that HuRBC increase to ˜1% of all RBC with improved maturation as evident by reduced CD71 expression. FIG. 9C depicts results that demonstrate LC abrogates BM human central MΦs. FIG. 9D depicts results that demonstrate LC carries significant lethality.

FIG. 10, comprising FIG. 10A through FIG. 10C, depicts results that demonstrate that HuRBC are rapidly cleared from the circulation and are trapped in the mouse liver. HuRBC (CFSE) and muRBC (violet) were injected into MISTRG mice and tracked in PB over time, Human RBC did not survive in mice and got sequestered in the murine host's liver vasculature. FIG. 10A depicts results that demonstrate that HuRBC are rapidly cleared from circulation. FIG. 10B depicts in vivo imaging of MISTRG liver after injection of CFSE labeled huRBC. FIG. 10C depicts in vivo imaging of MISTRG liver after injection of CFSE labeled muRBC. HuRBC are trapped in the mouse liver and show markedly reduced flow while mouse RBC quickly passage through the liver vessels (marked with red fluorescent dextran particles).

FIG. 11, comprising FIG. 11A through FIG. 11L, depicts results that demonstrate that HuHep.MISTRGFah efficiently engraft with robust EP and mature huRBC in circulation. FIG. 11A depicts results that demonstrate HuAlbumin, huM45+, CD3, CD19, and CD33 in MIS RG and HuHep MISTRGFah mice. FIG. 11B depicts results that demonstrate huCD45+ engraftment in MISTRG, HuHep MISTRGFah, NSG W41, and MISTRG W41 mice. FIG. 11C depicts results that demonstrate huCD235+ EP in BM in MISTRG, HuHep MISTRGFah, NSG W41, and MISTRG W41 mice. FIG. 11D depicts huEP in spleen in engrafted HuHep-MISTRGFah mice. FIG. 11E depicts results that demonstrate that only HuHepMISTRGFah and to a lesser degree MISTRGW41 mice have circulating PB huRBC. Only HuHepMISTRGFah and to a lesser degree MISTRGW41 mice had circulating PB huRBC. Only HuHepMISTRGFah lack muC3 coating of EP in BM. FIG. 11F depicts the results that demonstrate that only HutiepMISTRGFah and to a lesser degree MISTRGW41 mice have circulating PB huRBC (red circles) with lack of muC3 coating of EP in BM (pink circle). Only HuHepMISTRGFah lack muC3 coating of EP in BM. FIG. 11G depicts the results that demonstrate HuHepMISTRGFah with lack of muC3 coating of EP in BM. Only HuHepMISTRGFah and to a lesser degree MISTRGW41 mice have circulating PB huRBC. FIG. 11H depicts results that demonstrate that HuHepMISTRGFah livers show full replacement of murine with human liver MΦs. FIG. 11I depicts Hu CD45+ in PB in MISTRG and HiHepMISTRG FAH. HuHep MISTRG achieve full liver humanization, efficiently engraft huCD34+ and give rise to PB leukocytes. FIG. 11J depicts the exemplary results showing higher huCD235a+ in HuHepMISTRG Fah when compared to MISTRG in BM FIG. 11K depicts the exemplary results showing higher huCD235a+ in HuHepMISTRG Fah when compared to MISTRG in PB. FIG. 11L depicts exemplary results that demonstrate high bone marrow engraftment in HuHepMISTRGFah when compared to MISTRG.

FIG. 12, comprising FIG. 12A and FIG. 12F, depicts results that demonstrate Sickle cell engraftment (prelim at 6 weeks) in MISTRGW41 mice. FIG. 12A depicts PB (top) and BM of one select mouse (red dot) (bottom) engrafted with 10⁵ CD34 cells from SCD BM. FIG. 12B depicts depletion of murine tissue MΦs with anti-mouse Ccr2 antibody partially rescues PB huRBC. HuCD34+ engrafted MISTRG mice were injected with 1 mg/kg anti-Ccr2 antibody (or isotype control) every other day for 5 days and presence of huRBC in PB was determined via flow-cytometry (huCD71, huCD235 staining within human cells) before and after treatment. FIG. 12C depicts exemplary results that demonstrate erythroid enucleation and maturation in BM (gated on huCD45-muCD45-cells). FIG. 12D depicts exemplary results that demonstrate erythroid enucleation and maturation in PB (gated on huCD45-muCD45-cells). FIG. 12E depicts exemplary results demonstrating that complement C3 coats erythroid precursors in MISTRG but not in HuHep MISTRGFah mice. FIG. 12F depicts huCD235a+ in BM in MISTRG and HuHepMISTRGFah.

FIG. 13 depicts the limited biological cross-reactivity of signals critical for hematopoiesis.

FIG. 14 depicts the improvement of humanized mice by cytokine knock-in gene replacement.

FIG. 15 depicts the process of improving erythropoiesis by boosting human hematopoiesis.

FIG. 16, comprising FIG. 16A and FIG. 16B, depicts results that, demonstrate that increased frequency of human erythroid cells in the bone marrow but not peripheral blood of hEPO KI mice, FIG. 16A depicts the results obtained from human CD235+ erythrocyte engraftment that was monitored after 6-8 weeks in Rag2^{-/-}I12rg^{-/-} Tpoh/hGmcsf/I13h1hMesth/h with the indicated combinations of EPO and hSIRPa. FIG. 16B depicts summary of erythroid cell engraftment in the Bone marrow.

FIG. 17 depicts results that demonstrate that human macrophages are important to the development of human erythrocytes. Human CD235+ erythrocytes in the peripheral blood were monitored after 6-8 weeks in mice with the indicated mouse strains.

FIG. 18 depicts results that demonstrate that low frequency of human erythrocytes in blood of MITERGSKI or TIERGSKI mice.

FIG. 19 depicts that phagocytosis of erythrocytes and other cells is primarily regulated via two processes.

FIG. 20 depicts transfused human RBCs and de novo produced human RBCs.

FIG. 21 depicts alternative complement activation pathway.

FIG. 22 depicts results that demonstrate that complement knockout is not sufficient for the reconstitution of human erythrocytes in the peripheral blood. Human CD235+ erythrocytes in the peripheral blood were monitored after 6-8 weeks in mice with the indicated mouse strains.

FIG. 23 depicts results that demonstrate improved level of peripheral human erythrocytes after Phagocyte depletion by clodronate in engrafted TIERGSKI mice. Clodronate treatment increases circulating human erythroid cells/reticulocytes in HSC-engrafted TIERGSKI mice, 7 weeks after HSC-engraftment, TIERGSKI mice were treated with daily retro-orbital injection of 50 μl clodronate liposome for five consecutive days. Frequency of human CD235+ or CD235+ /CD71+ cells (human reticulocytes) in the peripheral blood before or after treatment was measure by FACS.

FIG. 24 depicts P. falciparum infection of mice with improved erythropoiesis.

FIG. 25 depicts ex vivo P. falciparum infection of human erythrocytes from engrafted TIERGSKI mice. Infection of humanized mouse blood with P. falciparum blood stage parasites: Purified human red blood cells containing schizonts (99% purity) were added to humanized mouse and control mouse blood.

FIG. 26 depicts results that demonstrate that P. falciparum blood stage parasites infected erythrocytes from engrafted TIERGSKI mice. Infection of humanized mouse blood with P. falciparum blood stage parasites: Purified human red blood cells containing schizonts (99% purity) were added to humanized mouse and control mouse blood. Samples were collected 48 or 90 hours after infection. Hoechst, anti-human Band3 and anti-mouse Ter119 antibodies were used for staining. Green: mouse Ter119; Red: anti-human Band3; Blue: Hoechst.

FIG. 27 depicts invasion of blood stage P. falciparum parasites in human erythrocytes from engrafted TIERGSKI mice. Infection of humanized mouse blood with P. falciparum blood stage parasites: Purified human red blood cells containing schizonts (98% purity) were added to humanized mouse and control mouse blood at a 1% het, Parasites invasion was checked 20 hours later. Parasitized erythrocyte multiplication rate (PEMR) was calculated from the final ring number over the initial schizont number. #1 mouse has 3.5% hRBC in the periphery. The rest was around 1%.

FIG. 28 depicts multiplication of blood stage P. falciparum parasites in human erythrocytes from engrafted TIERGSKI mice. Infection of humanized mouse blood with P. falciparum blood stage parasites: Purified human red blood cells containing schizonts (98% purity) were added to humanized mouse and control mouse blood at a 1% hct. Parasites multiplication was checked 60 hour later. Parasitized erythrocyte multiplication rate (PMR) was calculated from the rings (20 hours) to rings (60 hours).

FIG. 29 depicts ex vivo P. falciparum infection of human erythrocytes from engrafted TIERGSKI mice. Infection of humanized mouse blood with P. falciparum blood stage parasites: Purified human red blood cells containing schizonts (99% purity) were added to humanized mouse and control mouse blood. Fresh human RBCs were added into the culture 48 hours post infection and infection culture was maintained for additional 10 days.

FIG. 30 depicts multiplication of blood stage P. falciparum parasites in human erythrocytes from engrafted TIERGSKI mice. The results demonstrated that falciparum blood stage parasites complete life cycles and proliferates in human RBCs produced from HSC-engrafted TIERGSKI mice. Purified human red blood cells containing schizonts (99% purity) were added to humanized mouse and control mouse blood. Fresh human RBCs were added into the culture 48 hours post infection and infection culture was maintained for additional 10 days. Giemsa staining and SYBR green staining were performed to quantify parasitemia.

FIG. 31 depicts multiplication of blood stage P. falciparum parasites in human erythrocytes from engrafted TIERGSKI mice. These results demonstrated that falciparum blood stage parasites complete life cycles and proliferates in human RBCs produced from HSC-engrafted TIERGSKI mice, Purified human red blood cells containing schizonts (99% purity) were added to humanized mouse and control mouse blood. Fresh human RBCs were added into the culture 48 hours post infection and infection culture was maintained for additional 10 days. Giemsa staining and SYBR green staining were performed to quantify parasitemia.

FIG. 32 depicts P. falciparum infection of engrafted TIERGSKI mice with improved erythropoiesis. Infection of humanized mice with P. falciparum blood stage parasites: TIERGSKI mice were treated with clodronate for four consecutive days. Purified human red blood cells containing schizont stage P. falciparum were transfused into engrafted mice or control mice million infected cells per mouse), Peripheral blood was collected from each mouse and stained with anti-hCD235a, anti-hCM I and anti-mTer119 antibodies, as well as Hoechst. hCD235a-negative cells (mouse red blood cells) were excluded from further analysis.

FIG. 33 depicts the results of in vivo infection of humanized mice with P. falciparum blood stage parasites. Infection of humanized mice with P. falciparum blood stage parasites: TIEROSKI mice were treated with clodronate for four consecutive days. Purified human red blood cells containing schizont stage P. falciparum were transfused into engrafted mice or control mice (5 million infected cells per mouse). Peripheral blood was collected from each mouse for Giemsa staining.

FIG. 34 depicts in vivo infection of humanized mice with P. falciparum blood stage parasites. Infection of humanized mice with P. falciparum blood stage parasites: TIERGSKI mice were treated with clodronate for four consecutive days. Purified human red blood cells containing schizont stage P. falciparum were transfused into engrafted mice or control mice (5 million infected cells per mouse). Peripheral blood was collected from each mouse and stained with anti-hCD235a, anti-hCD71 and anti-mTer119 antibodies, as well as Hoechst. hCD235a-negative cells (mouse red blood cells) were excluded from further analysis. Infected human red blood cells by Hoechst staining 48 hours post infection were shown. % parasitemia at day 2 and day 5 post infection was also shown.

FIG. 35 depicts infection of humanized mice with P. falciparum blood stage parasites. Infection of humanized mice with P. falciparum blood stage parasites: TIERGSKI mice were treated with clodronate for four consecutive days. Purified human red blood cells containing schizont stage P. falciparum were transfused into engrafted mice or control mice (5 million infected cells per mouse). Peripheral blood was collected from each mouse and stained with anti-hCD235a, anti-hCD71 and anti-mTer119 antibodies, as well as Hoechst. hCD235a-negative cells (mouse red blood cells) were excluded from further analysis. Infected human red blood cells by Hoechst staining 48 hours post infection were shown. % parasitemia at day 2 and day 5 post infection was also shown.

FIG. 36 depicts ex vivo P. vivax infection of human erythrocytes from engrafted TIERGSKI mice

FIG. 37 depicts the experimental layout for infecting and treating group A (engrafted animal), group B (non engrafted animals) by clodronate.

FIG. 38 depicts results that demonstrate human RRCs in circulation.

FIG. 39 depicts parasitemia FACS data for mouse #6 and uninfected mouse #10 after 48 hr pre-infection, 24 hr post-infection, and 72 hr post-infection.

FIG. 40 depicts an exemplary ring in human RBC and exemplary ring in mouse RBC.

FIG. 41 depicts clearance of human red blood cells in the mouse peripheral blood. Human and mouse red blood cells were pre-labeled with CFSE or violet dye, respectively. After blood infusion (retro-orbital), peripheral blood or various tissues from RGSKI mice were collected at indicated time points.

FIG. 42 depicts rapid clearance of human red blood cells in the mouse peripheral blood. Human and mouse red blood cells were pre-labeled with CFSE or violet dye, respectively. Mixed blood sample and blood collected 5 minutes after (retro-orbital) were analyzed by FACS.

FIG. 43 depicts rapid clearance of human red blood cells in the mouse peripheral blood as demonstrated via the remaining human RBCs/remaining mouse RBC. Human and mouse red blood cells were pre-labeled with CFSE or violet dye, respectively. After blood infusion (retro-orbital), peripheral blood or various tissues from RGSKI mice were collected at indicated time points and were analyzed by FACS.

FIG. 44 depicts clearance of human red blood cells in the mouse peripheral blood. Human and mouse red blood cells were pre-labeled with CFSE or violet dye, respectively. After blood infusion (retro-orbital), peripheral blood or various tissues from RGSKI mice were collected at indicated time points and were analyzed by PACS.

FIG. 45 depicts rapid accumulation of infused human red blood cells in mouse tissues. Human and mouse red blood cells were pre-labeled with CFSE or violet dye, respectively. After blood infusion (retro-orbital), peripheral blood or various tissues from RGSKI mice were collected at indicated time points and were analyzed by FACS.

FIG. 46 depicts rapid accumulation of infused human red blood cells in mouse liver as demonstrated via the percent injected RBCs in total RBCs. Human and mouse red blood cells were pre-labeled with CFSE or violet dye, respectively. After blood infusion (retro-orbital), peripheral blood or various tissues from RGSKI mice were collected at indicated time points and were analyzed by FAGS.

FIG. 47 depicts rapid accumulation of infused human red blood cells in mouse spleen as demonstrated via the percent injected RBCs in total RBCs. Human and mouse red blood cells were pre-labeled with CFSE, or violet dye, respectively, After blood infusion (retro-orbital), peripheral blood or various tissues from RGSKI mice were collected at indicated time points and were analyzed by FACS.

FIG. 48 depicts results that demonstrate that no accumulation of infused human red blood cells occurred in mouse lung. Human and mouse red blood cells were pre-labeled with CFSE or violet dye, respectively. After blood infusion (retro-orbital), peripheral blood or various tissues from RGSKI mice were collected at indicated time points and were analyzed by FAGS.

FIG. 49 depicts results that demonstrate that no accumulation of infused human red blood cells occurred in mouse bone marrow. Human and mouse red blood cells were pre-labeled with CFSE or violet dye, respectively. After blood infusion (retro-orbital), peripheral blood or various tissues from RGSKI mice were collected at indicated time points and were analyzed by FACS.

FIG. 50 depicts rapid accumulation of infused human red blood cells in mouse liver by live animal imaging. After anesthesia procedure, mouse was given rhodamine labeled dextran by I.V. 5 mins later, mouse and human RBCs were labeled with CFSE and infused into mouse through i.v. separately.

FIG. 51 depicts druglantibody screening to identify mouse liver receptor(s) that mediates human RBC sequestration. RGSKI mice were treated with different antibodies or inhibitors to block various macrophage receptors. One hour after treatment, mice were infused with human RRCs and peripheral blood was collected for flow analysis.

FIG. 52 depicts results that demonstrate that transfused human red blood cells in mouse peripheral blood are protected by D-4F. Apolipoprotein A1 mimetic peptide D-4F blocks in vivo destruction of human RBCs. RGSKI mice were treated with different antibodies or inhibitors to block various macrophage receptors. One hour after treatment, mice were infused with human RBCs and peripheral blood was collected at indicated time points. Anti-Dectin: 50 ug/inouse; Anti-CD169:50 ug/mouse; Anti-CD169 clone 3D6:100 ug/mice; Anti-Mannose receptor: 50 ug/mouse; D-4F: 10 mg/mouse; Asialofetuin: 10 mg/mouse; Control: PBS,

FIG. 53 depicts results that demonstrate that transfused human red blood cells in mouse peripheral blood are protected by D-4F.

FIG. 54 depicts scavenger receptors and their ligands.

FIG. 55 depicts results that demonstrate that scavenger receptor class A and CD36 (one member of class B) inhibitors do not block destruction of transfused human RBCs in mouse system. Inhibitor of scavenger receptor B1 can protect human RBCs in the mouse system. RGSKI mice were treated with class A inhibitors or anti-CD36. One hour alter treatment, mice were infused with human RBCs and peripheral blood was collected at indicated time points.

FIG. 56 depicts results that demonstrate that transfused human red blood cells in mouse periphery are protected by scavenger receptor B1 (SR-B1) inhibitor BLT-1. Inhibitor of scavenger receptor B1 can protect human RBCs in the mouse system. RGSKI mice were treated with scavenger receptor B1 named BLT-1 (2 mg per mouse). One hour after treatment, mice were infused with human RBCs and peripheral blood was collected at indicated time points. Control: PBS.

FIG. 57 depicts results that demonstrate that transfused human red blood cells in mouse peripheral blood are protected by a second SR-B1 inhibitor ITX7650. Inhibitor of scavenger receptor B1 can protect human RBCs in the mouse system. RGSKI mice were treated with scavenger receptor B1 named ITX7650 or ITX5061 (1 mg per mouse) obtained from iTherX. One hour after treatment, mice were infused with human RBCs and peripheral blood was collected at indicated time points. Control: PBS.

FIG. 58 depicts experimental timeline.

FIG. 59 depicts clodronate limits phagocytes of hRBC. The results demonstrated that circulating hRBCs depends on the success of CD34+ engraftment.

FIG. 60 depicts circulating human red blood cells in MISTEKI mice. The data indicated that clodronate progressively limited phagocytosis.

FIG. 61 depicts results of circulating human red blood cells in NUSTEK1 mice that demonstrate that most hRBCs in circulation are reticulocytes as well as the survival of normocytes,

FIG. 62 depicts the impact of clodronate and infection on the spleen. Spleen size reduced in clodronate-treated animals (when not infected; left) and became darker/bigger in infected animals (animal 6, 8) where parasitemia was detected (right).

FIG. 63 depicts a schematic representation of MISTRW41Fah^{-/-}model engrafted with human RBC, human Sickle Cells, and mouse liver cells.

FIG. 64 depicts differences in the pathogenic mechanisms between human and mouse NAFLD.

FIG. 65 depicts the exemplary results that demonstrate that MISTRG-Fah support the growth of human hematopoiesis and human hepatocytes. NTBC is an inhibitor of toxic tyrosine metabolite production that rescues the Fah-KO mouse hepatocytes from death,

FIG. 66 depicts description of MISTRG-6 mice.

FIG. 67 depicts the exemplary results that demonstrate that MISTRG-6 mice support the growth of human Kupffer cells, human endothelial cells and human stellate cells as it was shown by flow cytometry and Immunohistochemistry.

FIG. 68 depicts a schematic representation of workflow for the establishment of a “human” liver. The humanization is evaluated with FACS, Immunohistochemistry (IHC) and Single-cell RNA sequencing. CV: Central Vein, PV: Portal Vein, HA: Hepatic Artery.

FIG. 69 depicts a schematic representation of workflow for the establishment of NAFLD animal model in a “human” liver. The development of human features of NASH is expected.

FIG. 70 depicts a schematic representation of a study where a mouse with human liver is a unique translation tool to examine whether the findings in the popular mouse models are translatable to human cell in vivo and whether the dosage or toxicity of a drug is similar between rodent and human cells in vivo,

FIG. 71 depicts a schematic representation of workflow for single cell RNA-seq analysis, A representative image from gut single cell RNA-seq. Each color is a different cell type.

### DETAILED DESCRIPTION

The invention relates generally to genetically modified non-human animals comprising a nucleic acid encoding at least one of cKit or a mutant thereof and fumarylacetoacetate hydrolase (Fah) or a mutant thereof; and expressing at least one of human M-CSF, human IL-3, human GM-CSF, human SIRPA or human TPO. The invention also relates to methods of generating and methods of using the genetically modified non-human animals described herein. In some embodiments, the genetically modified non-human animal is a mouse. In some embodiments, the genetically modified non-human animal described herein is engrafted with human hematopoietic cells. In various embodiments, the human hematopoietic cell-engrafted, genetically modified non-human animals of the invention are useful for the in vivo evaluation of the growth and differentiation of hematopoietic and immune cells, for the in vivo evaluation of human hematopoiesis, for the in vivo assessment of human erythropoiesis, for the in vivo evaluation of cancer cells, for the in vivo evaluation of diseases associated with deficiencies in the production of red blood cells, for the in vivo evaluation of a treatment of diseases associated with deficiencies in the production of red blood cells, for the in vivo evaluation of diseases associated with genetic deficiencies in the production of red blood cells, for the in vivo evaluation of a treatment of diseases associated with genetic deficiencies in the production of red blood cells, for the in vivo evaluation of human inflammatory diseases in the liver, for the in vivo evaluation of a treatment of human inflammatory disease in the liver, for the in vivo evaluation of fatty liver disease, for the in vivo evaluation of a treatment of fatty liver disease, for the in vivo evaluation of non-alcoholic steatohepatitis, for the in vivo evaluation of a treatment of non-alcoholic steatohepatitis, for the in vivo evaluation of hepatitis, for the in vivo evaluation of a treatment of hepatitis, for the in vivo evaluation of anemia, for the in vivo evaluation of sickle cell disease, for the in vivo evaluation of malaria, for the in vivo evaluation of infection, for the in vivo evaluation of liver diseases, for the in vivo evaluation of a treatment of anemia, for the in vivo evaluation of a treatment of sickle cell disease, for the in vivo evaluation of a treatment of malaria, for the in vivo evaluation of a treatment of infection, for the in vivo evaluation of a treatment of liver diseases, for the in vivo assessment of an immune response, for the in vivo evaluation of vaccines and vaccination regimens, for the use in testing the effect of agents that modulate cancer cell growth or survival, for the in vivo evaluation of a treatment of cancer, for the in vivo production and collection of immune mediators, including human antibodies, and for use in testing the effect of agents that modulate hematopoietic and immune cell function.

Definitions

Unless defined otherwise, all technical and scientific terms used herein have the same meaning as commonly understood by one of ordinary skill in the art to which this invention belongs. Such terms are found defined and used in context in various standard references illustratively including J. Sambrook and D. W. Russell, Molecular Cloning: A Laboratory Manual, Cold Spring Harbor Laboratory Press; 3rd Ed., 2012; F. M. Ausubel, Ed., Short Protocols in Molecular Biology, Current Protocols; 5th Ed., 2002; B. Alberts et al., Molecular Biology of the Cell, 4th Ed., Garland, 2002; L. Nelson and M. M. Cox, Lehninger Principles of Biochemistry, 4th Ed., W. H. Freeman & Company, 2004; and Herdewijn, P. (Ed.), Oligonucleotide Synthesis: Methods and Applications, Methods in Molecular Biology, Humana Press, 2004. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antibody,” as used herein, refers to an immtmoglobulin molecule which is able to specifically bind to a specific epitope on an antigen. Antibodies can be intact immunoglobulins derived from natural sources or from recombinant sources and can be immunoreactive portions of intact immunoglobulins The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; 1arlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “cancer” as used herein is defined as disease characterized by the uncontrolled proliferation and/or growth of aberrant cells. Cancer cells can spread locally or through the bloodstream and lymphatic system to other parts of the body. Examples of various cancers amenable to the invention include, but are not limited to, breast cancer, prostate cancer, ovarian cancer, cervical cancer, skin cancer, pancreatic cancer, colorectal cancer, renal cancer, liver cancer, bone cancer, brain cancer, lymphoma, leukemia, lung cancer and the like.

“Constitutive” expression is a state in which a gene product is produced in a living cell under most or all physiological conditions of the cell.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for ammo acid. residues which are not present in the mature protein encoded by the mRNA molecule (e,g., ammo acid residues in a protein export signal sequence).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of at least one sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can he referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The terms “expression construct” and “expression cassette” are used herein to refer to a double-stranded recombinant DNA molecule containing a desired nucleic acid human coding sequence and containing one or more regulatory elements necessary or desirable for the expression of the operably linked coding sequence.

As used herein, the term “fragment,” as applied to a nucleic acid or polypeptide, refers to a subsequence of a larger nucleic acid or polypeptide. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between). A “fragment” of a polypeptide can be at least about 15 nucleotides in length; for example, at least about 50 amino acids to about 100 amino acids; at least about 100 to about 500 amino acids, at least about 500 to about 1000 amino acids, at least about 1000 amino acids to about 1500 amino acids; or about 1500 amino acids to about 2500 amino acids; or about 2500 amino acids (and any integer value in between).

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

“Homologous” as used herein, refers to the subunit sequence similarity between two polymeric molecules, e.g. between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e,g. if half (e.g,, five positions in a polymer ten subunits in length) of the positions in two compound sequences are homologous then the two sequences are 50% homologous, if 90% of the positions, e.g. 9 of 10, are matched or homologous, the two sequences share 90% homology, By way of example, the DNA sequences 5′-ATTGCC-3′ and 5′-TATGGC-3′ share 50% homology.

The terms “human hematopoietic stem and progenitor cells” and “human HSPC” as used herein, refer to human self-renewing multipotent hematopoietic stern cells and hematopoietic progenitor cells.

“inducible:” expression is a state in which a gene product is produced in a living cell in response to the presence of a signal in the cell.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “operably linked” as used herein refers to a polynucleotide in functional relationship with a second polynucleotide. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region. In some embodiments, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds, A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The term “peptide” typically refers to short polypeptides. The term “protein” typically refers to large polypeptides.

The term “progeny” as used herein refers to a descendent or offspring and includes the differentiated or undifferentiated decedent cell derived from a parent cell. In one usage, the term progeny refers to a descendent. cell which is genetically identical to the parent. In another use, the term progeny refers to a descendent cell which is genetically and phenotypically identical to the parent. In yet another usage, the term progeny refers to a descendent cell that has differentiated from the parent cell.

The term “promoter” as used herein refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

An included promoter can be a constitutive promoter or can provide inducible expression; and can provide ubiquitous, tissue-specific or cell-type specific expression.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A “recombinant polypeptide” is one, which is produced upon expression of a recombinant polynucleotide.

The term “regulatory element” as used herein refers to a nucleotide sequence which controls some aspect of the expression of nucleic acid sequences. Exemplary regulatory elements illustratively include an enhancer, an internal ribosome entry site (IBES), an intron; an origin of replication, a polyadenylation signal (pA), a promoter, an enhancer, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation. Expression constructs can be generated recombinantly or synthetically using well-known methodology.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the term “genetically modified” means an animal, the germ cells of which comprise an exogenous human nucleic acid or human nucleic acid sequence, By way of non-limiting examples a genetically modified animal can be a transgenic animal or a knock-in animal, so long as the animal comprises a human nucleic acid sequence.

As used herein, “knock-in” is meant a genetic modification that replaces the genetic information encoded at a chromosomal locus in a non-human animal with a different DNA sequence.

Description

The invention relates to a genetically modified non-human animal expressing human M-CSF, human IL-3/GM-CSF, human SIRPA, and human TPO (herein referred to as MIST). The invention also relates to methods of generating and methods of using the genetically modified non-human animals described herein. In some embodiments, the genetically modified non-human animal is a mouse. In some embodiments, the genetically modified non-human animal is an immunodeficient mouse. In a particular embodiment, the immunodeficient mouse is a RAG2^{-/-}γ_{c}^{-/-} mouse. In one embodiment, the genetically modified non-human animal of the invention expresses human M-CSF, human IL-3/GM-CS, human SIRPA, and human TPO, also expresses Fah (referred to herein as MIST-Fah^{+/+}). In one embodiment, the genetically modified non-human animal of the invention expresses human M-CSF, human IL-3/GM-CSF, human SIRPA, and human TPO, expresses mutant Fah (referred to herein as MIST-Fah^{+/-}). In one embodiment, the genetically modified non-human animal of the invention expresses human M-CSF, human IL-3/GM-CSF, human SIRPA, and human TPO, and does not express Fah (referred to herein as MIST-Fah^{-/-}).

In one embodiment, the genetically modified non-human animal of the invention expresses human M-CSF, human IL-3/GM-CSF, human SIRPA, and human TPO, expresses mutant c-kit w41 (referred to herein as NI ISTW41). In one embodiment, the genetically modified non-human animal of the invention expresses human M-CSF, human IL-3/GM-CSF, human SIRPA, and human TPO, expresses homozygous mutant c-kit w41/w41 (referred to herein as MIST^{w41/w41}). In one embodiment, the genetically modified non-human animal of the invention expresses human M-CSF, human IL-3/GM-CSF, human SIRPA F, and human TPO, expresses mutant c-kit w41, and does not express Fah (referred to herein as MISTW41-Fah^{-/-}). In one embodiment, the genetically modified non-human animal of the invention expresses human M-CSF, human IL-3/GM-CSF, human SIRPA, and human TPO, expresses homozygous mutant c-kit w41/w41, and does not express Fah (referred to herein as MIST^{w41/w41}Fah^{-/-}).

In one embodiment, the genetically modified non-human animal of the invention expresses human M-CSF, human IL-3/GM-CSF, human SIRPA, and human TPO, expresses heterozygous c-kit mutant (referred to herein as MIST^{w/+}). In one embodiment, the genetically modified non-human animal of the invention expresses human M-CSF, human IL-3/GM-CSF, human SIRPA, and human TPO, expresses mutant c-kit wv (referred to herein as MISTekitwv). In one embodiment, the genetically modified non-human animal of the invention expresses human M-CSF, human IL-3/GM-CSF, human SIRPA, and human TPO, expresses heterozygous c-kit mutant, and does not express Fah (referred to herein as MIST^{w41/+}-Fah^{631 /-}). In one embodiment, the genetically modified non-human animal of the invention expresses human M-CSF, human IL-3/GM-CSF, human SIRPA, and human TPO, expresses mutant c-kit wv, and does not express Fah (referred to herein as MISTckitwvFah^{-/-}).

In another particular embodiment, the genetically modified non-human animal of the invention expresses human M-CSF, human IL-3/GM-CSF, and human TPO and does not express RAG2 or γ_{c} (referred to herein as MITRG). In one embodiment, the genetically modified non-human animal of the invention expresses human M-CSF, human IL-3/GM-CSF, and human TPO, also expresses Fah, and does not express RAG2 or (referred to herein as MITRG-Fah^{+/+}). In one embodiment, the genetically modified non-human animal of the invention expresses human M-CSF, human IL-3/GM-CSF, and human TPO, expresses mutant Fah, and does not express RAG2 or γ_{c} (referred to herein as MITRG-Fah^{+/-}). In one embodiment, the genetically modified non-human animal of the invention expresses human M-CSF, human IL-3/GM-CSF, and human TPO, does not express Fah, and does not express RAG2 or γ_{c} (referred to herein as MITRG-Fah^{-/-}).

In one embodiment, the genetically modified non-human animal of the invention expresses human M-CSF, human IL-3/GM-CSF, and human TPO, expresses mutant c-kit w41, and does not express RAG2 or γ_{c} (referred to herein as MITRGW41). In one embodiment, the genetically modified non-human animal of the invention expresses human M-CSF, human IL-3/GM-CSF, and human TPO, expresses homozygous mutant c-kit w41/w41, and does not express RAG2 or γ_{c} (referred to herein as MITRG^{w41/w41}). In. one embodiment, the genetically modified non-human animal of the invention expresses human M-CSF, human IL-3/GM-CSF, and human TPO, expresses mutant c-kit w41, and does not express Fah, and does not express RAG2 or γ_{c} (referred to herein as MITRGW41-Fah^{-/-}). In one embodiment, the genetically modified non-human animal of the invention expresses human M-CSF, human IL-3/GM-CSF, and human TPO, expresses homozygous mutant c-kit w41/w41, and does not express Fah, and does not express RAG2 or γ_{c} (referred to herein as MITRG^{w41/w41}Fah^{-/-}).

In one embodiment, the genetically modified non-human animal of the invention expresses human M-CSF, human IL-3/GM-CSF, and human TPO, expresses heterozygous c-kit mutant, and does not express RAG2 or γ_{c} (referred to herein as MITRG^{w/+}). In one embodiment, the genetically modified non-human animal of the invention expresses human M-CSF, human IL-3/GM-CSF, and human TPO, expresses mutant c-kit wv, and does not express RAG2 or γ_{c} (referred to herein as MITRGckitwv). In one embodiment, the genetically modified non-human animal of the invention expresses human M-CSF, human IL-3/GM-CSF, and human TPO, expresses heterozygous c-kit mutant, and does not express Fah, and does not express RAG2 or γ_{c} (referred to herein as MITRG^{w/+}-Fah^{-/-}). In one embodiment, the genetically modified non-human animal of the invention expresses human M-CSF, human IL-3/GM-CSF, and human TPO, expresses mutant c-kit wv, and does not express Fah, and does not express RAG2 or γ_{c} (referred to herein as MITRGekitwvFah^{-/-}).

In another particular embodiment, the genetically modified non-human animal of the invention expresses human M-CSF, human IL-3/GM-CSF, human SIRPA, and human TPO and does not express RAG2 or γ_{c} (referred to herein as MISTRG). In one embodiment, the genetically modified non-human animal of the invention expresses human M-CSF, human IL-3/GM-CSF, human SIRPA, and human TPO, also expresses Fah, and does not express RAG2 or γ_{c} (referred to herein as MISTRG-Fah^{+/+}). In one embodiment, the genetically modified non-human animal of the invention expresses human M-CSF, human IL-3/GM-CSF, human SIRPA, and human TPO, expresses mutant Fah, and does not express RAG2 or γ_{c} (referred to herein as MISTRG-Fah^{+/-}). In one embodiment, the genetically modified non-human animal of the invention expresses human M-CSF, human IL-3/GM-CSF, human TPO, and human SIRPA and does not express Fah and RAG2 or =_{c} (referred to herein as MISTRG-Fah^{-/-}).

In one embodiment, the genetically modified non-human animal of the invention expresses human M-CSF, human IL-3/GM-CSF, human SIRPA, and human TPO, expresses mutant c-kit w41, and does not express RAG2 or γ_{c} (referred to herein as MISTRGW41). In one embodiment, the genetically modified non-human animal of the invention expresses human M-CSF, human IL-3/GM-CSF, human SIRPA, and human TPO, expresses homozygous mutant c-kit w41/w41, and does not express RAG2 or γ_{c} (referred to herein as MISTRG^{w41/w41}). In one embodiment, the genetically modified non-human animal of the invention expresses human M-CSF, human IL-3/GM-CSF, human SIRPA, and human TPO, expresses mutant c-kit w41, and does not express Fah, and does not express RAG2 or γ_{c} (referred to herein as MISTRGW41-Fah^{-/-}). In one embodiment, the genetically modified non-human animal of the invention expresses human M-CSF, human IL-3/GM-CSF, human SIRPA., and human TPO, expresses homozygous mutant c-kit w41/w41, and does not express Fah, and does not express RAG2 or γ_{c} (referred to herein as MISTRG^{w41/w41}Fah^{-/-}).

In one embodiment, the genetically modified non-human animal of the invention expresses human M-CSF, human IL-3/GM-CSF, human SIRPA, and human TPO, expresses heterozygous c-kit mutant, and does not express RAG2 or γ_{c} (referred to herein as MISTRG^{w/+}). In one embodiment, the genetically modified non-human animal of the invention expresses human M-CSF, human IL-3/GM-CSF, human SIRPA, and human TPO, expresses mutant c-kit wv, and does not express RAG2 or γ_{c} (referred to herein as MISTRGckitwv). In one embodiment, the genetically modified non-human animal of the invention expresses human M-CSF, human IL-3/GM-CSF, human SIRPA, and human TPO, expresses heterozygous c-kit mutant, and does not express Fah, and does not express RAG2 or γ_{c} (referred to herein as MISTRG^{w/+}-Fah^{-/-}). In one embodiment, the genetically modified non-human animal of the invention expresses human M-CSF, human IL-3/GM-CSF, human SIRPA, and human TPO, expresses mutant c-kit wv, and does not express Fah, and does not express RAG2 or γ_{c} (referred to herein as MISTRGckitwvFah^{-/-}).

In another particular embodiment, the genetically modified non-human animal of the invention expresses human IL-6, human M-CSF, human IL-3/GM-CSF, human SIRPA, and human TPO and does not express RAG2 or γ_{c} (referred to herein as MISTRG6). In one embodiment, the genetically modified non-human animal of the invention expresses human IL-6, human M-CSF, human IL-3/GM-CSF, human SIRPA, and human TPO, also expresses Fah, and does not express RAG2 or γ_{c} (referred to herein as MISTRG6-Fah^{+/+}). In one embodiment, the genetically modified non-human animal of the invention expresses human IL-6, human M-CSF, human IL-3/GM-CSF, human SIRPA, and human TPO, expresses mutant Fah, and does not express RAG2 or γ_{c} (referred to herein as MISTRG6-Fah^{+/-}). In one embodiment, the genetically modified non-human animal of the invention expresses human IL-6, human M-CSF, human IL-3/GM-CSF, human TPO, and human SIRPA and does not express Fah and RAG2 or γ_{c} (referred to herein as MISTRG6-Fah^{-/-}).

In one embodiment, the genetically modified non-human animal of the invention expresses human IL-6, human M-CSF, human IL-3/GM-CSF, human SIRPA, and human TPO, expresses mutant c-kit w41, and does not express RAG2 or γ_{c} (referred to herein as MISTRG6W41). In one embodiment, the genetically modified non-human animal of the invention expresses human IL-6, human M-CSF, human IL-3/GM-CSF, human SIRPA, and human TPO, expresses homozygous mutant c-kit w41/w41, and does not express RAG2 or γ_{c} (referred to herein as MISTRG6^{w41/w41}). In one embodiment, the genetically modified non-human animal of the invention expresses human IL-6, human M-CSF, human IL-3/GM-CSF, human SIRPA, and human TPO, expresses mutant c-kit w41, and does not express Fah, and does not express RAG2 or γ_{c} (referred to herein as MISTRG6W41-Fah^{-/-}). In one embodiment, the genetically modified non-human animal of the invention expresses human IL-6, human M-CSF, human IL-3/GM-CSF, human SIRPA, and human TPO, expresses homozygous mutant c-kit w41/w41, and does not express Fah, and does not express RAG2 or γ_{c} (referred to herein as MISTRG6^{w41/w″}Fah^{-/-}).

In one embodiment, the genetically modified non-human animal of the invention expresses human IL-6, human M-CSF, human IL-3/GM-CSF, human SIRPA, and human TPO, expresses heterozygous c-kit mutant, and does not express RAG2 or γ_{c} (referred to herein as MISTRG6^{w/+}). In one embodiment, the genetically modified non-human animal of the invention expresses human IL-6, human M-CSF, human IL-3/GM-CSF, human SIRPA, and human TPO, expresses mutant c-kit wv, and does not express RAG2 or γ_{c} (referred to herein as MISTRG6ckitwv). In one embodiment, the genetically modified non-human animal of the invention expresses human IL-6, human M-CSF, human IL-3/GM-CSF, human SIRPA, and human TPO, expresses heterozygous c-kit mutant, and does not express Fah, and does not express RAG2 or γ_{c} (referred to herein as MISTRG6^{w/+}-Fah^{-/-}). In one embodiment, the genetically modified non-human animal of the invention expresses human IL-6, human M-CSF, human IL-3/GM-CSF, human SIRPA, and human TPO, expresses mutant c-kit wv, and does not express Fah, and does not express RAG2 or γ_{c} (referred to herein as MISTRG6ckitwyFah^{-/-}).

In another particular embodiment, the genetically modified non-human animal of the invention expresses human M-CSF, human IL-3/GM-CSF, human SIRPA, human EPO, and human TPO and does not express RAG2 or γ_{c} (referred to herein as MITERGSKI). In one embodiment, the genetically modified non-human animal of the invention expresses human M-CSF, human IL-3/GM-CSF, human SIRPA, human EPO, and human TPO, also expresses Fah, and does not express RAG2 or γ_{c} (referred to herein as MITERGSKI-Fah^{+/+}). In one embodiment, the genetically modified non-human animal of the invention expresses human M-CSF, human IL-3/GM-CSF, human SIRPA, human EPO, and human TPO, expresses mutant Fah, and does not express RAG2 or γ_{c} (referred to herein as MITERGSKI-Fah^{+/-}). In one embodiment, the genetically modified non-human animal of the invention expresses human M-CSF, human IL-3/GM-CSF, human TPO, human EPO, and human SIRPA and does not express Fah and RAG2 or γ_{c} (referred to herein as McIERGSKI-Fah-^{-/-}).

In one embodiment, the genetically modified non-human animal of the invention expresses human M-CSF, human IL-3/GM-CST, human SIRPA, human EPO, and human TPO, expresses mutant c-kit w41, and does not express RAG2 or γ_{c} (referred to herein as MITERGSKIW41). In one embodiment, the genetically modified non-human animal of the invention expresses human M-CSF, human IL-3/GM-CSF, human SIRPA. human EPO, and human TPO, expresses homozygous mutant c-kit w41/w41, and does not express RAG2 or γ_{c} (referred to herein as MITERGSKI^{w41/w41}). In one embodiment, the genetically modified non-human animal of the invention expresses human M-CSF, human IL-3/GM-CST, human SIRPA, human EPO, and human TPO, expresses mutant c-kit w41, and does not express Fah, and does not express RAG2 or γ_{c} (referred to herein as MITERGSKIW41-Fah^{-/-}). In one embodiment, the genetically modified non-human animal of the invention expresses human M-CSF, human IL-3/GN/1-CSF, human SIRPA, human EPO, and human TPO, expresses homozygous mutant c-kit w41/w41, and does not express Fah, and does not express RAG2 or γ_{c} (referred to herein as MITERGSKP^{w41/w41}Fah^{-/-}).

In one embodiment, the genetically modified non-human animal of the invention expresses human M-CSF, human IL-3/GM-CSF, human SIRPA, human EPO, and human TPO, expresses heterozygous c-kit mutant, and does not express RAG2 or γ_{c} (referred to herein as MITERGSKI^{w/+}). In one embodiment, the genetically modified non-human animal of the invention expresses human M-CSF, human IL-3/GM-CSF, human SIRPA, human EPO, and human TITPO, expresses mutant c-kit wv, and does not express RAG2 or γ_{c} (referred to herein as MiTERGSKIckitwv). In one embodiment, the genetically modified non-human animal of the invention expresses human M-CSF, human IL-3/GM-CSF, human SIRPA, human EPO, and human TPO, expresses heterozygous c-kit mutant, and does not express Fah, and does not express RAG2 or γ_{c} (referred to herein as MITERGSKI^{w/+}-Fah^{-/-}). In one embodiment, the genetically modified non-human animal of the invention expresses human M-CSF, human IL-3/GM-CSF, human SIRPA, human EPO, and human TPO, expresses mutant c-kit wv, and does not express Fah, and does not express RAG2 or γ_{c} (referred to herein as MITERGSKIckitwvFah^{-/-}).

In another particular embodiment, the genetically modified non-human animal of the invention expresses human M-CSF, human IL-3/GM-CSF, tg SiRPA, human EPO, and human TPO and does not express RAG2 or γ_{c} (referred to herein as MISTERG). In one embodiment, the genetically modified non-human animal of the invention expresses human M-CSF, human IL-3/GM-CSF, tg SIRPA, human EPO, and human TPO, also expresses Fah, and does not express RAG2 or γ_{c} (referred to herein as MISTERG-Fah^{+/+}). In one embodiment, the genetically modified non-human animal of the invention expresses human M-CSF, human IL-3/GM-CSF, tg SIRPA, human EPO, and human TPO, expresses mutant Fah, and does not express RAG2 or γ_{c} (referred to herein as MISTERG-Fah^{+/-}). In one embodiment, the genetically modified non-human animal of the invention expresses human M-CSF, human IL-3/GM-CSF, human TPO, human EPO, and tg SIRPA and does not express Fah and RAG2 or γ_{c} (referred to herein as MISTERG-Fah^{-/-}).

In one embodiment, the genetically modified non-human animal of the invention expresses human M-CSF, human IL-3/GM-CSF, tg SIRPA, human EPO, and human TPO, expresses mutant c-kit w41, and does not express RAG2 or γ_{c} (referred to herein as MISTERGW41). In one embodiment, the genetically modified non-human animal of the invention expresses human M-CSF, human IL-3/GM-CSF, tg SIRPA, human EPO, and human TPO, expresses homozygous mutant c-kit w41/w41, and does not express RAG2 or γ_{c} (referred to herein as MISTERG^{w41/w41}). In one embodiment, the genetically modified non-human animal of the invention expresses human M-CSF, human IL-3/GM-CSF, tg SIRPA, human EPO, and human TPO, expresses mutant c-kit w41, and does not express Fah, and does not express RAG2 or γ_{c} (referred to herein as MISTERGW41-Fah^{-/-}). In one embodiment, the genetically modified non-human animal of the invention expresses human M-CSF, human IL-3/GM-CSF, tg SIRPA, human EPO, and human TPO, expresses homozygous mutant c-kit w41/w41, and does not express Fah, and does not express RAG2 or γ_{c} (referred to herein as MISTERG^{w41/w41}Fah^{-/-}).

In one embodiment, the genetically modified non-human animal of the invention expresses human M-CSF, human IL-3/GM-CSF, tg SIRPA, human EPO, and human TPO, expresses heterozygous c-kit mutant, and does not. express RAG2 or γ_{c} (referred to herein as MISTERG^{w/+}). In one embodiment, the genetically modified non-human animal of the invention expresses human M-CSF, human IL-3/GM-CSF, tg SIRPA, human EPO, and human TPO, expresses mutant c-kit wv, and does not express RAG2 or γ_{c} (referred to herein as MISTERGekitwv). In one embodiment, the genetically modified non-human animal of the invention expresses human M-CSF, human IL-3/GM-CSF, tg SIRPA, human EPO, and human TPO, expresses heterozygous c-kit mutant, and does not express Fah, and does not express RAG2 or γ_{c} (referred to herein as MISTERG^{w/+}-Fah^{-/-}). In one embodiment, the genetically modified non-human animal of the invention expresses human M-CSF, human IL-3/GM-CSF, tg SIRPA, human EPO, and human TPO, expresses mutant c-kit wv, and does not express Fah, and does not express RAG2 or γ_{c} (referred to herein as MISTERGckitwvFah^{-/-}).

In another particular embodiment, the genetically modified non-human animal of the invention expresses human SIRPA and does not express RAG2 or γ_{c} (referred to herein as RGSKI). In one embodiment, the genetically modified non-human animal of the invention expresses human SIRPA, expresses Fah, and does not express RAG2 or γ_{c} (referred to herein as RGSKI-Fah^{-/-}). In one embodiment, the genetically modified non-human animal of the invention expresses human SIRPA, expresses mutant Fah, and does not express RAG2 or γ_{c} (referred to herein as RGSKI-Fah^{+/-}). In one embodiment, the genetically modified non-human animal of the invention expresses human SIRPA and does not express Fah and RAG2 or γ_{c} (referred to herein as RGSKI-Fah^{-/-}).

In one embodiment, the genetically modified non-human animal of the invention expresses human SIRPA, expresses mutant c-kit w41, and does not express RAG2 or γ_{c} (referred to herein as RGSKIW41). In one embodiment, the genetically modified non-human animal of the invention expresses human SIRPA, expresses homozygous mutant c-kit w41/w41, and does not express RAG2 or γ_{c} (referred to herein as RGSKI^{w41/w41}). In one embodiment, the genetically modified non-human animal of the invention expresses human SIRPA, expresses mutant c-kit w41, and does not express Fah, and does not express RAG2 or γ_{c} (referred to herein as RGSKIW41-Fah^{-/-}). In one embodiment, the genetically modified non-human animal of the invention expresses human SIRPA, expresses homozygous mutant c-kit w41/w41, and does not express Fah, and does not express RAG2 or γ_{c} (referred to herein as RGSKI^{w41/w41}Fah^{-/-}).

In one embodiment, the genetically modified non-human animal of the invention expresses human SIRPA, expresses heterozygous c-kit mutant, and does not express RAG2 or γ_{c} (referred to herein as RGSKI^{w/+}). In one embodiment, the genetically modified non-human animal of the invention expresses human SIRPA, expresses mutant c-kit wv, and does not express RAG2 or γ_{c} (referred to herein as RGSKIckitwv). In one embodiment, the genetically modified non-human animal of the invention expresses human SIRPA, expresses heterozygous c-kit mutant, and does not express Fah, and does not express RAG2 or γ_{c} (referred to herein as RGSKI^{w/+}-Fah^{-/-}). In one embodiment, the genetically modified non-human animal of the invention expresses human SIRPA, expresses mutant c-kit wv, and does not express Fah, and does not express RAG2 or γ_{c} (referred to herein as RGSKIckitwvFah^{-/-}).

In another particular embodiment, the genetically modified non-human animal of the invention expresses human IL-3/GM-CSF, human SIRPA, and human TPO, and does not express RAG2 or γ_{c} (referred to herein as TIRGSKI). In one embodiment, the genetically modified non-human animal of the invention expresses human IL-3/GM-CSF, human SIRPA, and human TPO, also expresses Fah, and does not express RAG2 or γ_{c} (referred to herein as TIRGSKI-Fah^{+/+}). In one embodiment, the genetically modified non-human animal of the invention expresses human IL-3/GM-CSF, human SIRPA, and human TPO, expresses mutant Fah, and does not express RAG2 or γ_{c} (referred to herein as TIRGSKI-Fah^{+/-}). In one embodiment, the genetically modified non-human animal of the invention expresses human IL-3/GM-CSF, human SIRPA, and human TPO and does not express Fah and RAG2 or γ_{c} (referred. to herein as TIRGSKI-Fah^{-/-}).

In one embodiment, the genetically modified non-human animal of the invention expresses human IL-3/GM-CSF, human SIRPA, and human TPO, expresses mutant c-kit w41, and does not express RAG2 or γ_{c} (referred to herein as TIRGSKIW41). In one embodiment, the genetically modified non-human animal of the invention expresses human IL-3/GM-CSF, human SIRPA, and human TPO, expresses homozygous mutant c-kit w41/w41, and does not express RAG2 or γ_{c} (referred to herein as TIRGSKI^{w41/w41}). In one embodiment, the genetically modified non-human animal of the invention expresses human IL-3/GM-CSF, human SIRPA, and human TPO, expresses mutant c-kit w41, and does not express Fah, and does not express RAG2 or γ_{c} (referred to herein as TIRGSKIW41-Fah^{-/-}). In one embodiment, the genetically modified non-human animal of the invention expresses human IL-3/GM-CSF, human SIRPA, and human TPO, expresses homozygous mutant c-kit w41/w41, and does not express Fah, and does not express RAG2 or γ_{c} (referred to herein as TIRGSKI^{w41/w41}Fah^{-/-}).

In one embodiment, the genetically modified non-human animal of the invention expresses human IL-3/GM-CSF, human SIRPA, and human TPO, expresses heterozygous c-kit mutant, and does not express RAG2 or γ_{c} (referred to herein as TIRGSKFI^{w/+}). In one embodiment, the genetically modified non-human animal of the invention expresses human IL-3/GM-CSF, human SIRPA, and human TPO, expresses mutant c-kit wv, and does not express RAG2 or γ_{c} (referred to herein as TIRGSKIekitwv). In one embodiment, the genetically modified non-human animal of the invention expresses human IL-3/GM-CSF, human SIRPA, and human TPO, expresses heterozygous c-kit mutant, and does not express Fah, and does not express RAG2 or γ_{c} (referred to herein as TIRGSKI^{w/+}-Fah^{-/-}). In one embodiment, the genetically modified non-human animal of the invention expresses human IL-3/GM-CSF, human SIRPA, and human TPO, expresses mutant c-kit wv, and does not express Fah, and does not express RAG2 or γ_{c} (referred to herein as TIRGSKIckitwvFah^{-/-}).

In another particular embodiment, the genetically modified non-human animal of the invention expresses human IL-3/GM-CSF, human SIRPA, human EPO, and human TPO and does not express RAG2 or γ_{c} (referred to herein as TIERGSKI). In one embodiment, the genetically modified non-human animal of the invention expresses human IL-3/GM-CSF, human SIRPA, human EPO, and human TPO, also expresses Fah, and does not express RAG2 or γ_{c} (referred to herein as TIERGSKI-Fah^{+/+}). In one embodiment, the genetically modified non-human animal of the invention expresses human IL-3/GM-CSF, human SIRPA, human EPO, and human TPO, expresses mutant Fah, and does not express RAG2 or γ_{c} (referred to herein as TIERGSKI-Fah^{+/-}). In one embodiment, the genetically modified non-human animal of the invention expresses human IL-3/GM-CSF, human TPO, human EPO, and human SIRPA and does not express Fah and RAG2 or γ_{c} (referred to herein as TIERGSKI-Fah^{-/-}).

In one embodiment, the genetically modified non-human animal of the invention expresses human IL-3/GM-CSF, human SIRPA, human EPO, and human TPO, expresses mutant c-kit w41, and does not express RAG2 or γ_{c} (referred to herein as TIERGSKIW41). In one embodiment, the genetically modified non-human animal of the invention expresses human IL-3/GM-CSF, human SIRPA, human EPO, and human TPO, expresses homozygous mutant c-kit w41/w41, and does not express RAG2 or γ_{c} (referred to herein as TIERGSKI^{w41/w41}). In one embodiment, the genetically modified non-human animal of the invention expresses human IL-3/GM-CSF, human SIRPA, human EPO, and human TPO, expresses mutant c-kit w41, and does not express Fah, and does not express RAG2 or γ_{c} (referred to herein as TIERGSKIW41-Fah^{-/-}). In one embodiment, the genetically modified non-human animal of the invention expresses human IL-3/GM-CSF, human SIRPA, human EPO, and human TPO, expresses homozygous mutant c-kit w41/w41, and does not express Fah, and does not express RAG2 or γ_{c} (referred to herein as TIERGSKI^{w41/w41}Fah^{-/-}).

In one embodiment, the genetically modified non-human animal of the invention expresses human IL-3/GM-CSF, human SIRPA, human EPO, and human TPO, expresses heterozygous c-kit mutant, and does not express RAG2 or γ_{c} (referred to herein as TIERGSKI^{w/+}). In one embodiment, the genetically modified non-human animal of the invention expresses human IL-3/GM-CSF, human SIRPA, human EPO, and human TPO, expresses mutant c-kit wv, and does not express RAG2 or γ_{c} (referred to herein as TIERGSKIckitwv). In one embodiment, the genetically modified non-human animal of the invention expresses human IL-3/GM-CSF, human SIRPA, human EPO, and human TPO, expresses heterozygous c-kit mutant, and does not express Fah, and does not express RAG2 or γ_{c} (referred to herein as TIERGSKI^{w/+}Fah^{-/-}). In one embodiment, the genetically modified non-human animal of the invention expresses human IL-3/GM-CSF, human SIRPA, human EPO, and human TPO, expresses mutant c-kit wv, and does not express Fah, and does not express RAG2 or γ_{c} (referred to herein as TIERGSKIckitwvFah^{-/-}).

In one embodiment, the genetically modified non-human animal of the invention is NOD.Cg-Prkde^{scid}I12rg^{tm1Wjl}/SzJ that expresses Fah (referred to herein as NSG-Fah_{+/+}). In one embodiment, the genetically modified non-human animal of the invention is NOD.Cg-Prkdc^{scid}I12rg^{tm1Wjl}/SzJ that expresses mutant Fah (referred to herein as NSG-Fah^{-/-}). In one embodiment, the genetically modified non-human animal of the invention is NOD.Cg-Prkdc^{scid}I12rg^{tm1Wjl}/SzJ that does not express Fah and RAG2 or γ_{c} (referred to herein as NSG-Fah^{-/-}).

In one embodiment, the genetically modified non-human animal of the invention is NOD.Cg-Prkdc^{scid}I12rg^{tm1Wjl}/SzJ that expresses mutant c-kit w41 (referred to herein as NSGW41). In one embodiment, the genetically modified non-human animal of the invention is NOD.Cg-Prkdc^{scid}I12rg^{tm1Wjl}/SzJ that expresses homozygous mutant c-kit w41/w41 (referred to herein as NSG^{w41/w41}). In one embodiment, the genetically modified non-human animal of the invention is NOD.Cg-Prkdc^{scid}I12rg^{tm1Wjl}/SzJ that expresses mutant c-kit w41, and does not express Fah (referred to herein as NSGW41-Fah^{-/-}). In one embodiment, the genetically modified non-human animal of the invention is NOD.Cg-Prkdc^{scid}I12rg^{tm1Wjl}/SzJ that expresses homozygous mutant c-kit w41/w41, and does not express Fah (referred to herein as NSG^{w41/w41}Fah^{-/-}).

In one embodiment, the genetically modified non-human animal of the invention is NOD.Cg-Prkdc^{scid}I12rg^{tm1Wjl}/SzJ that expresses heterozygous c-kit mutant (referred to herein as NSG'^{/+}). In one embodiment, the genetically modified non-human animal of the invention is NOD.Cg-Prkdc^{scid}I12rg^{tm1Wjl}/SzJ that expresses mutant c-kit wv (referred to herein as NSGckitwv). In one embodiment, the genetically modified non-human animal of the invention is NOD.Cg-Prkdc^{scid}I12rg^{tm1Wjl}/SzJ that expresses heterozygous c-kit mutant, and does not express Fah (referred to herein as NSG^{w/+}-Fah^{-/-}). In one embodiment, the genetically modified non-human animal of the invention is NOD.Cg-Prkdc^{I}12rg^{tm1Wjl}/SzJ that expresses mutant c-kit wv, and does not express Fah (referred to herein as NSGckitwvFah^{-/-}).

In some embodiments, the genetically modified non-human animals described herein are engrafted with a human hematopoietic cell. In some embodiments, the genetically modified non-human animals described herein are engrafted with a human liver cell. In some embodiments, the genetically modified non-human animals described herein are engrafted with human liver cells, mouse liver cells, human fetal liver cells, mouse fetal liver cells, human CD14, moue CD14, human CD34, mouse CD34, human CD45, mouse CD45, human CD68, mouse CD68, human CD71, moue CD71, human CD169, mouse CD169, human CD235, moue CD235, human RBC, mouse RBC, human EP, mouse EP, human Hep, mouse Hep, human Albumin, mouse Albumin, human sickle cell, human C3, mouse C3, Ter119, or any combination thereof. In some embodiments, the genetically modified non-human animals described herein are further modified and do not express SRB1^{-/-}. In some embodiments, the genetically modified non-human animals described herein are further modified and do not express SRB2^{-/-}.

In various embodiments, the human hematopoietic cell engrafted, genetically modified non-human animals of the invention are useful for the in vivo evaluation of the growth and differentiation of hematopoietic and immune cells, for the in vivo evaluation of human hematopoiesis, for the in vivo assessment of human erythropoiesis, for the in vivo evaluation of cancer cells, for the in vivo evaluation of diseases associated with deficiencies in the production of red blood cells, for the in vivo evaluation of a treatment of diseases associated with deficiencies in the production of red blood cells, for the in vivo evaluation of diseases associated with genetic deficiencies in the production of red blood cells, for the in vivo evaluation of a treatment of diseases associated with genetic deficiencies in the production of red blood cells, for the in vivo evaluation of human inflammatory diseases in the liver, for the in vivo evaluation of a treatment of human inflammatory disease in the liver, for the in vivo evaluation of fatty liver disease, for the in vivo evaluation of a treatment of fatty liver disease, for the in vivo evaluation of non-alcoholic steatohepatitis, for the in vivo evaluation of a treatment of non-alcoholic steatohepatitis, for the in vivo evaluation of hepatitis, for the in vivo evaluation of a treatment of hepatitis, for the in vivo evaluation of anemia, for the in vivo evaluation of sickle cell disease, for the in vivo evaluation of malaria, for the in vivo evaluation of infection, for the in vivo evaluation of liver diseases, for the in vivo evaluation of a treatment of anemia, for the in vivo evaluation of a treatment of sickle cell disease, for the in vivo evaluation of a treatment of malaria, for the in vivo evaluation of a treatment of infection, for the in vivo evaluation of a treatment of liver diseases, for the in vivo assessment of an immune response, for the in vivo evaluation of vaccines and vaccination regimens, for the use in testing the effect of agents that modulate cancer cell growth or survival, for the in vivo evaluation of a treatment of cancer, for the in vivo production and collection of immune mediators, including human antibodies, and for use in testing the effect of agents that modulate hematopoietic and immune cell function.

### Genetically Modified Non-Human Animals

The invention includes a genetically modified non-human animal that expresses at least one of human M-CSF, human IL-3/GM-CSF, human SIRPA, human TPO, and any combination thereof. In some embodiments, the genetically modified non-human animal comprises a nucleic acid encoding cKit (e.g. cKitw41, cKitVW, etc.) or a mutant thereof, fumarylacetoacetate hydrolase (Fah) or a mutant thereof, or any combination thereof. In one embodiment, the Fall mutant is a deletion of Fah (Fah^{-/-}). In one embodiment, the Fah mutant is a homozygous deletion of Fah (Fah^{-/-}). In one embodiment, the genetically modified non-human animal has a sickle cell disease.

In some embodiments, the genetically modified non-human animal that expresses a human nucleic acid also expresses the corresponding non-human animal nucleic acid. In other embodiments, the genetically modified non-human animal that expresses a human nucleic acid does not also express the corresponding non-human animal nucleic acid. In some embodiments, the genetically modified animal is an animal having one or more genes knocked out to render the animal an immunodeficient animal, as elsewhere described herein. To create a. genetically modified non-human animal, a nucleic acid encoding a human protein can be incorporated into a recombinant expression vector in a form suitable for expression of the human protein in a non-human host cell. In various embodiments, the recombinant expression vector includes one or more regulatory sequences operatively linked to the nucleic acid encoding the human protein in a manner which allows for transcription of the nucleic acid into mRNA and translation of the mRNA into the human protein. The term “regulatory sequence” is art-recognized and intended to include promoters, enhancers and other expression control elements (e.g., polyadenylation signals). Such regulatory sequences are known to those skilled in the art and are described in 1990, Goeddel, Gene Expression Technology: Methods in Enzymology 185, Academic Press, San Diego, Calif. It should be understood that the design of the expression vector may depend on such factors as the choice of the host cell to be transfected and/or the amount of human protein to be expressed.

A genetically modified animal can be created, for example, by introducing a nucleic acid encoding the human protein (typically linked to appropriate regulatory elements, such as a constitutive or tissue-specific enhancer) into an oocyte, e.g., by microinjection, and allowing the oocyte to develop in a female foster animal. Intronic sequences and polyadenylation signals can also be included in the transgene to increase the efficiency of expression of the transgene. Methods for generating genetically modified animals, particularly animals such as mice, have become conventional in the art and are described, for example, in U.S. Pat. Nos. 4,736,866 and 4,870,009 and 1986, Hogan et al. A Laboratory Manual, Cold Spring Harbor, N.Y., Cold Spring Harbor Laboratory. A genetically modified founder animal can be used to breed additional animals carrying the transgene. Genetically modified animals carrying a transgene encoding the human protein of the invention can further be bred to other genetically modified animals carrying other transgenes, or be bred to knockout animals, e.g., a knockout animal that does not express one or more of its genes. In various embodiments, the genetically modified animal of the invention is a mouse, a rat or a rabbit.

In some embodiments, the genetically modified animal of the invention expresses one or more human nucleic acids from the non-human animal's native promoter and native regulatory elements. In some embodiments, the genetically modified animal is a knock-in animal expressing one of more human nucleic acids from the non-human animal's native promoter and native regulatory elements. In other embodiments, the genetically modified animal of the invention expresses a human nucleic acid from the native human promoter and native regulatory elements. The skilled artisan will understand that the genetically modified animal of the invention includes genetically modified animals that express at least one human nucleic acid from any promoter. Examples of promoters useful in the invention include, but are not limited to, DNA pot II promoter, PGK promoter, ubiquitin promoter, albumin promoter, globin promoter, ovalbumin promoter, SV40 early promoter, the Rous sarcoma virus (RSV) promoter, retroviral LTR and lentiviral LTR. Promoter and enhancer expression systems useful in the invention also include inducible and/or tissue-specific expression systems.

In some embodiments, the invention includes genetically modified immunodeficient animals having a genome that includes a nucleic acid encoding a human polypeptide operably linked to a promoter, wherein the animal expresses the encoded human polypeptide. In various embodiments, the invention includes genetically modified immunodeficient non-human animals having a genome that comprises an expression cassette that includes a nucleic acid encoding at least one human polypeptide, wherein the nucleic acid is operably linked to a promoter and a polyadenylation signal and further contains an intron, and wherein the animal expresses the encoded human polypeptide.

In various embodiments, various methods are used to introduce a human nucleic acid sequence into an immunodeficient animal to produce a genetically modified immunodeficient animal that expresses a human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and ichiki (2006, Genetically Engineered Mice Handbook, CRC Press), Holker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol 13iol., Humana Press), Meyer et al. (2010, Proc. Nat. Acad. Sci. USA 107:15022-15026), and Gibson (2004, A Primer Of Genome Science 2^{nd} ed, Sunderland, Massachusetts: Sinauer), U.S. Pat. No. 6,586,251, Rathinam et al, (2011, Blood 118:3119-28), Willinger et al., (2011, Proc Natl Acad Sci USA, 108:2390-2395), Rongvaux et al., (2011, Proc Nail Acad Sci USA, 108:2378-83) and Valenzuela et al. (2003, Nat Biot 21:652-659).

In some embodiments, the compositions and methods of the invention comprise genetically modified immunodeficient animals deficient in B cell and/or T cell number and/or function, alone, or in combination with a deficiency in NK cell number and/or function (for example, due to an IL2 receptor gamma chain deficiency (i.e., γ_{c}^{-/-})), and having a genome that comprises a human nucleic acid operably linked to a promoter, wherein the animal expresses the encoded human polypeptide. The generation of the genetically modified animal of the invention can be achieved by methods such as DNA injection of an expression construct into a preimplantation embryo or by use of stem cells, such as embryonic stem (ES) cells or induced pluripotent stem (iPS)

In one embodiment, the human nucleic acid is expressed by the native regulatory elements of the human gene. In other embodiments, the human nucleic acid is expressed by the native regulatory elements of the non-human animal. In other embodiments, human nucleic acid is expressed from a ubiquitous promoter. Nonlimiting examples of ubiquitous promoters useful in the expression construct of the compositions and methods of the invention include, a 3-phosphoglycerate kinase (PGK-1) promoter, a beta-actin promoter, a ROSA26 promoter, a heat shock protein 70 (Hsp70) promoter, an EF-1 alpha gene encoding elongation factor 1 alpha (EF1) promoter, an eukaryotic initiation factor 4A (eIF-4A1) promoter, a chloramphenicol acetyltransferase (CAT) promoter and a CMV (cytomegalovirus) promoter.

In other embodiments, the human nucleic acid is expressed from a tissue-specific promoter. Nonlimiting examples of tissue-specific promoters useful in the expression construct of the compositions and methods of the invention include a promoter of a gene expressed in the hematopoietic system, such as a M-CSF promoter, an IL-3 promoter, a GM-CST promoter, a SIR.PA promoter, a TPO promoter, an IFN-β promoter, a Wiskott-Aldrich syndrome protein (WASP) promoter, a CD34 promoter, a CD45 (also called leukocyte common antigen) promoter, a CD71 promoter, a CD169 promoter, a Flt-1 promoter, an endoglin (CD105) promoter and an ICAM-2 (Intracellular Adhesion Molecule 2) promoter. These and other promoters useful in the compositions and methods of the invention are known in the art as exemplified in Abboud et al. (2003, J. Histochem & Cytochem. 51:941-949), Schorpp et al. (1996, NAR 24:1787-1788), McBurney et al. (1994, Devel. Dynamics, 200:278-293) and Majumder et al. (1996, Blood 87:3203-3211). Further to comprising a promoter, one or more additional regulatory elements, such as an enhancer element or intron sequence, is included in various embodiments of the invention. Examples of enhancers useful in the compositions and methods of the invention include, but are not limited to, a cytomegalovirus (CMV) early enhancer element and an SV40 enhancer element. Examples of intron sequences useful in the compositions and methods of the invention include, but are not limited to, the beta globin intron or a generic intron. Other additional regulatory elements useful in some embodiments of the invention include, but are not limited to, a transcription termination sequence and an mRNA polyadenylation (pA) sequence.

In some embodiments, the methods of introduction of the human nucleic acid expression construct into a preimplantation embryo include linearization of the expression construct before it is injected into a preimplantation embryo. In some embodiments, the expression construct is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

In other embodiments, the expression construct may be transfected into stem cells (ES cells or iPS cells) using well-known methods, such as electroporation, calcium-phosphate precipitation and lipofection. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be microinjected into preimplantation embryos. For a detailed description of methods known in the art usefUl for the compositions and methods of the invention, see Nagy et al., (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

The genetically modified non-human animals of the invention can be crossed to immunodeficient animal to create an immunodeficient animal expressing at least one human nucleic acid. Various embodiments of the invention provide genetically modified animals that include a human nucleic acid in substantially all of their cells, as well as genetically modified animals that include a human nucleic acid in some, but not all their cells. One or multiple copies, adjacent or distant to one another, of the human nucleic acid may be integrated into the genome of the cells of the genetically modified animals.

In some embodiments, the invention is a genetically modified non-human mouse engrafted with at least one human hematopoietic cell. In some embodiments, the invention is a genetically modified non-human mouse engrafted with at least one human liver cell. In some embodiments, the invention is a genetically modified non-human mouse engrafted with at least one human spleen cell. In other embodiments, the invention is a method of engrafting human hematopoietic cells in a genetically modified non-human animal. In other embodiments, the invention is a method of engrafting human liver cells in a genetically modified non-human animal. In other embodiments, the invention is a method of engrafting human spleen cells in a genetically modified non-human animal. The engrafted human heniatopoietic cells useful in the compositions and methods of the invention include any human hematopoietic cell. Non-limiting examples of human hematopoietic cells useful in the invention include, but are not limited to, HSC, HSPC, leukemia initiating cells (LIC), and hematopoietic cells of any lineage at any stage of differentiation, including terminally differentiated hematopoietic cells of any lineage. Such hematopoietic cells can be derived from any tissue or location of a human donor, including, but not limited to, bone marrow, peripheral blood, liver, fetal liver, or umbilical cord blood. Such hematopoietic cells can be isolated from any human donor, including healthy donors, as well as donors with disease, such as cancer, including leukemia.

In other embodiments, the invention is a method of engrafting human hematopoietic cells in a genetically modified non-human animal. In some embodiments, the genetically modified non-human animal into which human hematopoietic cells are engrafted is an immunodeficient animal. Engraftment of hematopoietic cells in the genetically modified animal of the invention is characterized by the presence of human hematopoietic cells in the engrafted animal In particular embodiments, engraftment of hematopoietic cells in an immunodeficient animal is characterized by the presence of differentiated human heniatopoietic cells in the engrafted animal in which hematopoietic cells are provided, as compared with appropriate control animals.

In other embodiments, the invention is a method of engrafting human liver cells in a genetically modified non-human animal in some embodiments, the genetically modified non-human animal into which human liver cells are engrafted is an immunodeficient animal. Engraftment of liver cells in the genetically modified animal of the invention is characterized by the presence of human liver cells in the engrafted animal. In particular embodiments, engrafiment of liver cells in an immunodeficient animal is characterized by the presence of differentiated human liver cells in the engrafted animal in which liver cells are provided, as compared with appropriate control animals.

In other embodiments, the invention is a method of engrafting human spleen cells in a genetically modified non-human animal. In some embodiments, the genetically modified non-human animal into which human spleen cells are engrafted is an inumunodeficient animal Engraftment of spleen cells in the genetically modified animal of the invention is characterized by the presence of human spleen cells in the engrafted animal. In particular embodiments, engratiment of spleen cells in an immunodeficient animal is characterized by the presence of differentiated human spleen cells in the engrafted animal in which spleen cells are provided, as compared with appropriate control animals.

The genetically modified non-human animals provided in various embodiments of the present invention have various utilities such as, but not limited to, for use as models of growth and differentiation of hematopoietic cells, for use as models of growth and differentiation of liver cells, tor use as models of growth and differentiation of spleen cells, for the in vivo evaluation of human hematopoiesis, for the in vivo assessment of human erythropoiesis, for the in vivo evaluation of cancer cells, for the in vivo evaluation of diseases associated with deficiencies in the production of red blood cells, for the in vivo evaluation of a treatment of diseases associated with deficiencies in the production of red blood cells, for the in vivo evaluation of diseases associated with genetic deficiencies in the production of red blood cells, for the in vivo evaluation of a treatment of diseases associated with genetic deficiencies in the production of red blood cells, for the in vivo evaluation of human inflammatory diseases in the liver, for the in vivo evaluation of a treatment of human inflammatory disease in the liver, for the in vivo evaluation of fatty liver disease, for the in vivo evaluation of a treatment of fatty liver disease, for the in vivo evaluation of non-alcoholic steatohepatitis, for the in vivo evaluation of a treatment of non-alcoholic steatohepatitis, for the in vivo evaluation of hepatitis, for the in vivo evaluation of a treatment of hepatitis, for the in vivo evaluation of anemia, for the in vivo evaluation of sickle cell disease, for the in vivo evaluation of malaria, for the in vivo evaluation of invention, for the in vivo evaluation of liver diseases, for the in vivo evaluation of a treatment of anemia, for the in vivo evaluation of a treatment of sickle cell disease, for the in vivo evaluation of a treatment of malaria, for the in vivo evaluation of a treatment of infection, for the in vivo evaluation of a treatment of liver diseases, for in vivo study of an immune response, for in vivo evaluation of vaccines and vaccination regimens, for the use in testing the effect of agents that modulate cancer cell growth or survival, for the in vivo evaluation of a treatment of cancer, for in vivo production and collection of immune mediators, such as an antibody, and for use in testing the effect of agents that affect hematopoietic and immune cell function.

Engraftment of human cells, such as human hematopoietic cells, in genetically modified and/or immunodeficient non-human animals has traditionally required conditioning prior to administration of the hematopoietic cells, either sub-lethal irradiation of the recipient animal with high frequency electromagnetic radiation, generally using gamma or X-ray radiation, or treatment with a radiomimetic drug such as busulfan or nitrogen mustard. Conditioning is believed to reduce numbers of host hematopoietic cells, create appropriate microenvironmental factors for engraftment of human hematopoietic cells, and/or create microenvironmental niches for engraftment of human hematopoietic cells. Standard methods for conditioning are known in the art, such as described herein and in J. Hayakawa et al, 2009, Stem Cells, 2(1):175-182. Methods for engraftment of human cells, such as human hematopoietic cells, in immunodeficient an are provided according to embodiments of the present invention Which include providing human cells, such as human hematopoietic cells, to the immunodeficient animals, with or without irradiating the animals prior to administration of the hematopoietic cells. Methods for engraftment of human hematopoietic cells in inununodeficient animals are provided according to embodiments of the present invention which include providing human hematopoietic cells to the genetically modified non-human animals of the invention, with or without, administering a radiomimetic such as busulfan or nitrogen mustard, to the animals prior to administration of the hematopoietic cells.

In some embodiments, the methods of h.ematopoietic cell engraftment in a genetically modified non-human animal according to embodiments of the present invention include providing human hematopoietic cells to a genetically modified animal of the invention as elsewhere described here. In some embodiments, the genetically^{,} modified non-human animal of the invention is an immunodeficient animal that deficient in non-human B cell number and/or function, non-human T cell number and/or function, and/or non-human NK cell number and/or function. In other embodiments, the immunodeficient animal has severe combined immune deficiency (SCID). SCID refers to a condition characterized by the absence of T cells and lack of B cell function. Examples of SCID include: X-linked SCID, which is characterized by gamma chain gene mutations in the IL2RG gene and the lymphocyte phenotype T(−) B(+) NK(−); and autosomal recessive SCID characterized by Jak3 gene mutations and the lymphocyte phenotype T(−) B(+) NK(−), ADA gene mutations and the lymphocyte phenotype T(−) B(−) NK(−), IL-7R alpha-chain mutations and the lymphocyte phenotype T(−) B(+) NK(+), CD3 delta or epsilon mutations and the lymphocyte phenotype T(−) B(+) NK(+), RAG1/RAG2 mutations and the lymphocyte phenotype T(−) B(−) NK(+), Artemis gene mutations and the lymphocyte phenotype T(−) B(−) NK(+), CD45 gene mutations and the lymphocyte phenotype T(−) B(+) NK(+).

In some embodiments, the methods of h.ematopoietic cell engraftment in a genetically modified animal according to embodiments of the present invention include providing human hematopoietic cell to in a genetically modified non-human animal having the severe combined immunodeficiency mutation (Prkdc^{scid}), commonly referred to as the scid mutation. The scid mutation is well-known and located on mouse chromosome 16 as described in Bosma et al. (1989, Immunogenetics 29:54-56). Mice homozygous for the scid mutation are characterized by an absence of functional T cells and B cells, lymphopenia, hypoglobulinemia and a normal hematopoietic microenvironment. The scid mutation can be detected, for example, by detection of markers of the scid mutation using well-known methods.

In other embodiments, the methods of hematopoietic cell engraftment in a genetically modified animal according to embodiments of the present invention include providing human hematopoietic cells to genetically modified immunodeficient non-human animal having an IL2 receptor gamma chain deficiency, either alone, or in combination with, the severe combined immunodeficiency (scid) mutation. The term “IL2 receptor gamma chain deficiency” refers to decreased IL2 receptor gamma chain. Decreased IL2 receptor gamma chain can be due to gene deletion or mutation. Decreased IL2 receptor gamma chain can be detected, for example, by detection of IL2 receptor gamma chain gene deletion or mutation and/or detection of decreased IL2 receptor gamma chain expression using well-known methods.

In addition to the naturally occurring human nucleic acid and amino acid sequences, the term encompasses variants of human nucleic acid and amino acid sequences As used herein, the term “variant” defines either an isolated naturally occurring genetic mutant of a human or a recombinantly prepared variation of a human, each of which contain one or more mutations compared with the corresponding wild-type human. For example, such mutations can be one or more amino acid substitutions, additions, and/or deletions. The term “variant” also includes non-human orthologues. In some embodiments, a variant polypeptide of the present invention has at least 90%, 91%, 92%, 93%, 94%, 95%, 96%, 97%, 98% or 99% identity to a wild-type human polypeptide.

The percent identity between two sequences is determined using techniques as those described elsewhere herein. Mutations can be introduced using standard molecular biology techniques, such as site-directed mutagenesis and PCR-mediated mutagenesis. One of skill in the art will recognize that one or more amino acid mutations can be introduced without altering the functional properties of human proteins.

Conservative amino acid substitutions can he made in human proteins to produce human protein variants. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

Human variants can include synthetic amino acid analogs, amino acid derivatives and/or non-standard amino acids, illustratively including, without limitation, alpha-aminobutyric acid, citrulline, canavanine, cyanoatanine, diaminobutyric acid, diaminopimelic acid, dihydroxy-phenylalanine, djenkolic acid, homoarginine, hydroxyproline, norleucine, norvaline, 3-phosphoserinc, homoserine, 5-hydroxytryptophan, 1-methylhistidine, methylhistidine, and ornithine.

Human variants are encoded by nucleic acids having a high degree of identity with a nucleic acid encoding a wild-type human. The complement of a nucleic acid encoding a human variant specifically hybridizes with a nucleic acid encoding a wild-type human under high stringency conditions.

The term “nucleic acid” refers to RNA or DNA molecules having more than one nucleotide in any form including single-stranded, double-stranded, oligonucieotide or polynucleotide. The term “nucleotide sequence” refers to the ordering of nucleotides in an oligonucleotide: or polynucleotide in a single-stranded form of nucleic acid.

Nucleic acids encoding a human variant can be isolated or generated recombinantly or synthetically using well-known methodology.

Isolation of human hematopoietic cells, administration of the human hematopoietic cells to a host animal and methods for assessing engraftment thereof are well-known in the art. Hematopoietic cells for ad .ministration to host animal can be obtained from any tissue containing hematopoietic cells such as, but not limited to, umbilical cord blood, bone marrow, peripheral blood, cytokine or chemotherapy-mobilized peripheral blood and fetal liver. Hematopoietic cells can be administered into newborn or adult animals by administration via various routes, such as, but not limited to, intravenous, intrahepatic, intraperitoneal, intrafemoral andlor intratibial. Engraftment of human hematopoietic cells in the genetically modified animal of the invention can be assessed by any of various methods, such as, but not limited to, flow cytometric analysis of cells in the animals to which the human hematopoietic cells are administered at one or more time points following the administration of hematopoietic cells.

Exemplary methods of isolating human hematopoietic cells, of administering human hematopoietic cells to a host animal, and of assessing engraftment of the human hematopoietic cells in the host animal are described herein and in Pearson et al, (2008, Curr. Protoc. 81:1-15), Ito et al. (2002, Blood 100:3175-3182), Traggiai et at. (2004, Science 304:104-107), Ishikawa et al, (2005, Blood 106:1565-1573), Shultz et al. (2005, J. immunol. 174:6477-6489) and Holyoake et al. (1999, Exp Hematol. 27:1418-27).

In some embodiments of the invention, the human hematopoietic cells are isolated from an original source material to obtain a population of cells enriched for a particular hematopoietic cell population (e.g., FISCs, HShCs LICs, cD34+, CD34−, lineage specific marker, etc.). The isolated hematopoietic cells may or may not be a pure population. In one embodiment, hematopoietic cells useful in the compositions and methods of the invention are depleted of cells having a particular marker, such as CD34. In another embodiment, hematopoietic cells useful in the compositions and methods of the invention are enriched by selection for a marker, such as CD34. In some embodiments, hematopoietic cells useful in the compositions and methods of the invention are a population of cells in which CD34+ cells constitute about 1-100% of the cells, although in certain embodiments, a population of cells in which CD34+ cells constitute fewer than 1% of total cells can also be used. In certain embodiments, the hematopoietic cells useful in the compositions and methods of the invention are a T cell-depleted population of cells in which CD34+ cells make up about 1-3% of total cells, a lineage-depleted population of cells in which CD34+ cells make up about 50% of total cells, or a CD34+positive selected population of cells in which CD34+ cells make up about 90% of total cells.

The number of hematopoietic cells administered is not considered limiting with regard to the generation of a human h.ematopoietic and/or immune system in a genetically modified non-human animal expressing at least one human gene. Thus, by way of non-limiting example, the number of h.ematopoietic cells administered can range from about 1×10³ to about 1×10⁷, although in various embodiments, more or fewer can also be used. By way of another non-limiting example, the number of FISPCs administered can range from about 3×10³ to about b 1×10° CD34+ cells when the recipient is a mouse, although in various embodiments, more or fewer can also be used. For other species of recipient, the number of cells that need to be administered can be determined using only routine experimentation.

Generally, engraftment can be considered successful when the number (or percentage) of human hematopoietic cells present in the genetically modified non-human animal is greater than the number (or percentage) of human cells that were administered to the non-human animal, at a point in time beyond the lifespan of the administered human hematopoietic cells. Detection of the progeny of the administered hematopoietic cells can be achieved by detection of human DNA in the recipient animal, for example, or by detection of intact human hematopoietic cells, such as by the detection of the human cell surface marker, such as CD45 for example. Serial transfer of human hematopoietic cells from a first recipient into a secondary recipient, and engraftment of human hematopoietic cells in the second recipient, is a further optional test of engraftment in the primary recipient, Engraftment can be detected by flow cytometry as 0.05% or greater human CD45+ cells in the blood, spleen or bone marrow at 1-4 months after administration of the human hematopoietic cells. A cytokine (e.g., GM-CSF) can be used to mobilize stem cells, tor example, as described in Watanabe (1997, Bone Marrow Transplantation 19:1175-1181).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and arc not to be construed as limiting in any way the remainder of the disclosure.

### Example 1

### Evaluation of Human RRCs in MISTERG-FAH^{-/-} Mouse Model

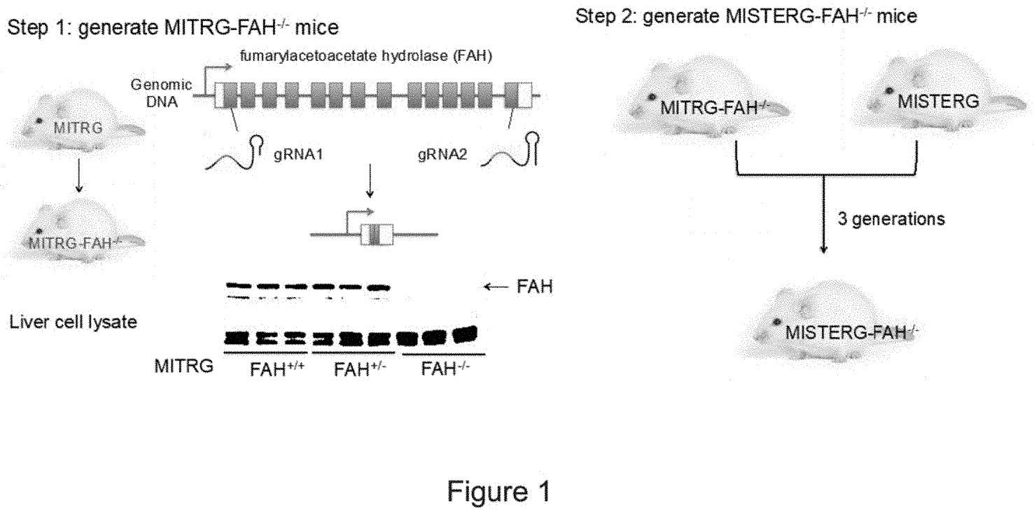

Currently, there is no humanized model of human liver to study e.g. liver disease. A human haematopoietic system (HS) in mice produces most HS cells but the mouse liver eliminates red blood cell granulocytes and platelets. Thus, using the CRISPR-Cas9-based technology (FIG. 1), the MITRG-FAH^{-/-} mice were engrafted by deleting the FAH coding gene in MISTRG mice. Subsequent expansion of MISTERG-FAH^{-/-} colony has also been then generated.

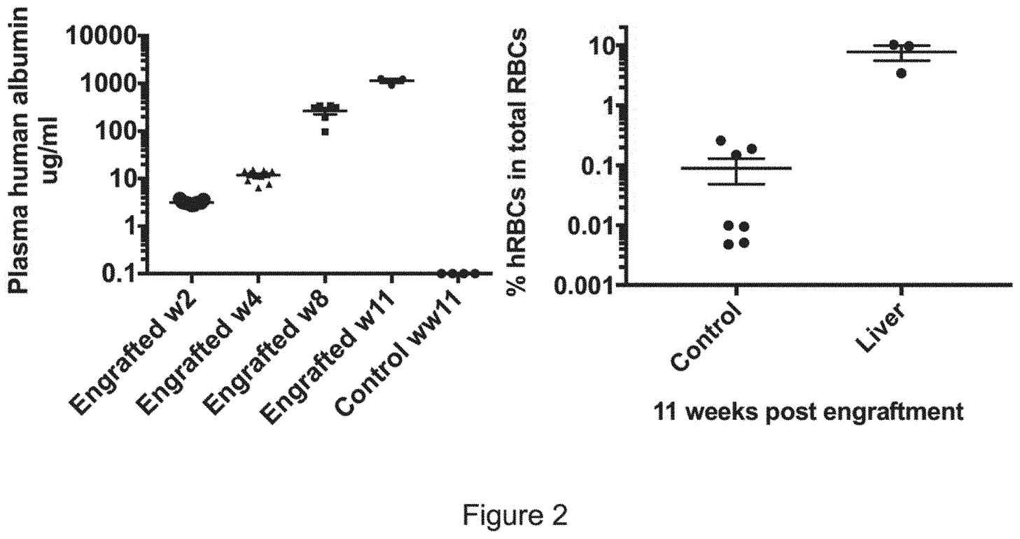

Human primary hepatocyte engraftment in MISTERG-FAH^{-/-} mice was then validated, Human primary hepatocytes were purchased from ThermoFisher. The engraftment of human hepatocytes was validated by measuring serum human albumin levels. In the first animal cohort, as shown in FIG. 2, repopulation of human hepatocytes was achieved in MISTERG-FAH^{-/-} mice. Serum human albumin concentration continued to increase over time. More importantly, the frequency of peripheral human RBCs in MISTERG-FAH^{-/-} mice can reach as high as 10% 11 weeks post engraftment without clodronate treatment. Liver humanization apparently abolishes the sequestration of human RBCs in the liver. Therefore, it is not necessary to cross MISTERG-FAH^{-/-} mice with MISTERG-SRB^{-/-} mice. Besides, the MISTERG-FAH^{-/-} mouse model does not need ciodronate treatment, which should dramatically increase the lifespan of the animals. Thus, the MISTERG-FAH^{-/-} mouse model is ideal for both liver and blood stage infection with P. vivax and P. falciparum.

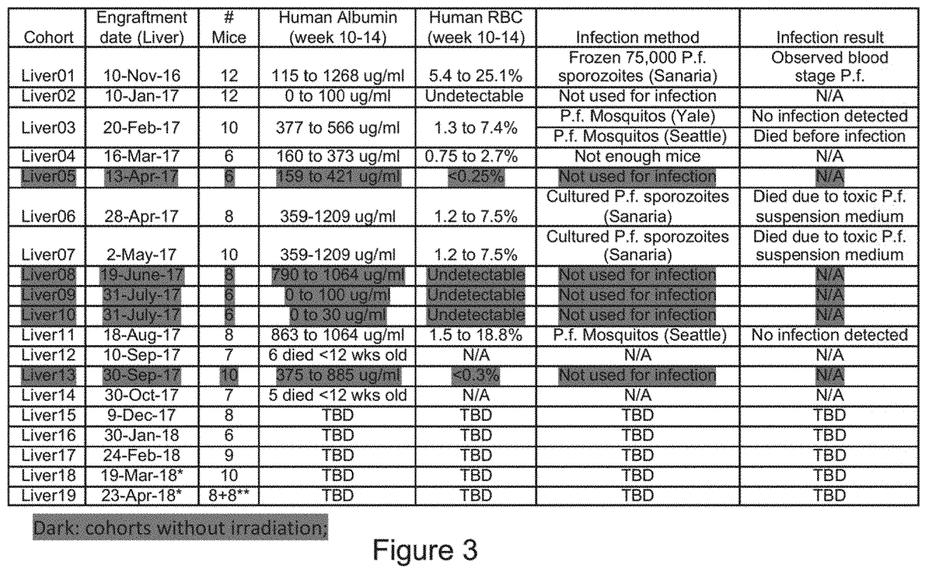

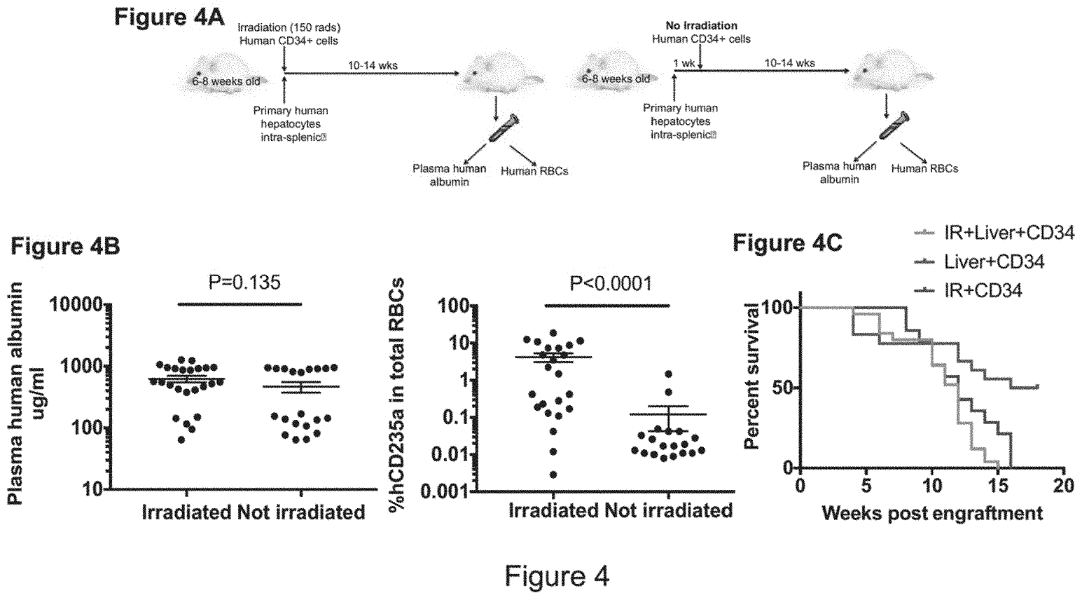

As shown in FIG. 3, 17 MISTERG-FAH^{-/-} cohorts were generated, and plasma human albumin and peripheral human RBC levels in these animals were evaluated. In the first several cohorts, MISTERG-FAH^{-/-} were irradiated (150 rads) and engrafted with 50,000 fetal liver or cord blood CD34+ cells by i.v. injection, and then engrafted with 1-2 million primary human hepatocytes by intra-splenic injection (FIG. 4A). Using this irradiation strategy, the majority of the animals were successfully engrafted with both human hepatocytes and peripheral RBCs (FIG. 4B), However, the engrafted. MISTERG-FAH^{-/-} mice developed anemia starting at week 8. The majority of the animals died between week 11 and 15 (FIG. 4C). Anemia is common in all MISTRG family mouse models, largely due to the reconstitution of human myeloid cells that destroyed mouse RBCs. The anemia can be alleviated if no irradiation is performed at the time of CD34 engraftment. Therefore, two different strategies were used to produce M.ISTERG-FAH^{-/-} cohorts (FIG. 4A) either with or without irradiation. The majority of the unirradiated MISTERG-FAH^{-/-} mice engrafted with human hepatocytes and CD34+ cells survived for at least 15 weeks (FIG. 4C). However, without pre-conditioning by irradiation, human RBC reconstitution in these cohorts was severely impaired (FIG. 4B).

The lifespan of MISTERG-FAH^{-/-} mice is a major limitation for the liver/blood dual stage infection, which was no different from the commonly engrafted MISTRG family mice, suggesting anemia still contributes substantially to the shortened lifespan. No irradiation of the MISTERG-FAH^{-/-} mice improves longevity significantly, but is not ideal for RBC development, probably due to lack of bone marrow niches for human erythropoiesis without pre-conditioning. Therefore models that do not require irradiation were considered. Thus, an ideal candidate for this is the cKit mutant mouse.

The cKitw41 or cKitWV mutation impairs the binding of stem cell factor (SCF) to its ligand cKit on mouse hematopoietic progenitor cells including erythroid progenitor cell. With diminished host hematopoiesis, mice with cKitw41 or cKitWV mutation have been successfully engrafted with human CD34+ cells without irradiation. cKitw41 or cKitWV mutation were introduced into the MISTRG background. Human erythropoiesis in non-irradiated MISTRG-cKitwv mice is more efficient than MISTRG mice,

The process of generating MISTRG-FAH^{-/-} cKitw41 and MISTRG-FAH^{-/-} cKitwv mice is now evaluated because these mouse models are ideal for the liver/blood dual stage infection.

### Example 2

### A Mouse Model of Human Erythropoiesis which Combines Human Liver and a Human Immune System

Human Hem.a.topoietic Stem Cell Engraftment in Humanized Immunodeficient mice

This example focuses on the development of a pre-clinical mouse model for the study of primary human sickle cell disease (SCD) and its sequelae. The first ever data showing full maturation, enucleation and circulation of human red blood cells (huRBC) in vivo in the murine host is provided. Fully characterized and optimized HuRedCell (MISTRG-FAH^{-/-} engrafted with human RBC) and HuSickieCell (MISTRG-FAH^{-/-} engrafted with human sickle cells) mice with the ultimate goal to harness this powerful model br the development of novel treatments for SCD and other RBC disorders was then examined.

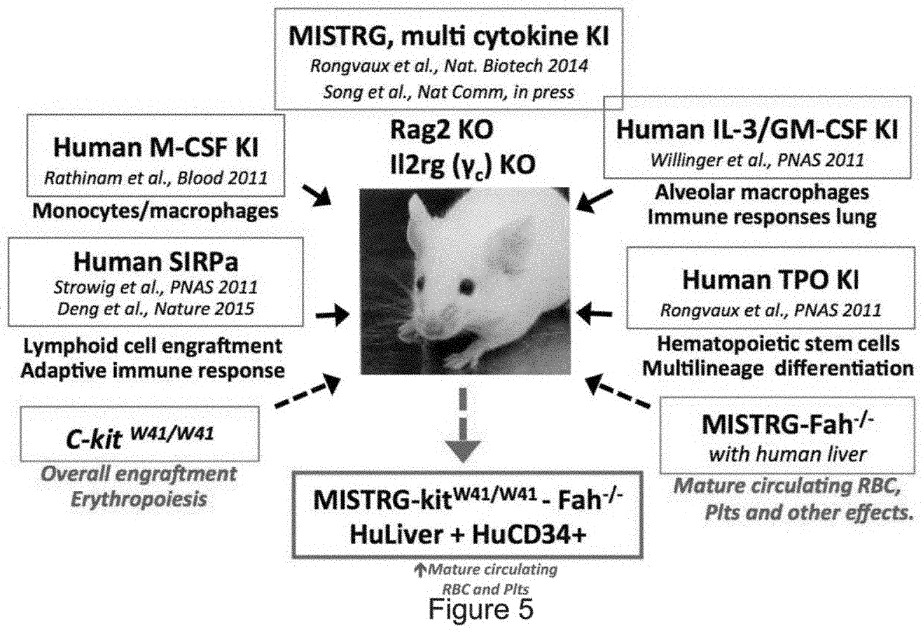

The murine host has remained a readily available and ethically acceptable model for the study of human diseases and therapeutic testing. Immunodeficient mouse models support engraftment of human hematopoietic stem cells (HSC) but with limitation in efficiency and mature lineage representation. Systematic genetic alterations of the immunodeficient host have generated progressively more permissive and supportive marine hosts (Goyama S et al., 2015, Blood, 125:2630). The most frequently used immunodeficient mouse strains lack T-, B-, and NK-cells and have defective dendritic cell function, NOD.Cg-Prkdc^{scid}I12rg^{tm1Wjl}/SzJ (Nod Scid gamma (NSG)) mice in addition carry a polymorphism in the Sirpa gene, expressed on murine macrophages, that allows enhanced binding to the human CD47 ligand, providing a “don't-eat-me” signal (Takenaka K et al., 2007, Nat Immunol., 8:1313-1323). Human SIRPA (5), introduced as transgene or knockin, e,g. into the Rag^{-/-}I12rγ^{-/-} (RG) background (SRG), replicates this effect and significantly improves engraftment of human FISC (Strowig T et al., 2011, Proc Natl Acad Sci USA, 108:13218-13223). Combined knockin of several non-crossreactive human cytokines (M-CSF, IL3/GM-CSF, and Thrombopoietin) into the corresponding murine loci in the SRG strain, in short termed “MISTRG”, has enhanced engraftment and maintenance of human EISCs with multi-lineage differentiation (FIG. 5) (Rongvaux A et al., 2013, Annu Rev Immunol., 31:635-674; Deng K et al., 2015, Nature, 517:381-385; Saito Y et al., 2016, Blood, 128:1829-1833; Theocharides AP et al., Flaematologica, 2016, 101:5-19; Song Y et al., 2019, Nature Communications). The lack of the corresponding murine cytokines in MISTRG mice compromises murine stem cell retention in the niche, which allows human HSC to engraft without irradiation and its accompanying morbidities. This is even more pronounced in mice that carr mutations of the murine stem cell factor receptor c-kit (Waskow C et al., 2009, Nature Methods, 6:267-269; Cosgun KN et al., 2014, Cell Stem Cell, 15:227-238).

### Erythropoiesis and Erythroblastic Islands

Erythroblastic islands (Manwani D et al., 2008, Curr Top Dcv Biot., 82:23-53): Macrophages (MΦ) play a central role in erythropoiesis. They store excess iron in the form of ferritin, a key source of iron during erythropoiesis. They provide proliferative signals, such as insulin-like growth factor-1 (IGF1) and bone morphogenetic protein 4 (BMP4), to the earliest erythroid progenitors. Via direct cell-cell contacts in erythroblastic islands (EBIs) mediated by adhesion molecules on MΦs (EMP, VCAM1, uV, CD163, CD169) and on erythroblasts (ICAM4, EMP, A4β1) direct cues drive terminal differentiation. CD169 has been recognized to mark the central EBI MΦ with distinct functions from CD169-MΦ (Chow A et al., 2013, Nat Med. 2013, 19:429-436; Seu K G et al., 2017, Frontiers in Immunology, 8:1140). In addition to their pro-erythroid role in the bone marrow, MΦ in the spleen and liver repair damaged RBC and eventually take senescent RBCs out of circulation (Klei TR et al., 2017, Frontiers in immunology, 8:73). Until recently, immunodeficient mouse models have been poor hosts for human erythropoiesis. MISTRG mice not only afford higher overall engraftment but also show significantly higher erythroid lineage representation in the bone marrow and not only from IJCB (Song Y et al., 2019, Nature Communications), but also from adult BM derived CD34+ cells (FIG. 5 and Song Y et al., 2019, Nature Communications). Introduction of a c-kit mutation into MISTRG mice further improves multi-lineage hematopoiesis without the need for toxic conditioning thereby eliminating damage to the microenvironment and the host milieu.

### RBC Destruction by the Murine Innate Immune System

Despite robust HSC engraftment and myelo- and erythropoiesis in BM, all humanized immunodeficient mouse models lack mature human RBC, platelets, and other cells in peripheral blood (Yurino A et al., 2016, Stem Cell Reports, 7:425-438; Rahmig S et al., 2016, Stem Cell Reports, 7:591-601). With universal absence of a murine adaptive immune system the culprit is likely the innate immune system.

Phagocytic tolerance is achieved in part by engagement of the SIRPα receptor on MΦs by the ubiquitously expressed CD47, leading to the delivery of a don't-eat-me signal While humanization of the SIRPA gene has led to enhanced overall engraftment it appears to be insufficient to protect cells in circulation. Additional phagocytic tolerance can be achieved by eliminating recipient phagocytic cells altogether by application of clodronate-containing liposomes (Yurino A et al., 2016, Stem Cell Reports, 7:425-438; Rahmig S et al., 2016, Stem Cell Reports, 7:591-601). Yet liposomal clodronate treatment is toxic with significant mortality and abrogates niurine and human phagocytes with only temporary benefit due to regeneration of phagocytes within <1-2 weeks. A recent study shows that complement mediates adherence of human RBC to mouse phagocytes, yet in vivo depletion of complement does not improve RBC survival, unless phagocytes are concurrently abrogated by clodronate treatment (Chen B et at., 2017, Stem Cell Reports, 9:1034-1042).

A solution is needed that modulates specifically murine phagocyte-huRBC interactions, that does not cause undue toxicity, and that is long-lived.

SCD is one of the oldest inherited genetic diseases based on a single point mutation in the β-Tobin gene. SCD carries devastating multi-organ consequences during the life-time of the individual with significant reduction in life-expectancy. Yet, SCD is not one disease and its clinical manifestations and severity are determined by numerous intrinsic (erythropoietic) and extrinsic (host) modifiers. Very few treatments for SCD exist and the paucity of clinical trials is evidence for the dire need for new approaches. SCD mouse models have “fixed” genetics with introduction of the human β^{6Glu-Val} mutant globin, either without the γ-globin genes or with fixed γ-globin expression, into the mouse genome in the context of otherwise murine RBC. Thus, the first humanized mouse model of SCD is described herein. Ultimately, this present model is ideally suited for the study of all hemoglobinopathies and diseases of the red blood cell.

### Adult HSPC Engraftment and Erythropoiesis

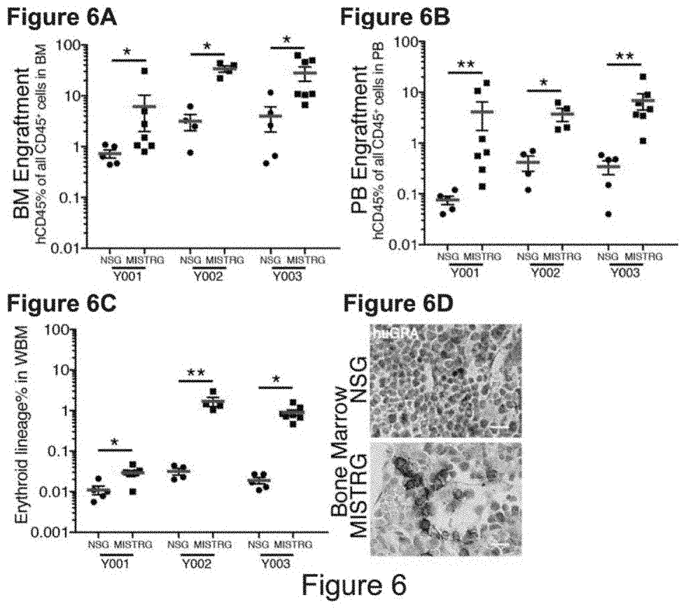

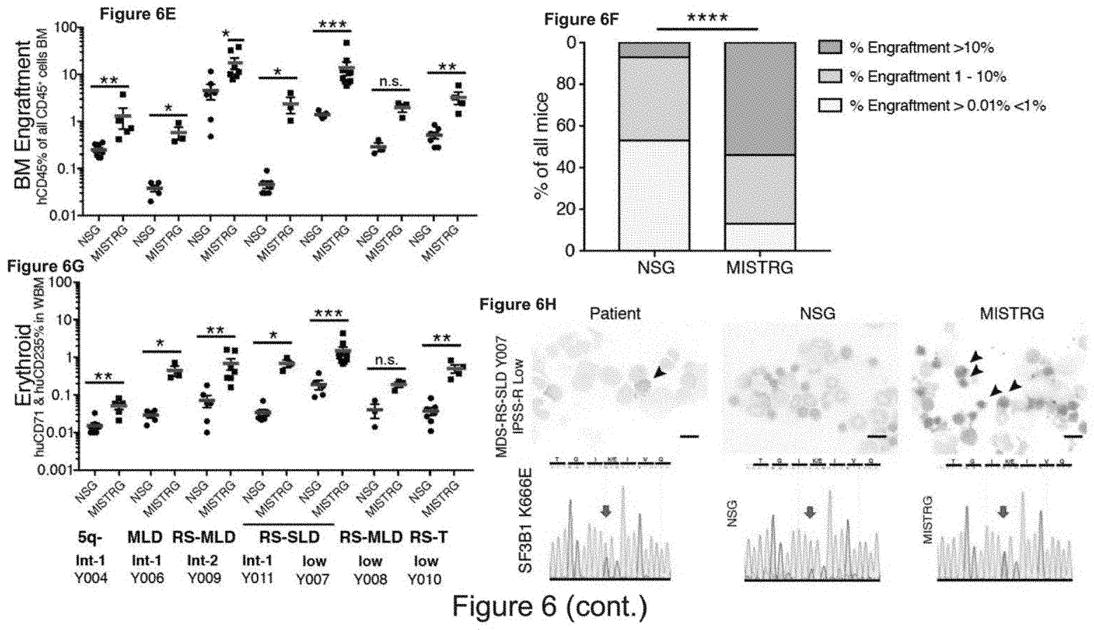

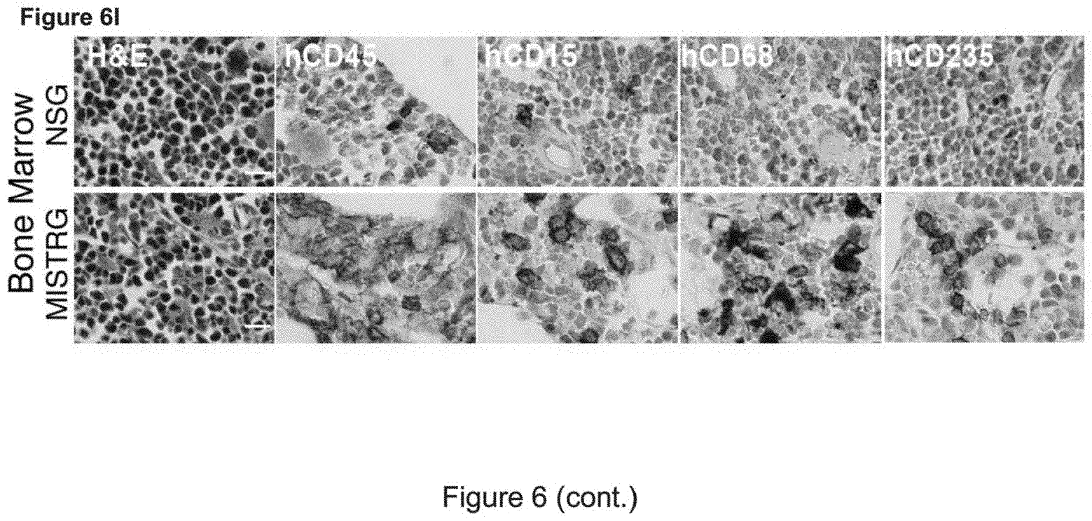

Primary HSC have a limited life-span in vitro, invariably lose their sternness over time, and normal IBC lines do not exist, in vitro differentiation assay's, such as colony forming unit assays and liquid culture, and leukemia cell lines serve as surrogates for some but not all processes. Bone marrow (BM), mobilized peripheral blood stem cells (PBSC), umbilical cord blood (UCB), and fetal liver (FL) are among the preferred sources for CD34+HSPCs. Fetal liver is richest for huCD34+while UCB and adult BM or PBSCs are a more accessible source. Efficiency of engraftment in immunodeficient mice is dependent upon the proliferative capacity of the HSC and decreases with age in the order of FL>UCB>adult HSPCs (Lepus C M et al., 2009, Hum Immunol., 70:790-802; Holyoake T L et al., 1999, Exp Hernatol., 27:1418-1427). Engraftment is particularly challenging for adult HSCs with almost 100-fold difference between FL and adult CD34 LISPCs. For the study of SCI), the stem cell source matters as FL- and IJCB-derived red cells almost exclusively express γ-globin. MISTRG mice were also shown to afford significantly higher overall engraftment of adult HSPC compared to NSG mice (Saito Y et al, 2016, Blood, 128:1829-1833; Song Y et al., 2019, Nature Communications). Importantly, MISTRG mice afford studies of normal and defective adult erythropoiesis with significantly higher BM erythropoiesis (FIG. 6) and the presence of ring-sideroblasts and dysplastic red cell precursors, in myelodysplasia (Song Y et al., 2019, Nature Communications) but absence of mature huRBC in PB.

Given the limited number of CD34+ cells in clinical specimens and the limited engraftment potential of adult HSPCs, efforts were focused to further improve engraftment in MISTRG mice. For this purpose, using CRISPR/Car9, the c-kit “W41” (V831M) mutation were introduced into fertilized eggs of the MISTRG strain. This mutation was originally identified within the “White-spotting (W) locus” responsible for abnormal pigmentation, blood formation, and gametogenesis (Geissler E N et al., 1981, Genetics, 97:337-361). Homozygous c-kifW41/W41 mutation results in mild (mouse) anemia without affecting fertility (Reith AD et aL, 1990, Genes Dev., 4:390-400; Nocka K et al., 1990, Embo J., 9:1805-1813).

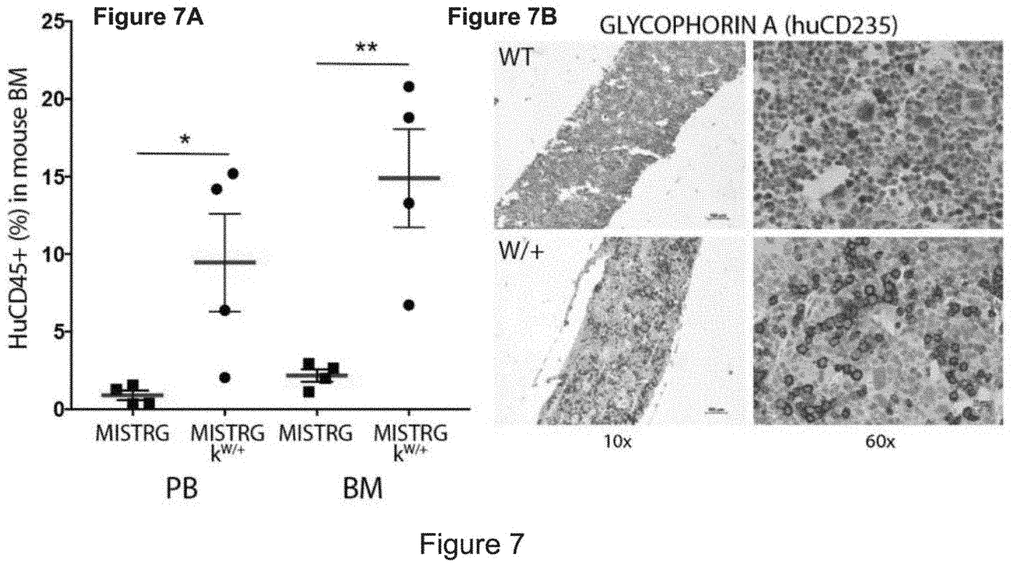

More recently, it has become clear that c-kit mutant HSC show reduced retention within the HSC niche, allowing engraftment of competing wild-type murine (Waskow C et al., 2009, Nature Methods, 6:267-269) and human (Cosgun KN et al., 2014, Cell Stem Cell, 15:227-238) HSC without irradiation. Similarly, the “erythropoietic niche” becomes receptive to human erythroid progenitors with robust establishment of UCB-derived erythropoiesis (Yurino A et al., 2016, Stem Cell Reports, 7:425-438; Rahmig S et al., 2016, Stem Cell Reports, 7:591-601). Thus, tests were conducted to assess whether adult HSPC could efficiently engraft c-kit W41 mutant MISTRG (MISTRGW41) mice without irradiation and whether the c-kit mutation would further improve erythropoiesis. Preliminary results of adult PSC engraftment in MISTRGW41het confirms efficient overall engraftment without irradiation (FIG. 7A) and robust erythropoiesis (FIG. 78). Crosses to homozygosity have now been completed and engraftment studies in MISTRGW41hom are underway.

### “Humanizing” the Erythroblastic Niche

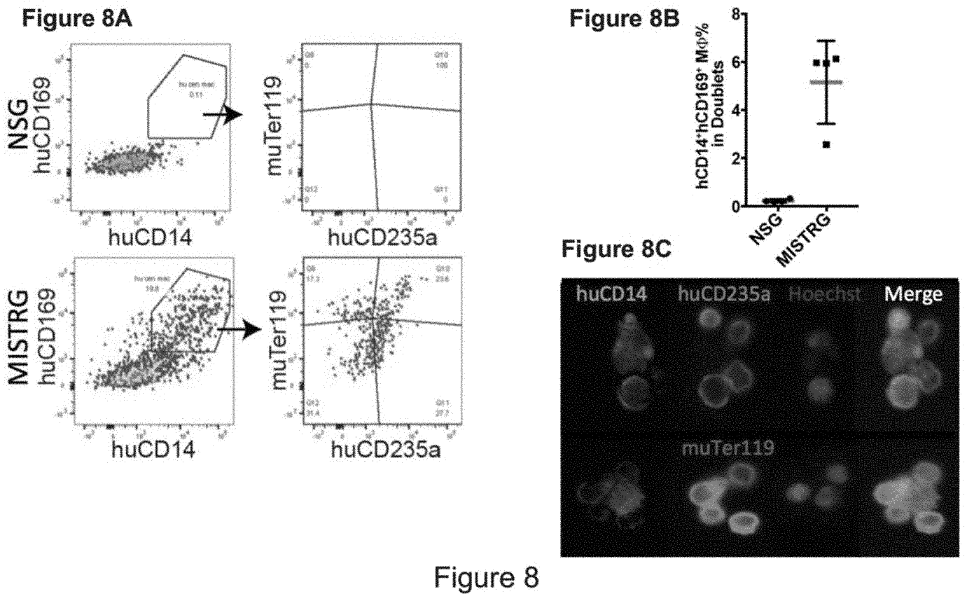

MISTRG mice express human M-CSF, a growth factor essential for terminal MO maturation that is poorly-cross-reactive between mouse and human. Unlike NSG, MISTRG mice efficiently repopulate host tissues with mature, functional resident tissue MΦs (Song Yet al., 2019, Nature Communications; Rongvaux A et aL, 2014, Nat Biotechnol., 32:364-372). Indeed, a subset of human MΦs in MISTRG mice are CD169+ and found within EBIs in close contact with erythroid progenitors of all differentiation stages (FIG. 8). Interestingly, human McDs are found in erythroblastic islands with human and murine RISC precursors.

This offers the opportunity to study the role of the central MΦ in human erythropoiesis and to dissect the roles of distinct MΦ in BM and spleen.

### Overcoming RBC (Immune) Destruction

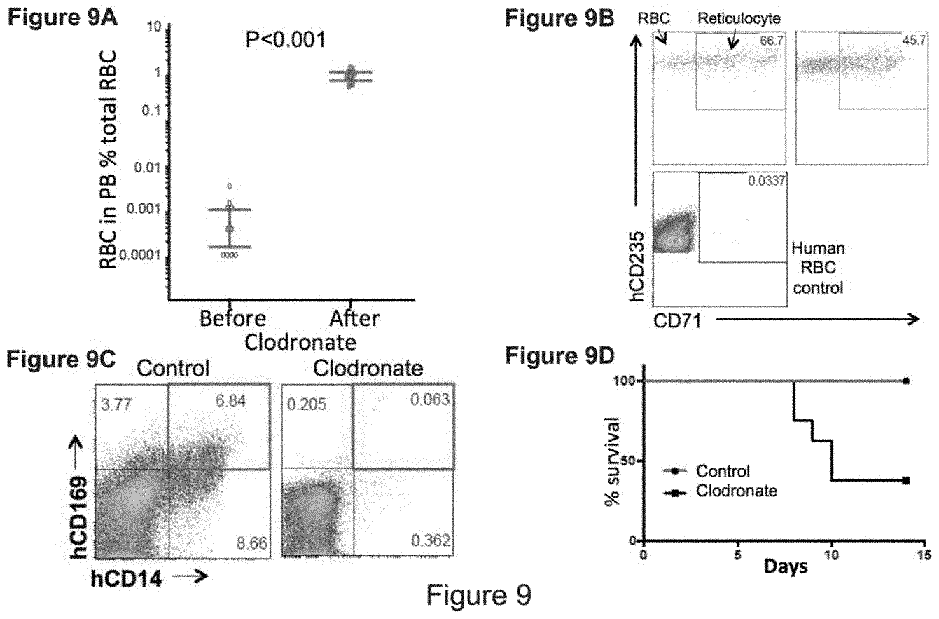

Despite improvements in the BM erythroblastic niche with enhanced erythropoietic engraftment and full maturation, mature RBC in PB are absent. As previously described for other models (Hu Z et al., 2011, Blood, 118:5938-5946), liposomal clodronate-mediated ablation of phagocytes transiently results in sparing of PB RBC in MISTRG mice, albeit in low numbers and with concurrent ablation of human CD169+ central MΦs with significant toxicity (FIG. 9). recent study explored potential MΦ-independent mechanisms of huRBC destruction and found that mouse complement may at least in part mediate the rejection of huRBC. huRBC survival is significantly prolonged when murine complement is eliminated by cobra venom factor (CVF) but only when MΦs are depleted concurrently with liposomal clodronate and with maximum human RBC chimerism at ˜3% (Chen B et at, 2017, Stem Cell Reports, 9:1034-1042).

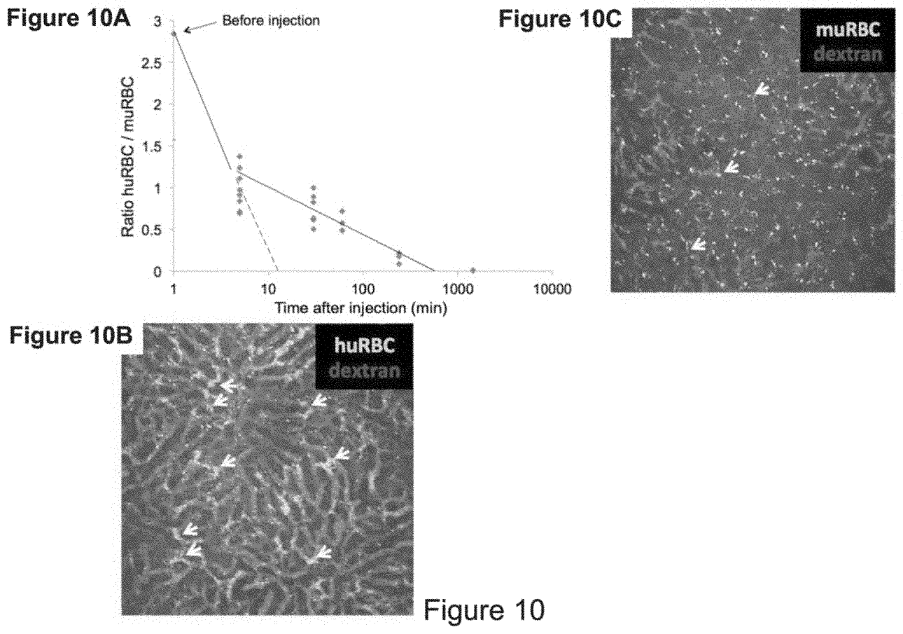

While the human spleen is thought of as the key organ sequestering and eliminating RBC tagged for destruction, Kupffer cells in the liver constitute 80-90% of the entire body's MΦs. Liver is also the site of complement synthesis. To determine the major site of huRBC destruction in the mouse, fluorescently labeled human or murine RBC were injected into MISTRG mice. Rapid preferential clearance of huRBC over muRBC (FIG. 10A) was confirmed and intra-vital imaging of spleen and liver was performed. HuRBC, but not muRBC, become rapidly and persistently trapped in the liver vasculature (FIG. 10B and FIG. 10C). Together, the present data suggests, that the mouse liver represents a major site of huRBC destruction.

### Human Liver Mouse

Although not bound by any particular theory, based on the data described above, it was hypothesized that replacement of the mouse liver with human liver may overcome destruction of mature huRBC in circulation. Deletion of the fumarylacetoacetate hydrolase gene (Fah^{-/-}) mimics the hereditary liver disease tyrosinemia type I and has been shown to allow selection of healthy murine (Overturf K et al., 1196, Nat Genet., 12:266-273) and human (Azuma H et al., 2007, Nature Biotechnology, 25:903-910) hepatocytes in vivo. Fah was deleted via CRISPR/Cas9 in the MISTRG background to generate MISTRGFah^{-/-} mice (in short MISTRGFah).

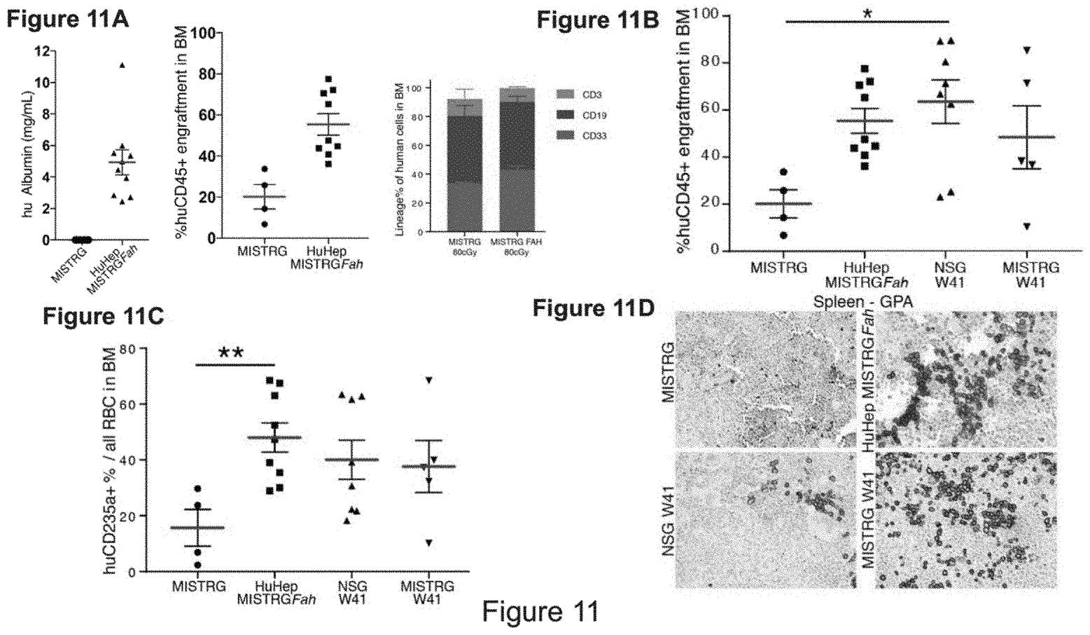

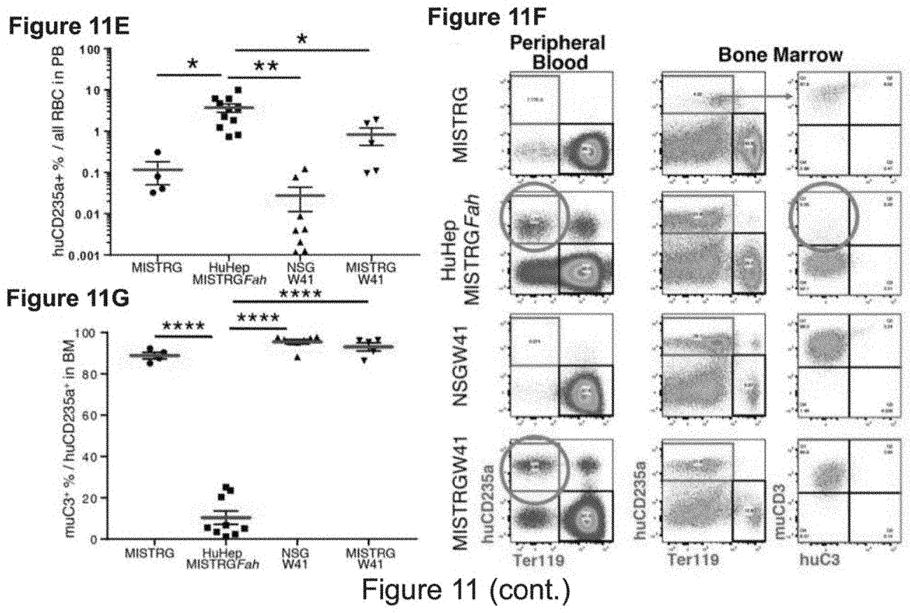

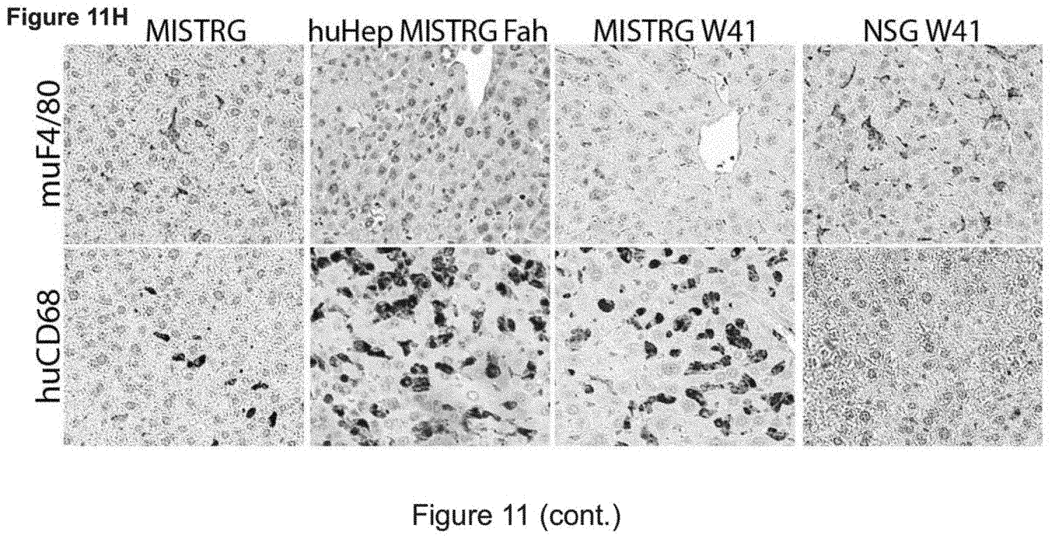

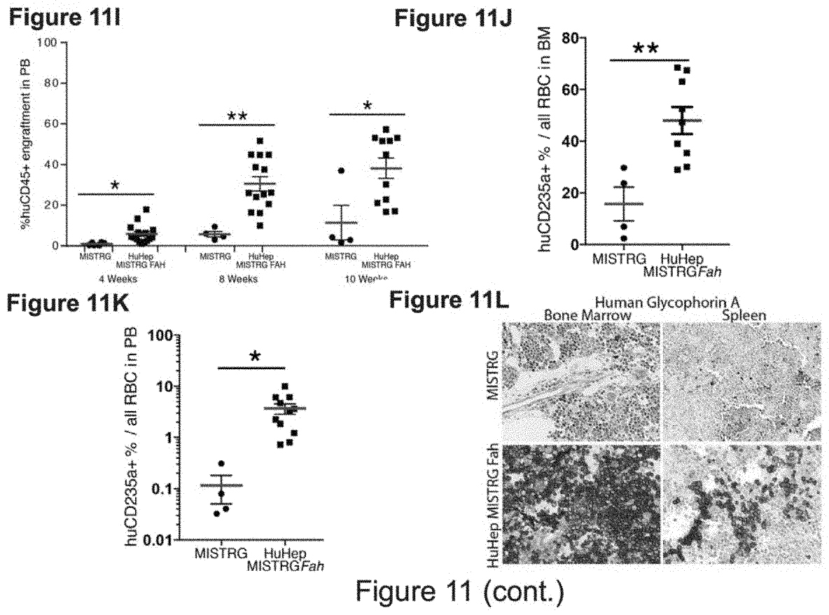

MISTRGFah are viable, fertile, and can be bred as homozygotes when maintained on drinking water supplemented with 2-(2-nitro-4-trifluoromethylbenzoyl)-1,3-cyclohexanedione (NTBC), that blocks tyrosine metabolism upstream of FAH and prevents buildup of hepatotoxic metabolites. At 8 weeks of age, MISTRGFah. mice were implanted with commercially available, adult human hepatocytes (HuHep) via direct injection into the splenic vein, followed by gradual withdrawal of NTBC water. Regulated buildup of intracellular FA results in death of murine Fah^{-/-} hepatocytes and regeneration with HuHep with up to 90% repopulation by HuHep (Azuma H et al., 2007, Nature Biotechnology, 25:903-910). When plasma human albumin levels reached 2 mg/dL (FIG. 11A), indicative of significant (˜80%) HuHep repopulation, HuHepMISTRGFah mice were sublethally (80cGy) irradiated and each mouse was engrafted with 105 fetal liver (FL) derived CD34+ cells. MISTRG (80cGy), MISTRGW41 and NSGW41 (no irrad) served as controls to assess the effects of human liver vs W41 mutation vs cytokine humanization on erythropoiesis and huRBC in circulation. 10 weeks post transplantation mice were analyzed for engraftment and. specifically erythroid maturation and PB huRBC.

All 4 strains were efficiently engrafted (FIG. 11B) with robust human erythropoiesis (huCD235a) in BM and interestingly in spleen with significant improvement over MISTRG (FIG. 11C and FIG. 11D). Importantly, only HuHepMISTRGFah (up to 10%) and MISTRGW41 (up to 1%) mice had circulating huRBC in PB, completely absent in NSGW41 and MISTRG mice (FIG. 11E), CD235a+ huRBC HuHepMISTRGFah and MISTRGW41 mice are enucleated (Hoechst neg) and mature as evident by loss of CD49d (ITGA4) and gain of Band3 staining (Hu J et al., 2013, Blood, 121:3246-3253). Interestingly, human erythroid cells in all strains but HuHepMISTRGFah mice are coated with murine complement C3 (muC3) (FIG. 11F and FIG. 11G) suggesting that liver humanization results in loss of murine C3 expression. Mu MΦs appear entirely replaced by human MΦs in HuHepMISTRGFah (FIG. 11H). These data suggest that huRBC destruction can be overcome by humanization of the mouse liver without compromise to the mouse longevity.

### SCD Models

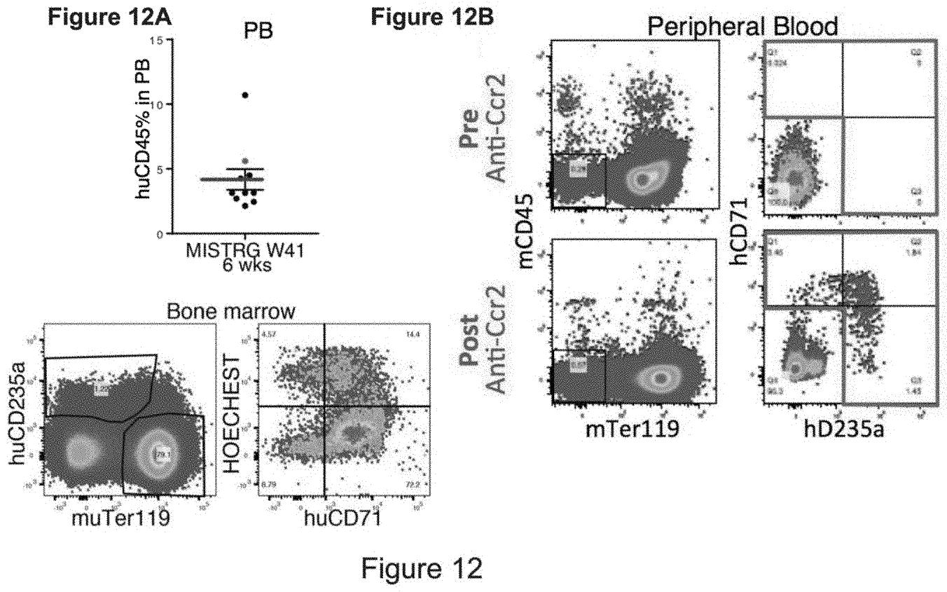

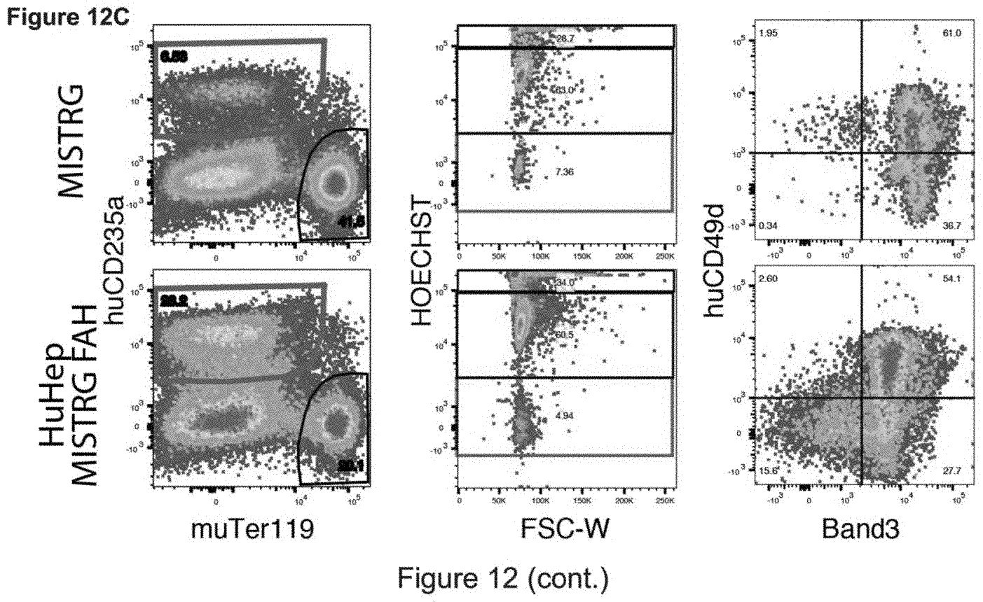

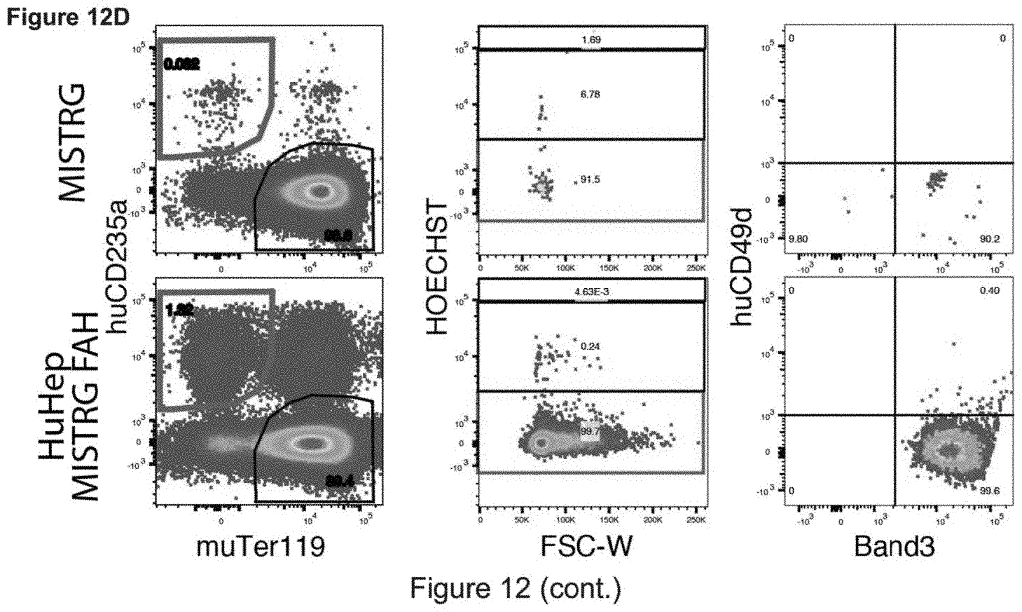

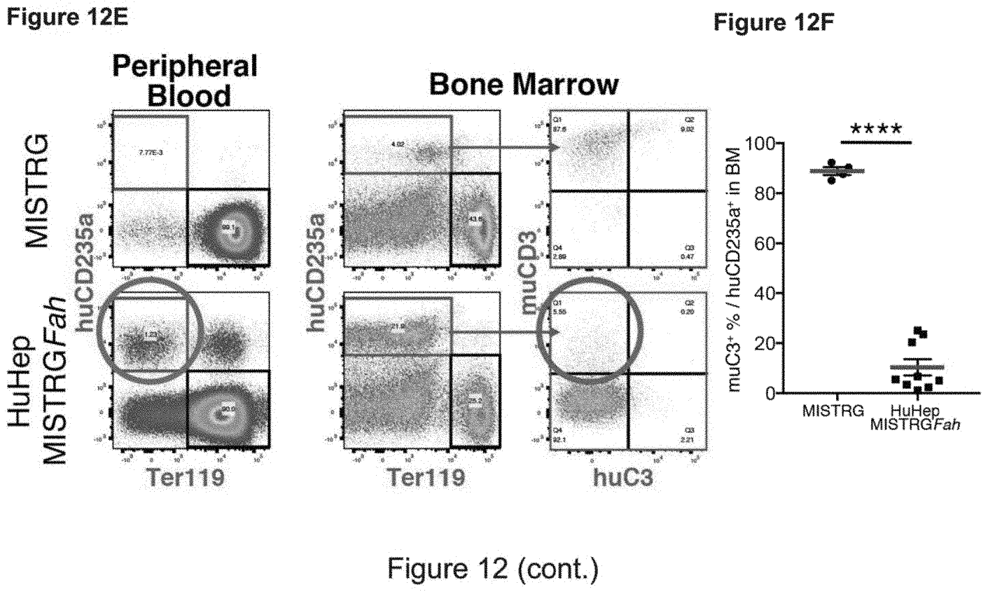

Encouraged by the results of both the 14IISTRGW41 and the HuHepMISTRGFah models, as a first step focused on testing engraftment of adult BM derived CD34+ cells from a patient with HgSS disease. N/IISTRGW41 mice were engrafted with 105 CD34+ cells. Preliminary engraftment determined at only 6 weeks post transplantation shows huCD45+ engraftment in PB and erythroid progenitors in BM in one select recipient mouse sacrificed for the purpose of this grant application (FIG. 12A). The present data confirms that adult human BM derived CD34+ from patients with SCD can engraft MISTRGW41 mice with establishment of human erythropoiesis.

Thus, described herein are strong preliminary data for a highly sophisticated in vivo model for the study of diseases of the human red cell and erythropoiesis in the context of human hematopoiesis and a human immune system. Preliminary evidence for a human liver—human cytokine mouse with highly efficient adult stem cell engraftment without need for toxic conditioning and with robust establishment of human, fully maturing erythropoiesis and circulating mature red cells is also provided. It is proposed to apply this model to the study of SCD, which is in dire need of novel therapeutic approaches and an in vivo model for the optimization of gene therapy approaches.

The data described herein demonstrate that the genetically modified non-human animals described herein represent a novel in vivo animal model of human hematopoiesis, as well as for human immune system.

The materials and methods employed in these experiments are now described.

### Experimental Strategy

The ultimate goal of this example was to develop the first ever in vivo model for primary human SCD to study the diverse SCD genotypes and phenotypes and for therapeutic testing. In the first two sections, the mechanisms that maximize erythropoiesis and protection of human RBC in circulation in the humanized murine host was determined. The mouse model was optimized to reliably achieve efficient engraftment and maturation of the erythropoietic lineage and seek to develop a reliable, easily transferable model with maximum contribution of huRBC to circulating RBC. In the third section, feasibility of modeling human SCI) and its systemic manifestations in vivo was assessed and the best model was applied to the study of potential treatments.

### Rigor and Reproducibility

Mouse sex/strains: Equal numbers of male and female mice were used and the effect of sex on experimental outcomes was tracked in analysis and reporting. The appropriate controls br experimental variables, such as several genotypes, vehicle for treatments, etc., were used and serial assessments were performed, as feasible.

Statistics: For all in vivo and in vitro experiments, sample sizes was chosen to assure adequate power to detect pre-specified effects at ≥0.05 significance level, using a 2-tailed t-test or one-way ANOVA as appropriate. Typically experiments were performed in 3 biological replicates with 5 or mice per condition/time point. Engraftment levels, phenotypic and genetic properties of human cells and their effects on recipient mice were the studied outcomes. All necessary tools and methods, including serial PB analysis, BM aspiration at key time points, and BM, PB, and tissue analysis were established via flow cytometry, Elisa, PCR, sequencing, histology, and ancillary molecular biology techniques to carefully evaluate the effects of variables under study.

For each primary sample, it was expected to be able to engraft 10-20 mice with an attrition rate of <10%. With significance at 0.05 and power of 80%, sample size of 5 mice per group enabled an effect size of 2.5 between treatment groups, For example, when the standard deviation of engraftment levels across mice in the same group was around 10%, a sample size of 5 in each group enabled a difference of 25% or higher between the groups. Similar biostatistical considerations applied across all sub-sections below and all estimates of sample sizes were evaluated in consultation with the YCCC biostatistics core. Controls, pitfalls, and alternative approaches are also detailed at the conclusion of each section.

### Timeline and Future Studies

The three sections were performed concurrently. The three sections address optimization of hematopoietic stem cell engraftment and erythropoiesis (Section 1), optimization of mature human RBC preservation in circulation (Section 2), and the modeling of SCD (Section 3). Ultimately all three sections converged in studies of SCD in the best humanized mouse developed in Section 1 and Section 2. These studies built the basis for pre-clinical studies in SCD, such as gene therapy and modulators of inflammation.

### Sections of Example 2

Established Methods and Techniques for Section 1 through Section 3

Engraftment of immunodeficient mice and extensive analyses and functional assays are routine in laboratories and were systematically applied to the questions below. All engraftments into MISTRG mice were performed as previously published (Deng K et al., 2015, Nature, 517:381-385; Song Y et al., 2019, Nature Communications), where MISh/hTRG were crossed to MiSm/mTRG to generate MISh/mTRG (in short MISTRG) recipient mice. All cells to be engrafted were pre-incubated with OKT3 antibody to deplete human T-cells (Song V et al., 2019, Nature Communications; Wunderlich M et al., 2014, Blood, 123:e134-144). Human fetal liver (FL), umbilical cord blood (UCB), and adult BM and peripheral blood mobilized stem cells (PBSC) were routinely used as human HSPC engraftment source.

For the proposed studies, adult HSPC were used whenever feasible as the ultimate goal was to generate a model of SCD where expression of adult HgS was key. Experimental and control mice were simultaneously engrafted with split donor grafts to minimize variability. 3 separate engraftment routes were routinely employed in the group depending on the questions asked and specific experimental circumstances: (i) intrahepatic injection into 1-3D old pups (Traggiai E et al., 2004, Science, 304:104-107), (ii) intrafetnoral, and (iii) retro-orbital engraftment into adult mice as performed for the data presented herein. Bleeds were routinely performed via tail vein or retro-orbitally to serially monitor engraftment and plasma proteins (such as cytokines, albumin, etc). Bone marrow aspirations were also routinely performed to assess bone marrow engraftment when PB analysis was not be reflective of BM status, such as the lack of huRBC despite robust erythropoiesis in the BM. Drugs and treatments, such as antibodies, small molecules, or cytokines via i.v., i.p., oral gavage, or nebulization routes were also routinely administered. For most engraftment experiments, mice were maintained for at least 10-12 weeks (and >16 weeks for assessment of HSC engraftment).

When the endpoint was reached, extensive flow cytometric analysis (EISC, lineage representation, myeloid/erythroid differentiation, RBC maturation and enucleation, reticulocyte stain (thiazole orange), and others); DNA sequencing and RNA expression analysis; PB and BM aspirate smear and histology of BM, spleen, liver, lung, kidneys were performed. PB, EM aspirate smears and cytospins of sorted populations were performed routinely. Multispectral imaging flow cytometry (IFC) of the erythroblastic islands in fresh BM from engrafted MISTRG mice was recently established by staining for the respective central macrophages (CD14, CD169) and erythroblasts (GPA, Ter119) in the “doublet population” in absence of any EDTA using the Amnis ImageStream imaging flow cytometer (FIG. 8 and Seu KG et al., 2017, Frontiers in Immunology, 8:1140). To assure specificity of the in vivo association of macrophages with erythroblasts, fresh BM from engrafted MISTRG mice (hu+mu) was analyzed concurrently with primary human and murine bone marrow alone, and an admixture of human and murine marrow after collection. Erythroblastic islands were uniquely formed in vivo and not an artifact generated by non-specific adhesion in the test tube.

Section 1: Optimize Erythropoiesis and Overcome Mechanisms of huRBC Destruction.

The most commonly available immunodeficient mouse models suboptimally support adult HSPC-derived erythropoiesis (EP) and lack mature human RBC in circulation. The above described data suggested that introduction of the murine c-kit “W41” mutation into the cytokine humanized MISTRG model provides significantly improved engraftment of adult HSPCs and robust human BM erythropoiesis (FIG. 7). As such, full characterization of the erythroblastic niche in MISTRGW41 mice and determination of whether addition of human erythropoietin (EPO) further improves EP maturation (Section 1a) is necessary. Previous publications and the above-described data implicate the mouse innate immune system and the liver as the site of huRBC sequestration and destruction, As such, determination of the mechanism and cellular component(s) that mediate(s) huRBC destruction and testing of the effect of mouse specific phagocyte depletion on huRBC survival were also performed (Section 1b).

### Section 1a: Optimization of the Erythroblastic Niche

MISTRG W41

C-kit W41 mutant MI(S)TRG mice were crossed to homozygosity for all knock-ins. Healthy adult BM CD34+ were engrafted and detailed flow-cytometric, histologic, and functional analyses were performed on PB, BM, and tissues >12 weeks after engraftment as published (Rongvaux A et at., 2013, Annu Rev Immunol., 31:635-674; Song Y et al., 2019, Nature Communications) and shown in the above data. MISTRG and NSGW41 mice served as controls for MISTRGW41 mice.

Erythropoietin

Erythropoietin is essential in in vitro differentiation of erythroblasts. While human EPO is partially cross-reactive towards murine cells, the reverse is not as clear. Studies by others suggest that EPO may improve EP in liposomal clodronate treated mice (when co-injected. with huIL3) (Hu Z et al., 2011, Blood, 118:5938-5946), but this has not been tested in mice with central human BM MΦs. Of note, MISTRG are already humanized for IL-3. huEPO (30U/mouse) or PBS were i.p. injected into engrafted MISTRGW41 and control mice and EP and %, number, and maturation of huRBC were assessed in BM (via BM aspiration) and PB before and after huEPO or vehicle administration.

Ervthropoietic Niche and Erythroblastic Islands

MISTRG mice are unique in that they express huM-CSF in physiologic manner from the endogenous murine locus and develop functional human tissue MΦs. The above-described data suggested that MISTRG BM and spleen huMΦs are CD169+ and form erythroblastic islands with human and murine erythroid progenitors. It is unknown whether human and murine central MΦs support erythropoiesis across species and whether huMΦs have an effect on huEP in transplanted mice. The relationship between MΦs and erythroid progenitors in MISTRGW41 (hu and muMΦ) vs NSG41 (only muMΦ) was elucidated via to specifically characterize the presence and distribution. of hu/hu, hu/mu, mu/hu, and mu/mu MΦ/erythroblasts islands. The degree of maturation and percentage of enucleated red cells (CD49d, Band3, Hoechst staining), expression of cell surface markers, such as VCAM on MΦs, and association with other cells (such as neutrophils) was determined (Seu K G et al., 2017, Frontiers in Immunology, 8:1140). The present model provides the unique opportunity to further characterize the central MΦ and its role in the erythroblastic niche and its function in erythropoietic differentiation and maturation in BM and spleen. Of note, the W41 mutation appears to open an erythroblastic niche also in the spleen, the site of stress erythropoiesis in mice, but not humans, which has not been described to date. Hu/hu, hu/mu, mu/hu, and mu/mu EBIs from BM +/− spleen were flow sorted and the EBI populations were subjected to (i) single EBI or single cell cytokine profiling using a platform combining subnanoliter microchambers and a high-density antibody barcode array, developed in the laboratory of Dr. Rung Fan, Associate Professor, Yale Department of Biomedical Engineering (Chen JJ et al., 2016, Methods Mol Biol., 1465:243-254; Xue Q et al., 2015, Science Signaling, 8:ra59) and (ii) to single cell sequencing (immediately after dissociation into single cell suspensions) on the 10× genomics platform readily available through the Yale Center for Genomic Analysis.

### Section 1b: Determine the Site and Mechanism of RBC Destruction and Selectively Target Murine Phagocytes

Although not bound by any particular theory, it was hypothesized that identifying the site and mechanism of huRBC destruction enables development of the best possible model for the study of erythropoiesis and diseases of the human red cell.

The Site of huRBC Destruction

In humans the spleen is considered the major site of RBC destruction in auto-immune and other RBC disorders frequently resulting in significant improvement when splenectomy is performed. Splenectomy was performed in transfused and engrafted MISTRG mice without effect on huRBC survival. The intra-vital imaging suggested that huRBC get rapidly sequestered in the murine liver (FIG. 10) consistent with the fact that the liver contains 80-90% of tissue macrophages. These results were confirmed and quantitated in comparison to other organs, such as spleen. Intra-vital imaging were performed in CFSE labeled huRBC infused huCD34+ engrafted NSGW41 and MISTRUW41 to dissect the effect of murine vs human MΦs (FIG. 11H) on RBC sequestration. Reduced sequestration was expected in engrafted MISTRGW41 in which muMΦs are at least partially replaced by huMΦs. Alternatively, since so-called scavenger receptors, known to aid in phagocytosis of damaged RBC (Chulay J D et al., 1990. The American Journal of Tropical Medicine and Hygiene, 43:6-14; Terpstra Vet al., 2000, Blood, 95:2157-2163; Erwig L P et al., 2008, Cell Death and Differentiation, 15:243-250) are expressed not only on “professional phagocytes” but also on endothelial cells and hepatocytes and these may contribute to huRBC sequestration. Image flow cytometry were performed on liver hematopoietic, parenchymal, and endothelial isolated via collagenase perfusion and differential centrifugation (Cabral F et al., 2018, J Vis Exp., 132) to determine which cells contain intracellular CFSE labeled huRBC in the engrafted and non-engrafted NSGW41 and MISTRGW41 hosts.

Murine Macrophages

Human M-CSF remained cross-reactive to murine MΦs and MISTRG mice maintained MΦs from both species. Mice deleted for the M-Csf receptor gene Csf1r had. poor viability due to effects on other organs such as bones, teeth, and brain (Wiktor-Jedrzejczak W et al., 1991, Exp Hematol., 19:1049-1054; Dai X M et al., 2002, Blood, 99:111-120; Erblich B et al., 2011, PLoS One, 6:e26317). Csf1r deleted MISTRG mice were also generated and transfused fluorescently labeled RBC showing comparable survival of human to murine RBC. However, MISTRGCsfr1^{-/-} mice could not tolerate human CD34 engraftment with maximum post-engraftment lifespan of ˜4 weeks. Thus, alternative options were sought to specifically deplete murine tissue macrophages without the detrimental effects of clodronate.

Mack et al. have shown that Ccr2 is essential for macrophage egress from the BM to peripheral tissues and a murine-specific anti-Ccr2 antibody effectively reduces egress of murine BM macrophages (Mack M et al., 2001. The Journal of Immunology, 166:4697). This anti-Ccr2 antibody and isotype control, obtained as gifts from Dr. Mack, were injected engrafted MISTRG mice every other day x 5. The resulting data showed that erythroid maturation and % huRBC in circulation were significantly improved suggesting that blocking egress of macrophages to tissues may decrease huRBC destruction (FIG. 12B). These findings were also tested in NSGW41, MISTRG, and MISTRGW41 mice engrafted with adult CD34+ HSPC. PB huRBC contribution and persistence, BM erythropoiesis, and BM and tissue macrophage distribution were also assessed. The anti-Ccr2 antibody treatment were also tested in the liver humanized model in Section 2, Unlike Csfr1 KO mice, Ccr2 KO mice were viable and healthy. Thus, expanding these results should be likely to knock out Ccr2 in MISTRGW41 mice using CRISPR/Cas9 and cross mice to homozygosity on the most promising background.

Complement

Previous studies have shown that huRBC in the murine host were coated with murine C3 which may target huRBC to murine macrophages and other phagocytes (Chen B et al., 2017, Stern Cell Reports, 9:1034-1042). Administration of cobra venom factor (CVF) activated and thereby exhausted C3 and C5 complement and increased the % of circulating huRBC, but interestingly only in conjunction with liposomal clodronate in NOD/SCID mice (Chen B et al., 2017, Stem Cell Reports, 9:1034-1042). Of note, mice of the NOD background lacked complement C5 (Baxter A G et al., 1993, Diabetes, 42:1574-1578), while mice of the RG background were fully competent. The effect of CVF administration (5 μg per mouse d-1 and d0 before RBC transfusion) on survival of fluorescently labeled RBC injected into un-engrafted NSGW41 and MISTRGW41 and on PB huRBC survival in engrafted NSGW41 and MI5TRGW41 were determined. CYP was combined with anti-Ccr2 antibody treatment to determine whether there is synergy between complement depletion and muMΦ reduction, intriguing since C3 can also be synthesized by myeloid-derived cells at the site of action. If depletion of complement resulted in significant survival of huRBC in MISTRGW41 mice, knocking out C3 (Holers VM, 2000, Immunopharmacology, 49:125-131) in MISTRGW41 mice +/− knockout of Ccr2 were considered. Studies of complement contribution were also a part of the humanized liver studies in Section 2, as liver is the predominant site of synthesis of most complement factors.

### Section 1: Expected Results, Potential Difficulties and Alternative Approaches

Greatly enhanced erythropoiesis is expected in MISTRGW41, Administration of human EPO may enhance overall erythropoiesis and final maturation. :If the effect was significant, erythropoietin was humanized via knockin of the human gene into MISTRGW41 mice. Maturation of RBC is expected in all models subject to study (NSGW41, MISTRG, MISTRGW41) given published reports but to different degrees. Enhanced maturation and enucleation of human RBC in mice with functional huMΦs is also expected due to critical function of the MΦ in this process. It is expected that while murine MΦs are likely to phagocytose huRBC, huMΦs does not phagocytose human RBC but aid in their maturation. If successful, selective reduction of tissue muMΦs via anti-Ccr2 antibody treatment (Section 1b) should thus further enhance human erythropoiesis. Interplay between human and murine RBC and MΦs were tested in the imaging studies with and without depletion of complement with CVF (or humanization of the liver, see Section 2).

A confounding factor that was taken into consideration is the fact that huMΦs in MISTRG phagocytose murine RBC and thereby create anemia accompanied by mouse stress erythropoiesis in the spleen. The data suggested that lack of need for irradiation in c-kit mutant strains ameliorates phagocytosis of murine RBC likely due to decreased “immune-priming” due to reduced cell damage, inflammation, and antigen presentation. These parameters were evaluated with all appropriate controls. Human RBC (MCV 80-96 fl) are roughly twice the size of murine RBC (MCV 45-55fl). HuRBC could thus get trapped in murine capillaries due to their larger size. However, the RBC membrane elasticity allows RBC to pass through capillaries that are narrower than their own diameter. Imaging studies in “huRBC tolerant” mouse models in this proposal may give insights. Studies in Section 1 in conjunction with Section 2 maximize erythropoiesis and dissect the mechanisms of RBC destruction and guide development of future generations of genetically modified MISTRG mice.

### Section 2: Humanization of the MISTRG Liver to Generate a HuRedCell Mouse.

The liver is the site of production of numerous proteins essential for immune defense, such as complement, and the largest reservoir of tissue macrophages (Kupffer cells constitute 80-90% of the entire body's macrophages). Although not bound by any particular theory, given the results that demonstrate that infused huRBC are rapidly sequestered in the mouse liver, it was hypothesized that liver humanization may significantly improve huRBC survival. The liver was successfully humanized in MISTRGFah mice, followed by human IISPC engraftment, resulting in robust BM erythropoiesis and a significant percentage of huRBC in circulation (FIG. 11). Section 2 builds upon these promising preliminary data to understand how humanization of the liver spares huRI3C and to determine whether additional modifications based on the results in Section 1 can further increase huRBC % in the PB.

### Section 2a: Determine the Effect of Liver Humanization on RBC Survival and Hematopoiesis

RBC destruction

HuRBC are rapidly sequestered in the mouse liver as evident by flow cytometric analysis and intra-vital imaging. Liver humanized HuHep- and control .MISTRGFah mice were transfused with fluorescently labeled human and murine RBC as in Section 1 and (i) determine rate of clearance of hu vs muRBC, (ii) determine the site of clearance by quantitating hu vs muRBC sequestration in liver versus spleen, lung, BM, or kidney by flow-cytometry and intra-vital imaging of the liver. HuRBC was expected not be retained in the liver of HuHepMISTRGFah mice, similarly to muRBC in control mice. If significant trapping of huRBC in the humanized liver continued, image flow cytometry was repeated on hematopoietic and non-hematopoietic liver cells (as in Section 1b) to determine which cell type is responsible for huRBC trapping and clearance within the humanized liver.

Macrophage Populations

MISTRG mice engrafted with human CD34+HSPC populate non-hematopoietic tissues with human tissue macrophages (Song Y et al., 2019, Nature Communications; Rongvaux A et al., 2014, Nat Biotechnol., 32:364-372). However, murine macrophages co-exist in significant numbers (FIG. 11H). The general view is that in the homeostatic situation macrophage populations are maintained by local proliferation. In pathological conditions, such as inflammation, macrophages may be replaced by circulating monocytes that differentiate into tissue resident macrophages (Klein I et al., 20067, Blood, 110:4077-4085; Scott CL et al., 2016, Nature Communications, 7:10321). Replacement of murine with human hepatocytes may accelerate circulating human monocyte recruitment and differentiation into liver tissue macrophages. Tests were conducted to determine whether humanization of the liver alters human versus murine macrophage ratios and phenotypes in huCD34+ HSPC engrafted Huliep- compared to control MISTRGFah mice via flow-cytometry and histology.

Complement

The liver is the major site of complement expression. The present results suggested that HuHep-MISTRGFah mice lack murine complement C3 evident by lack of human erythroid cell coating with muC3 (FIG. 11F and FIG. 11G) and presence of small amounts of huC3 on muRBC. Experiments were also conducted to determine expression of murine versus human complement factors (C1, C2, C3, C5, CFB and others) in HuHep- and control MISTRGFah mice by Q-RT-PCR and Elisa. HuRBC with native and heat-inactivated serum (complement factors are heat labile) from HuHep and control MISTRGFah mice were pre-incubated., and pre-incubated fluorescently labeled huRBC was transfused into HuHepMISTRGFah and control mice to determine the contribution of complement to huRBC destruction. Prolonged survival of huRBC infused into HuHepMISTRGFah mice were expected when pre-incubated with HuHep or heat inactivated, but not control MISTRGFah serum. The effects of serum pre-incubation should not differ when huRBC are infused into control mice without liver humanization.

Section 2b: Optimize Erythropoiesis and huRBC Survival in HuHepMISTRGFah Mice

The obtained data suggested feasibility of humanization of the mouse liver in MISTRGFah mice followed by engraftment of human CD34+ HSPC resulting in significant increase in human RBC in circulation. Thus, this section focused on establishing as well as further improving this valuable model.

Humanization of Liver

The procedures to assure maximum liver humanization in the shortest possible time and highest possible engraftment of human CD34+ cells while preserving maximum viability of mice was optimized. Human hepatocytes are readily available from commercial vendors (4-8×106/vial from Thermofisher). Importantly, a previous study suggested that HLA matching of hematopoietic cells and hepatocytes is not necessary (Wilson E M et al., Stem Cell Research, 13:404-412) allowing engraftment with a panel of CD34+ cells with the same liver.

Hepatocyte Injection and N717.13C Withdrawal

The data suggested that liver humanization did not limit survival of MISTRG mice with >80% viability in the preliminary experiments. Best viability was achieved when hepatocyte injection was performed at ≥8 weeks of age. Experiments are conducted to confirm the preliminary data showing that maximum liver humanization was achieved by ˜90 days after hepatocyte injection and that human albumin concentration >2mg/dL correlated with >80% liver humanization. NTBC water withdrawal is followed according to Azuma et al. (Azuma FL et al., 2007, Nature Biotechnology, 25:903-910).

C-kit W41 Mutation and Erythropoietin

The data in Section 1 suggested that the c-kit W41 mutation significantly enhanced overall and erythropoietic engraftment. MISTRGW41 X MISTRGFah mice were crossed to obtain MISTRG-ckitW41/W41⁻Fah^{-/-}, in short MISTRGW41Fah mice. Human hepatocyte transplantation was followed by huCD34+ engraftment and assured liver humanization and efficient engraftment compared to MISTRGW41 and MISTRGFah mice by the standard assays. A recent report found that 10% of EPO was made in the liver (de Seigneux S et al., 2016, Journal of the American Society of Nephrology, 27:2265-2269). Analysis was conducted to determine whether transplanted human hepatocytes expressed huEPO via Q-RT-PCR and Elisa. While low-grade irradiation. was non-toxic to human hepatocytes, lack of irradiation was expected to be advantageous on overall survival of HuHep mice.

Ervthroblastic Islands

Analysis was conducted to confirm the findings regarding the erythroblastic niche as in Section 1 and to determine whether liver humanization altered composition of EBIs in BM or spleen. Of note, not only PB RBC but also BM erythropoiesis appeared improved in HuHepMISTRGFah compared to MISTRG mice (FIG. 11). Erythropoiesis in HuHepMISTRGW41Fah mice were characterized in comparison to HuHepMISTRGFah and MISTRGW41Fah mice. Understanding the mechanism may shed light on fundamental basic mechanisms of anemia associated with liver disease.

### Section 2 Results, Potential Difficulties and Alternative Approaches

These data proved feasibility to generate liver and cytokine humanized mice with robust erythropoiesis and circulating RBC. This section seeks to understand how humanization of the liver achieved these exciting results that were observed and to further improve upon these findings. By incorporation of the findings from Section 1 into the present model with liver humanization, experiments focused on development of a robust model for adult BM derived hematopoiesis and erythropoiesis. At the same time, studies may eventually allow to generate a robust model without requirement of liver humanization, which, while feasible and performed in several labs, may not be universally transferable. Combination of EPO knockin with murine complement factor (e.g., C3) and Ccr2 knockout in the MISTRGW41 background may be such a constellation.

The ultimate goal was to generate mice with predominance of human RBC in circulation, but for the purpose of SCD (Section 3) studies, at least >30% (based on clinical observations and guidelines in SCD to maintain HgS <30% in populations at risk or presenting with acute complications) was desired. Lower huRBC% may be sufficient for disease manifestation since human RBCs are ˜twice the size of murine RBC, potentially resulting in slowed passage through capillaries and reduced oxygenation of tissues. The in vivo imaging studies should provide the necessary insights. Measurements of hu vs muRBC flow velocity via intravital imaging of other vessels in the mouse, such as on the ventral aspect of the ear dermis, are likely to enable the determination of velocity in relation to vessel diameter (Honkura N et al., 2018, Nature Communications, 9:2746). Such intravital imaging is fully established by the Yale In Vivo Imaging Core where previous in vivo imaging of hu vs muRI3C in liver and other organs was performed. The core provides imaging by core staff as well as training. For the purpose of the study of SCD (Section 3) slowed flow may be beneficial to elicit disease manifestations even at lower human sickle RBC % due to longer dwell time in deoxygenized tissues,

### Section 3: Model SCD in the HuRedCell Mouse

Several murine sickle cell models exist, each with unique shortcomings. In general, they represent complex genetic models that cannot be flexibly altered to study primary patient variants. Globins are mostly expressed from a single allele with concurrent deletion of the mouse alleles in mouse RBC with low MCH and MCHC (Nagel RL et al., 2011, Br J Haematol., 112:19-25; Parker M P et al., 2018, Methods Mol Biol., 1698:37-65). An in vivo model of primary human SCD would represent a major step forward in the ability to study disease heterogeneity and test patient centered therapeutics. Modeling primary human SCI) requires engraftment of post-natal (pediatric and adult) samples that predominantly express 13-globin, as FL- and UCB-derived HSPC generate RBC with predominant γ-globin expression. Although not bound by any particular theory, it was hypothesized that the present MISTRGW41 based model is ideally suited to engraft SCD-derived HSCP with development of robust erythropoiesis. Successful liver humanization and additional modifications proposed in Section 1 and Section 2 are likely to result in significant contribution of huRBC to circulating RBC with high likelihood that can be used to study primary SCD in vivo.

CD34+ HSPC from SCD patients (>6 mths and preferentially adult due to availability) were engrafted into liver-humanized HuHepMISTRGW41Fah mice and characterize overall engraftment, erythropoiesis, RBC parameters, β-vs γ-globin expression, RBC sickling in tissues and PB, and systemic SCD manifestations, compared to aged matched healthy control HSPC-engrafted mice. Effects of huSickle RBC blood counts, mouse survival, and end-organ damage during steady state and after induction of VOC were determined.

Optimize SCD CD34+ Engraftinent and Establish SCD Specific Analysis

The obtained data suggested that SCD derived CD34+H SPC engraft in MISTRGW41 mice and give rise to BM erythropoiesis (FIG. 12) but negligible PB huRBC as expected. Several BM samples with sufficient cell numbers are stored in the Hematology Tissue Bank and biobanking efforts are ongoing. CD34+ cell number in HuHepMISTRGW41Fah mice are being optimized to maximize BM engraftment and establishment of erythropoiesis while maximizing the number of mice engrafted. HuHepMISTRGW41Fah mice were engrafted with SC (HuSickleMouse) vs control (HuRedCellMouse) CD34+ HSPC and serially monitored mice via tail bleeds for overall and lineage engraftment and PB huRBC using flow-cytometry, PB smear for presence of sickled RBC, CBC, and hemoglobin electrophoresis in cellulose acetate (in alkaline conditions, with appropriate human and marine controls, routine in the Yale clinical laboratory). Depending on the results from Section 1 and Section 2, erythropoietin, anti-Ccr2 antibody, complement inhibitor were administered or next generation MISTRGW41-derived mice generated during this grant period to maximize erythropoiesis and PB huRBC was chosen.

Demonstrate SCD Pathgenesis in SC Engrafted HuHepMISTRGWL41Fah Mice

The present data strongly suggested a successful generation of a model of human primary SCD with systemic manifestations of sickle cell disease. Although not bound by any particular theory, it was hypothesized that these HuSickleMice should have systemic effects depending on the final percentage of huRBC in circulation, HgS vs HgF distribution, degree of anemia. Hemolysis labs (CBC, LDH, bilirubin, haptoglobin), urine concentrating ability, and overall survival in SC vs Ctrl-engrafted mice were measured and results were correlated closely to % huRBC and degree of anemia. Careful analysis of tissues at the analysis endpoint for signs of vaso-occlusion, such as tissue scarring, was also conducted.

Induction of Vasoocclusive Crisis (VOC)

Infection and inflammation are major triggers of vaso-occlusive crises in SCD. It is possible that the present SCD engrafted HuHepMISTRGW41 Fah mice are protected from VOC at baseline due to low (<30%) contribution of huRBC to total RBC. Administration of lipopolysaccharide (LPS) in low doses has been shown to effectively induce lung vasoocclusion in SCD mice (Bennewitz MR et al., 2017, JCI Insight, 2:e89761), LPS (i.v. or i.p. injection or nebulization) was successfully administered to engrafted MiSTRG mice and monitored tissue and lung pathology and immune cell infiltration. LPS should be administered to SCD and control engrafted HuHepMISTRGW41Fah mice via i.p. injection or nebulization and monitor mice for general signs of VOC, such as altered behavior (failure to groom, hunched posture, ruffled fur, weight loss), and parameters of hemolysis and organ damage can be determined. RBC flow via intra-vital imaging can be measured as performed in Section 1 and Section 2. For nebulized mice, effects of inflammation on sickling in the lung via intravital imaging can be determined, and terminal analysis can be conducted with bronchoalveolar lavage and lung histology and fluorescent immunohistochemistry of frozen sections for vaso-occlusion and lung inflammatory infiltrates.

### Section 3 Expected Results, Potential Difficulties and Alternative Approaches

HuHepMISTRGW41Fah mice engrafted with SCD huCD34+HSPC were expected to develop SCD. The level of human erythropoiesis in the bone marrow, the output of huREC into circulation, and the degree to which huRI3C get destroyed either intravascularly due to hemolysis (expected to be due to RBC sickle manifestations) or extravascularly (due to SC and immune-mediated destruction) should influence SCD manifestations. Detailed measurements of these parameters should be taken into consideration in all interpretations. Patients with SCD transfused or exchanged to HgS<30% are clinically protected from vaso-occlusion. if unable to raise huRBC % to >30%, manifestations of SCD may not be detectable. However, huRBC are twice the size of muRBC and their passage through deoxygenated tissues may be sufficiently slowed to result in disease manifestations at lower huRBC % with endorgan damage reflective of damage in SCD patients, such as dilated, packed vessels resulting in scarring infarcts.

SCD manifestations are not solely dependent on huRBC sickling but on interactions with other cells. Humanized mice have murine and human myeloid cells and murine and human platelets both of which are expected to be affected by the inflammatory changes encountered in SCD. SCD patients become functionally asplenic and eventually “auto-splenectomized”. Splenic histology, human engraftment, and the human immune system in SCD can be analyzed vs control engrafted mice.

One of the major advances of the present HuHepMISTRGW14Fah model is the fact that huRBC survive in circulation. If it is not possible to develop a stable model of SCD, the option of transfusing unlabeled or CFSE labeled normal should be explored vs sickle huRBC into unengrafted HuHepMISTRGFah mice, and life-span of normal vs sickle huRBC (as in Section 2) and the ability to raise huRBC levels to >30% with repeat transfusions over time can be determined. In the worst case, the present HuSickleMice do not develop disease manifestation of SCD due to insufficient huRBC % and/or absence of significant sickling in circulation and tissues. In that case, the model is still highly to be beneficial for the study of SCD gene therapy or other therapeutics modifying hemoglobin balance (β^{s}vs γ) as gene corrected cells and clonal composition can be traced in vivo, in the future, the present model could also be ideally suited for the study of infections of the RBC, such as malaria (in normal or SCD engrafted mice), where the humanized liver would serve to propagate infectious particles in vivo (Vaughan AM et al., 2012, Future Microbiology, 7:657-665).

In summary, the present studies result in a unique model of primary human RBC disorders and specifically SCD and promise to significantly aid in study of disease mechanism and contribute to novel therapies and pre-clinical studies.

### Example 3

### Development of a Mouse Model for Human Blood Stage Malaria Infection Humanized Mouse Models for Plasmodium Blood Stage Infection

Plasmodium species infect human reticulocytes and erythrocytes. Transfer of mature RBCs has enabled to model blood stage infection with P. falciparum (Jimenez et al, 2009). Current state-of-the-art mouse models do not support sustained human erythropoiesis, Thus, although not bound by any particular theory, it was hypothesized that the microenvironment in the mouse fails to support erythropoiesis.

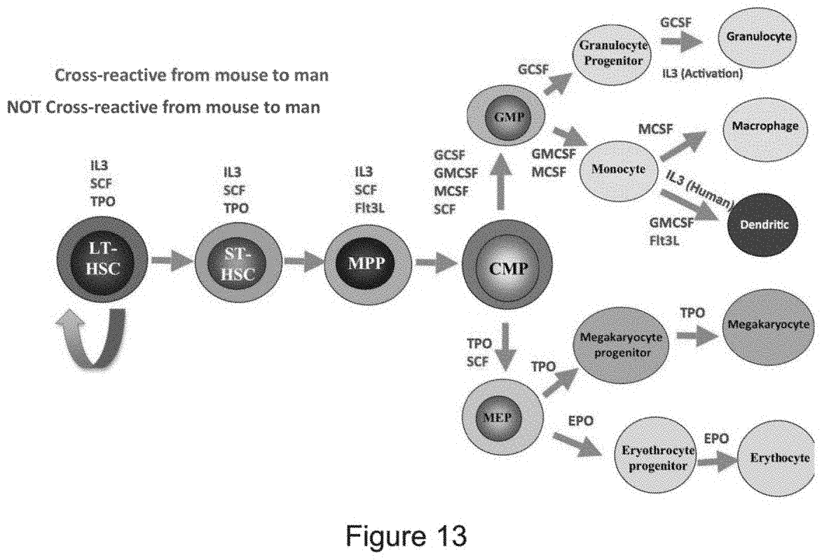

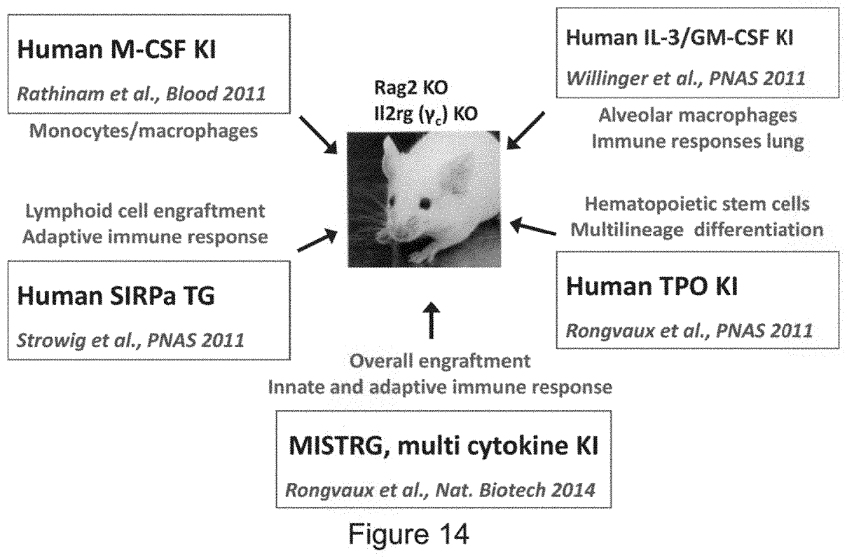

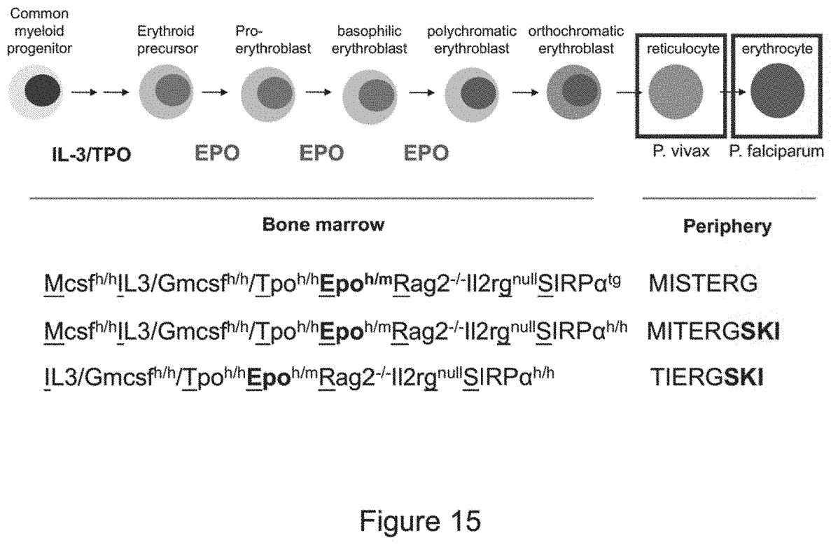

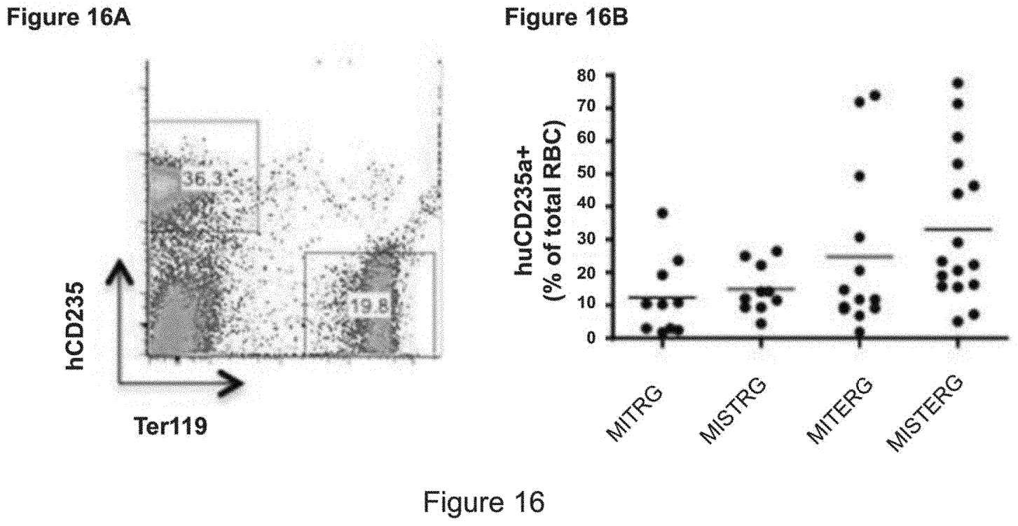

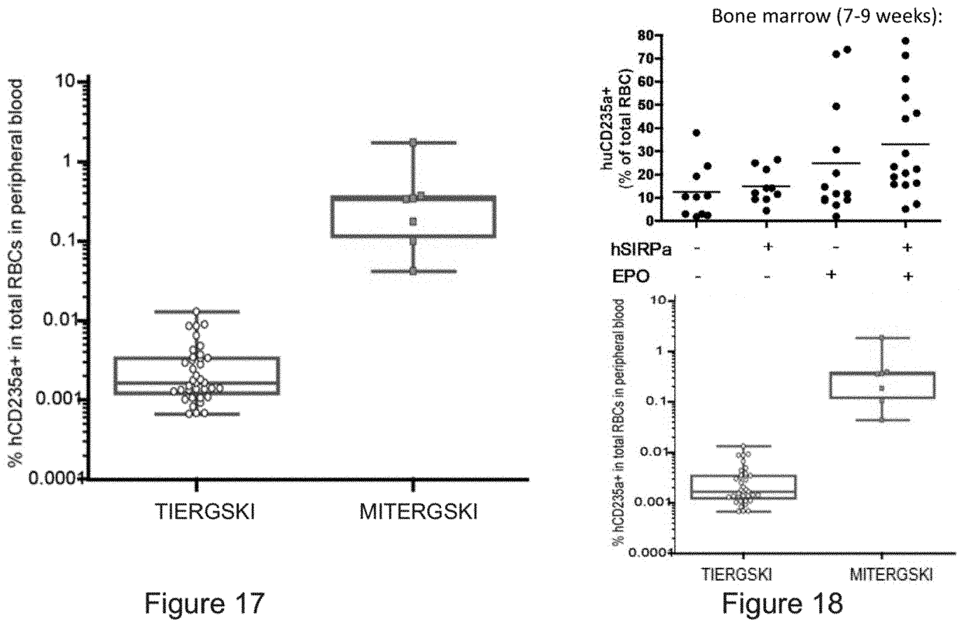

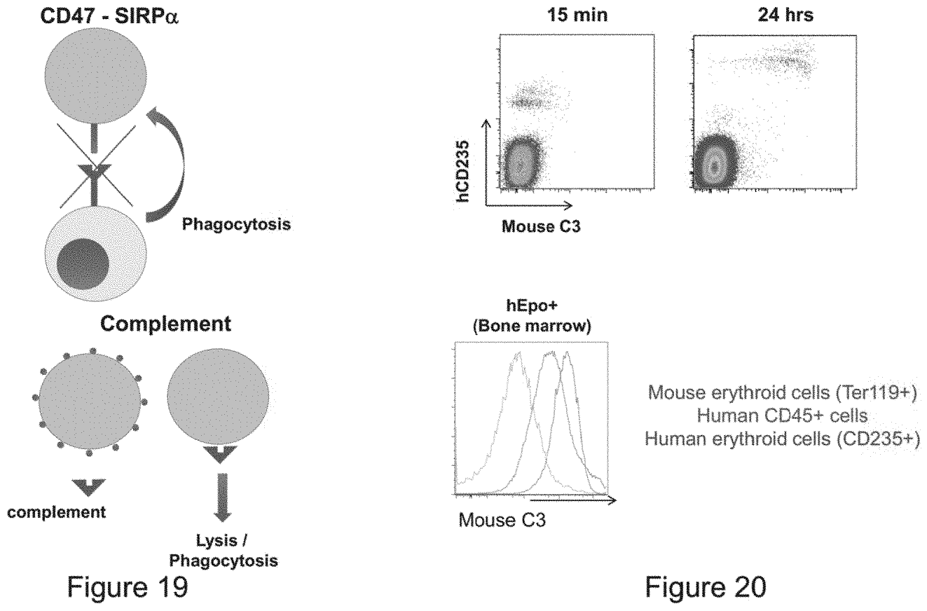

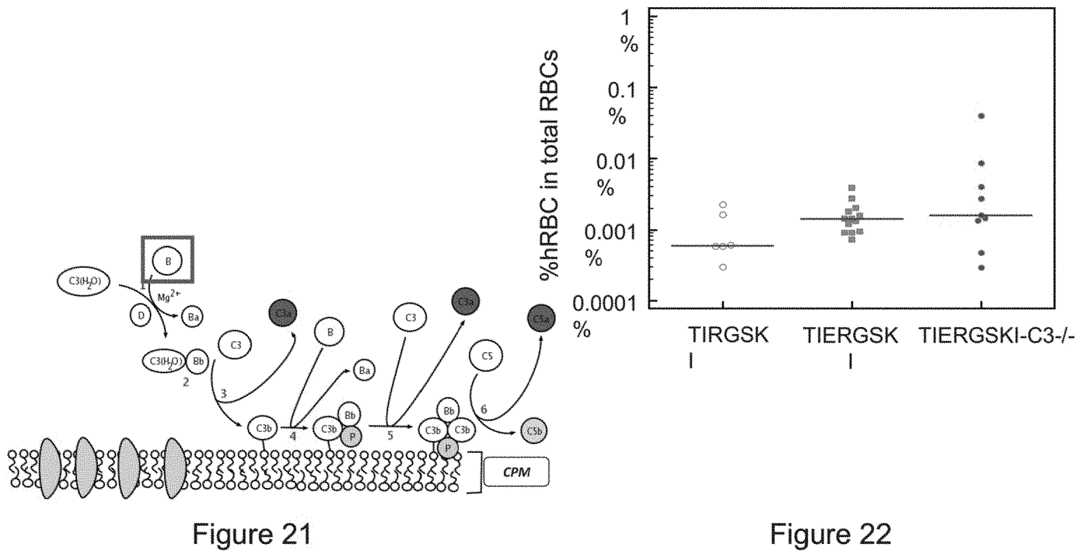

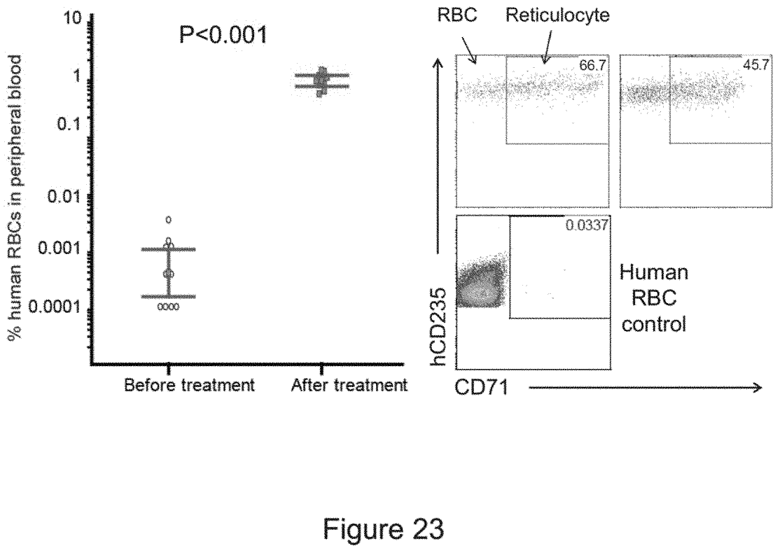

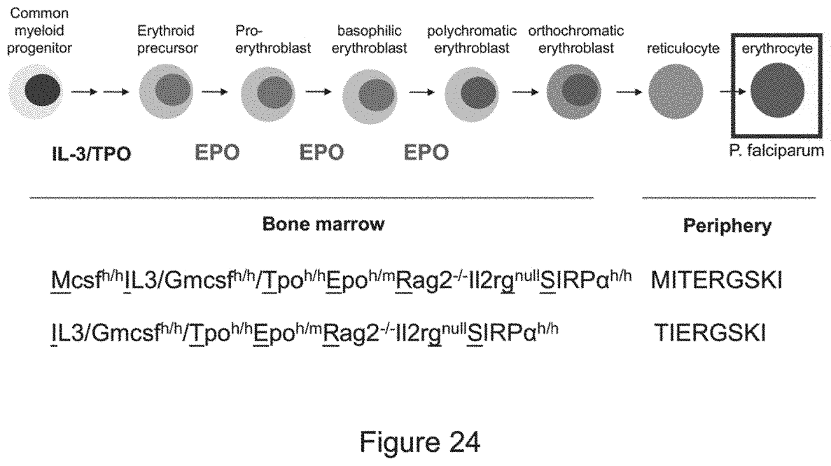

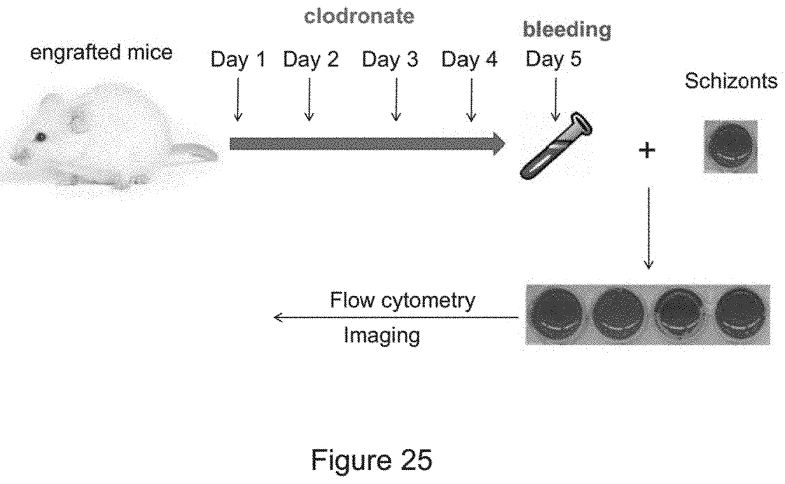

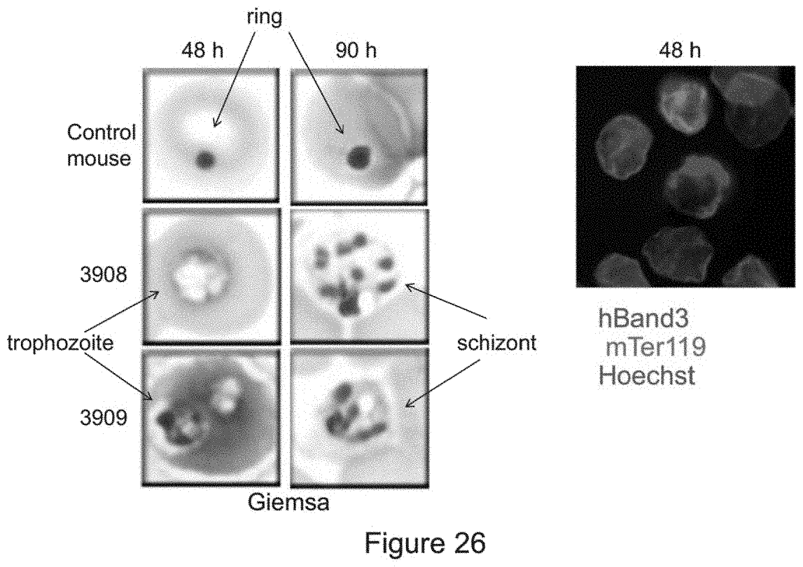

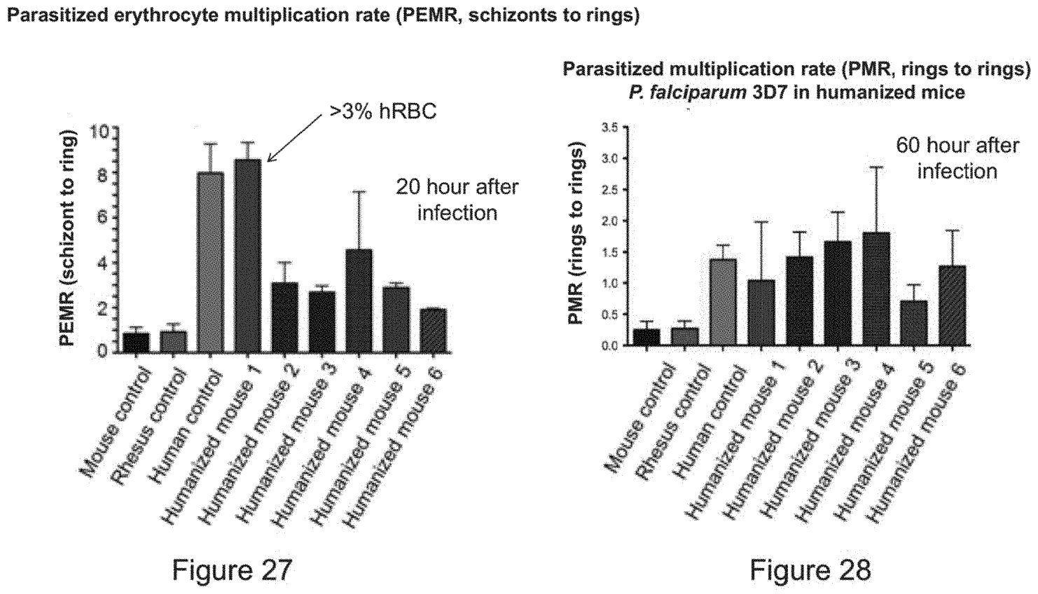

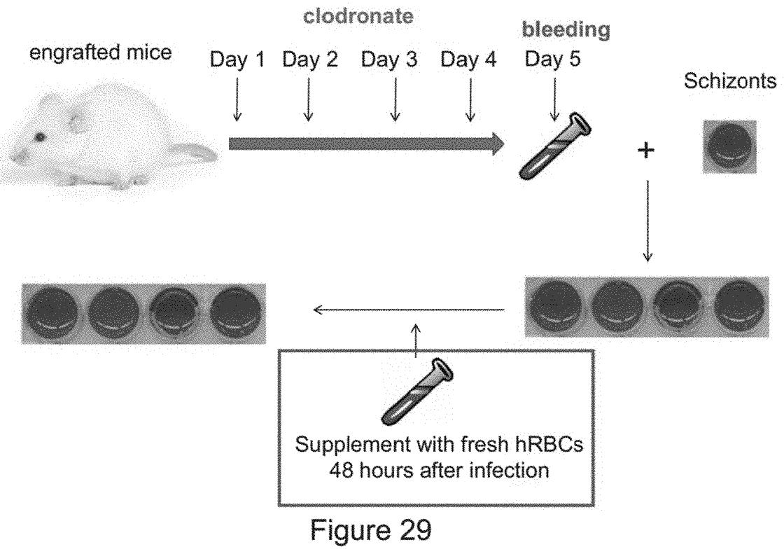

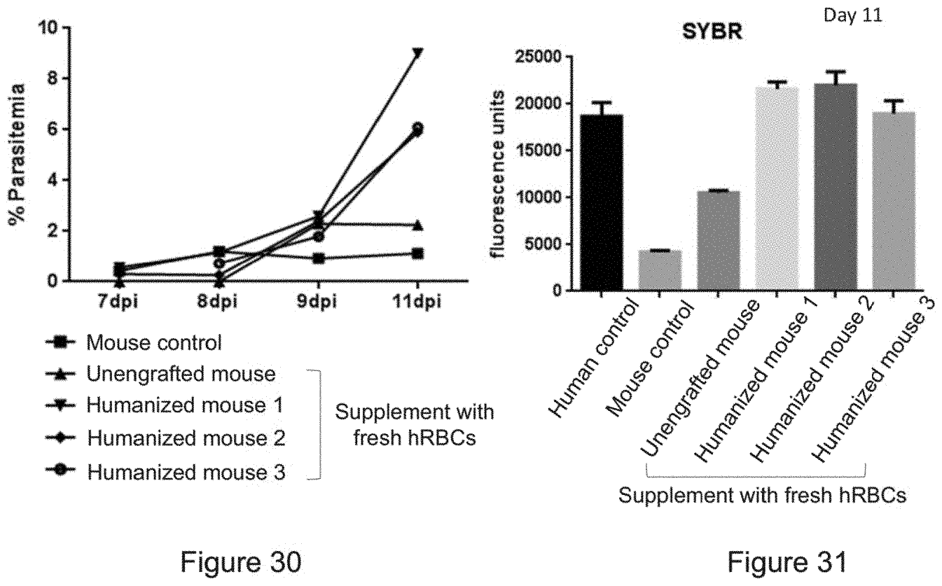

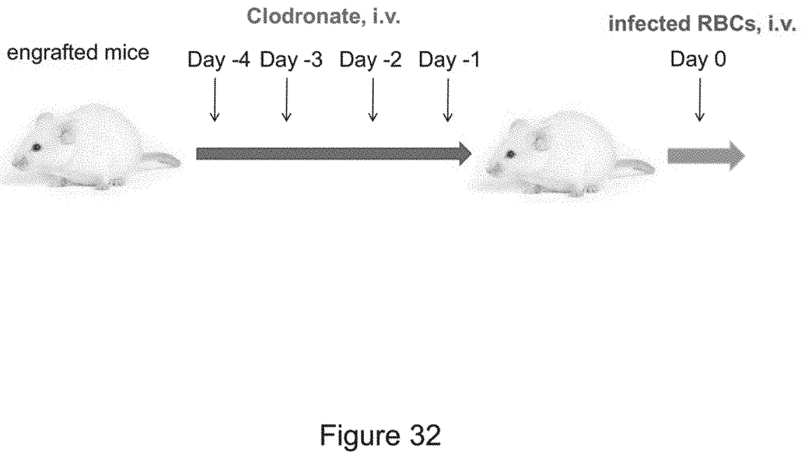

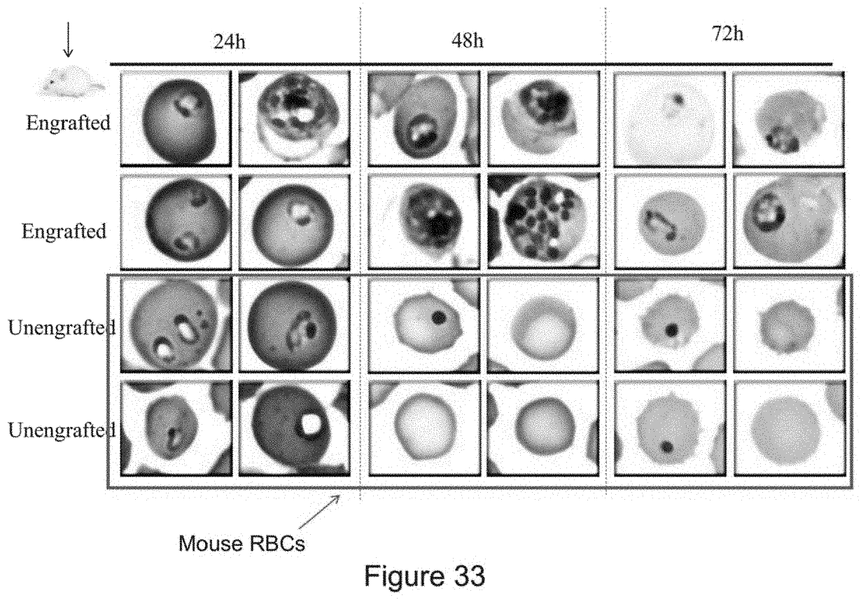

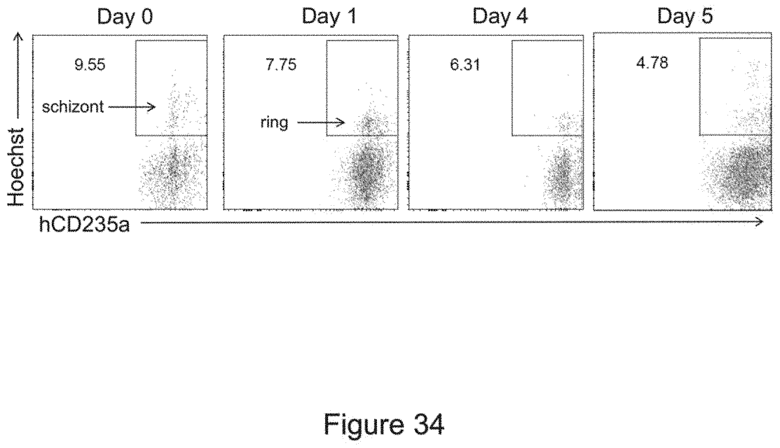

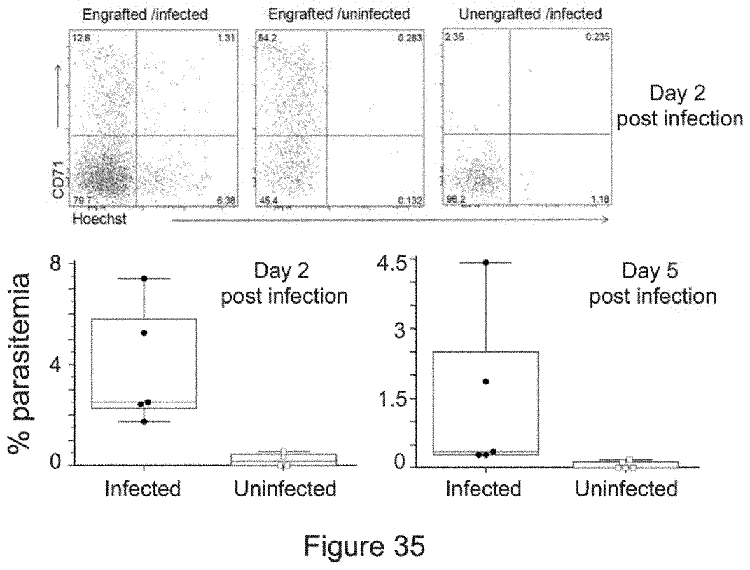

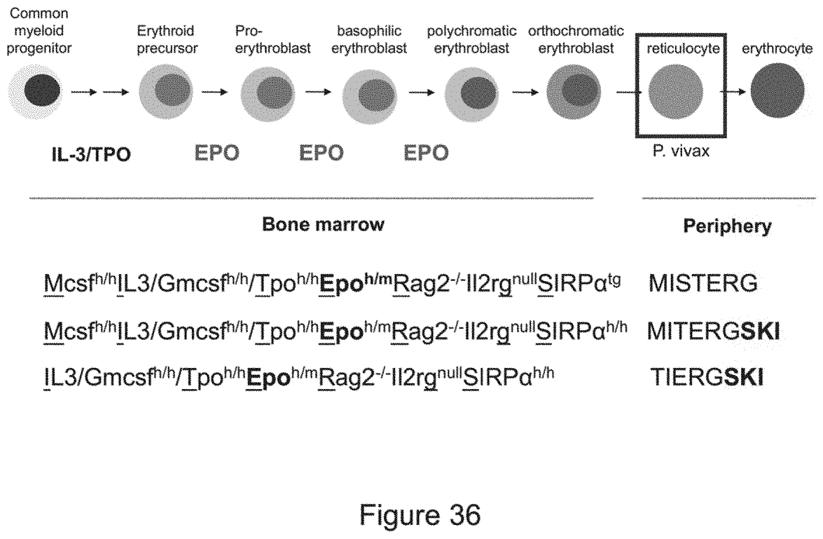

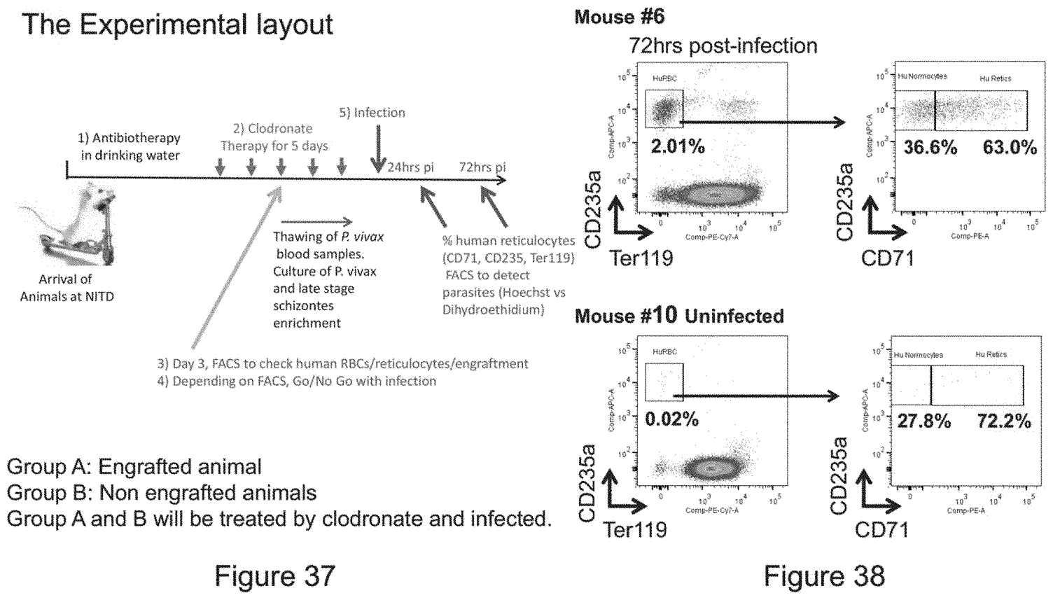

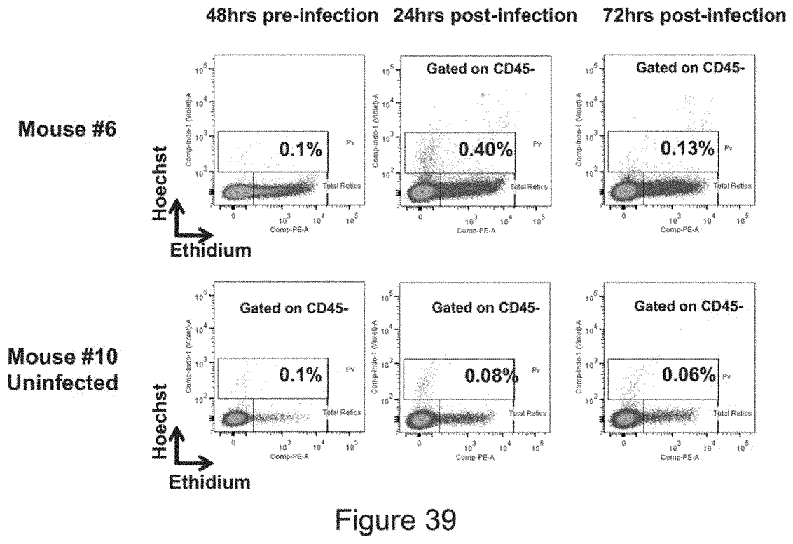

The data presented herein demonstrate that clodronate worked efficiently (IV); up to 2% human red blood cells with a majority of reticulocytes (FIG. 13 through FIG. 39). As shown in FIG. 16, increased frequency of human erythroid cells in the bone marrow was observed but not peripheral blood of hEPO KI mice. It was also observed that complement knockout was not sufficient for the reconstitution of human erythrocytes in the peripheral blood (FIG. 22) and clodronate treatment increased circulating human erythroid cells/reticulocytes in HSC-engrafted TIERGSKI mice (FIG. 23). 7 weeks after HSC-engraftment, TIERGSKI mice were treated with daily retro-orbital injection of 50 ul clodronate liposome for five consecutive days. Frequency of human CD235+ or CD235+/CD71+ cells (human reticulocytes) in the peripheral blood before or after treatment was measure by FACS.

Not much toxicity was observed, though it was not ideal in the eyes. Further, the present results demonstrate that infection (5/10) with P. vivax can generate invasion of circulating human reticulocytes. Rings were detectable by FACS at 24hrs (2/5), inoculum was on the lower side, and the level of invasion was also low. Moreover, almost no parasites were detected by FACS at 72 hrs. Spleen was observed to be dark in some infected animals (perhaps due to parasite clearance). Inoculum was again on the lower side, level of invasion was low as well, and re-invasion challenging was observed.

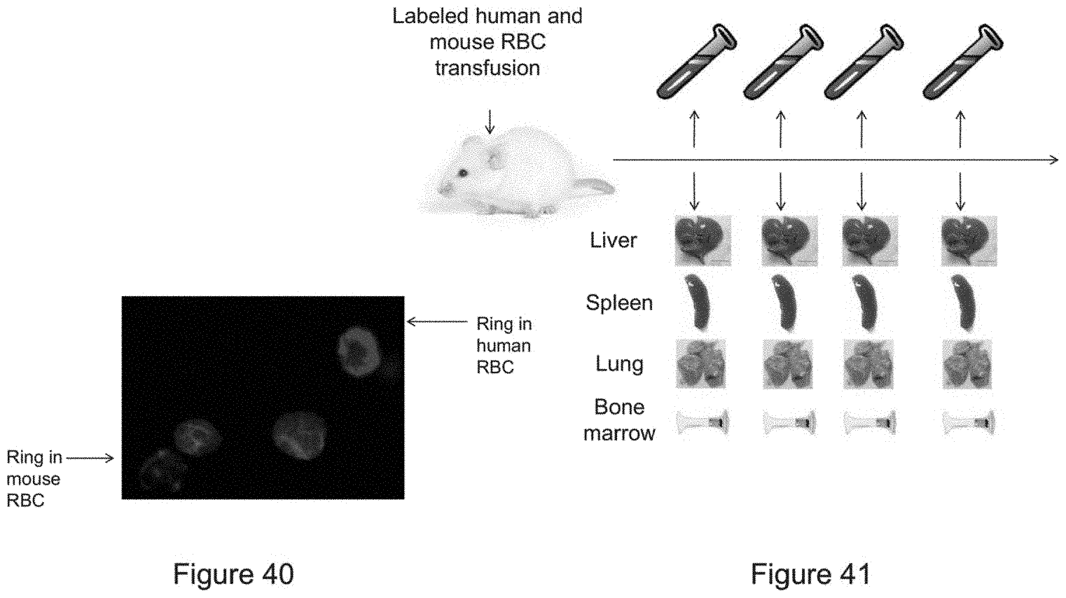

The present data also demonstrated that clodronate treatment resulted in up to 3% human erythrocytes in peripheral blood. Infection could be detected within the first 5 days (P. faciparum) or 24 hours (P. vivax), but the percent parasitemia did not increase (FIG. 40). Although not bound by any particular theory, these results could have been caused by in vivo immune clearance in spleen, or by NK cells (high engraftment level of human CD45+ cells) or by a low frequency of human erythrocytes in periphery. Current mouse model with improving erythropoiesis demonstrated ˜30% human erythroid cells in the bone marrow, ˜1% human RBC/reticulocytes in the peripheral blood, P. falciparum infection, as well as P. vivax infection.

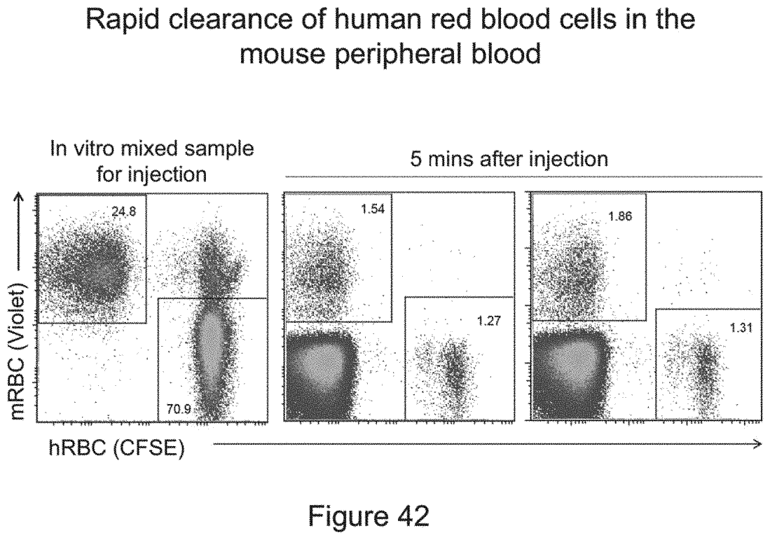

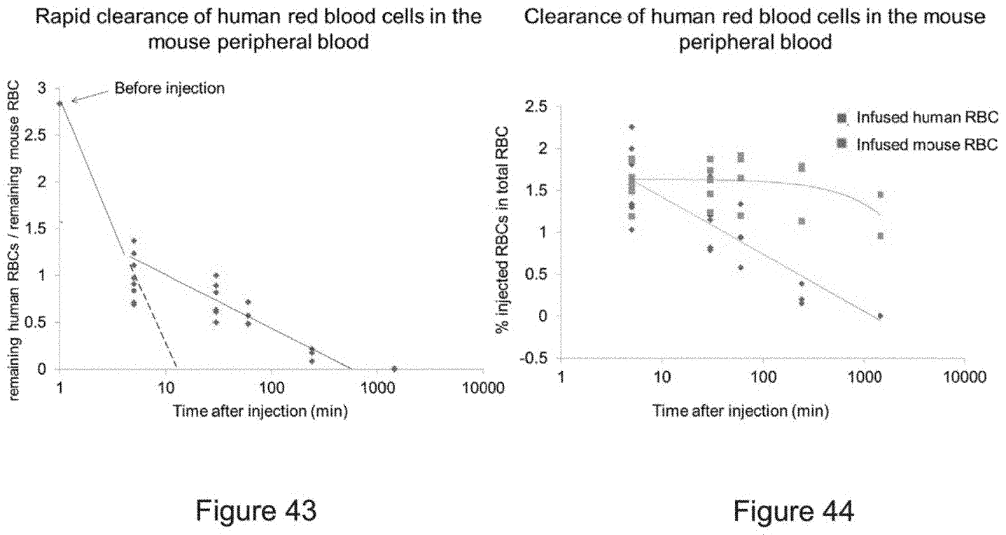

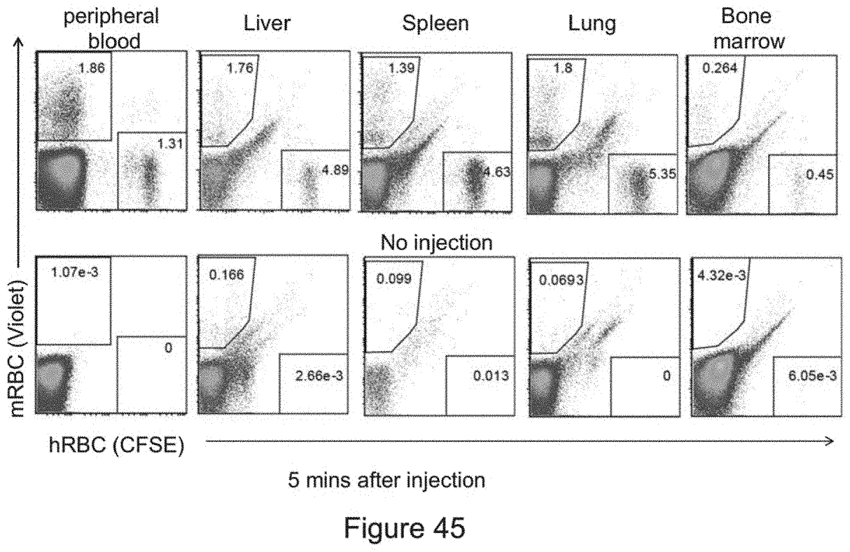

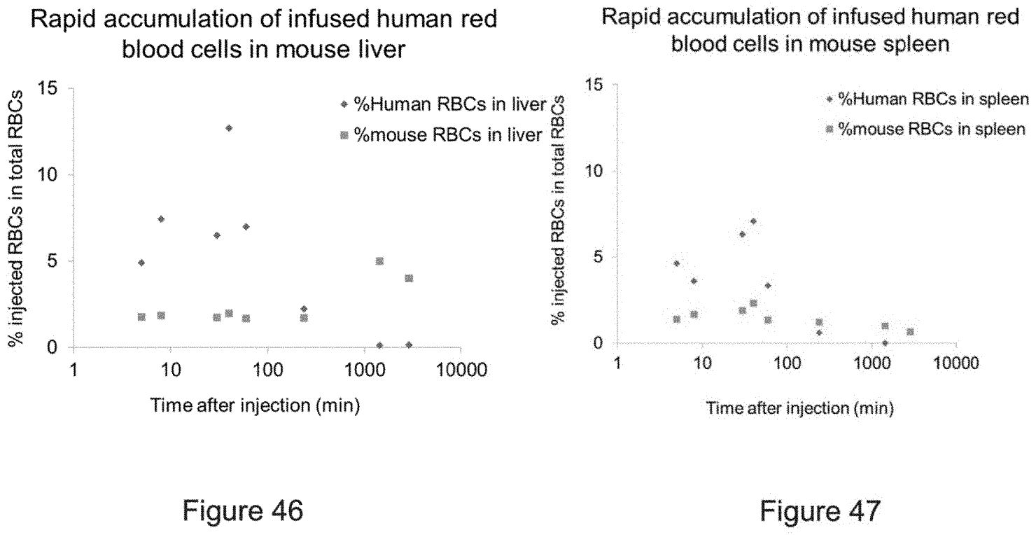

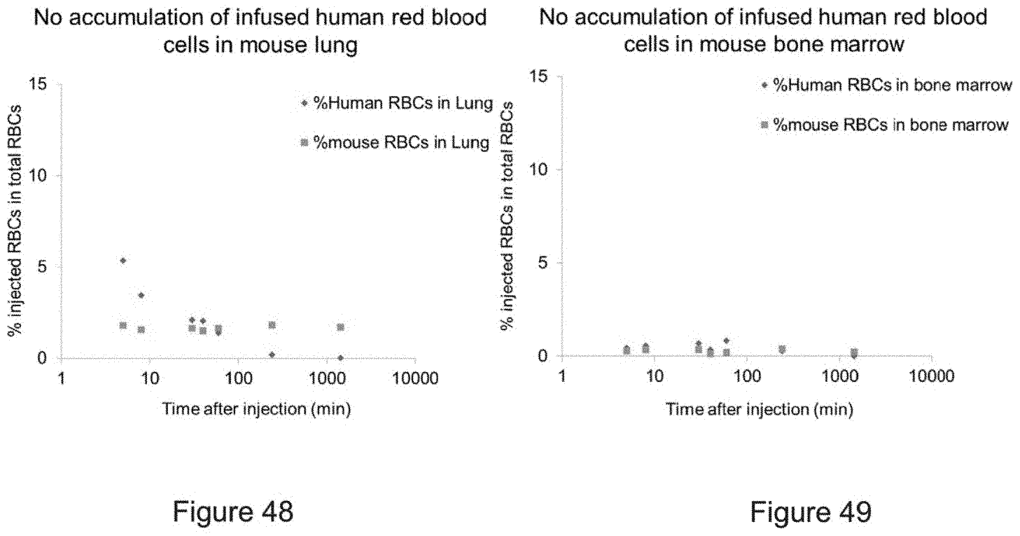

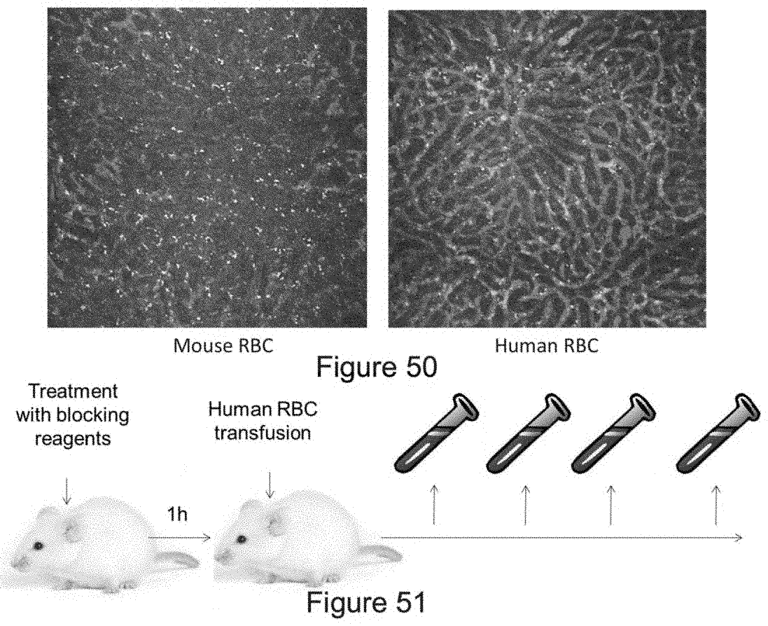

Rapid clearance of human red blood cells in the mouse peripheral blood (FIG. 42 through FIG. 44) and accumulations of infused human red blood cells in mouse tissues, such as liver and spleen (FIG. 45 through FIG. 47 and FIG. 50) were also observed. Moreover, no accumulation of infused human red blood cells was observed in mouse lung and mouse bone marrow (FIG. 48 and FIG. 49). Thus, druglantibody screening was performed to identify mouse liver receptor(s) that mediates human RBC sequestration (FIG. 51).

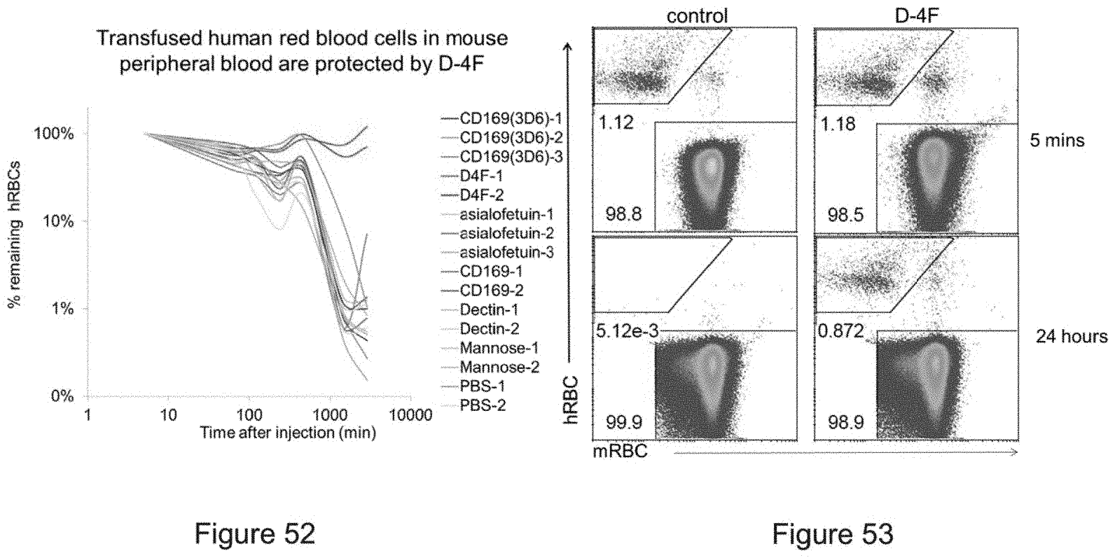

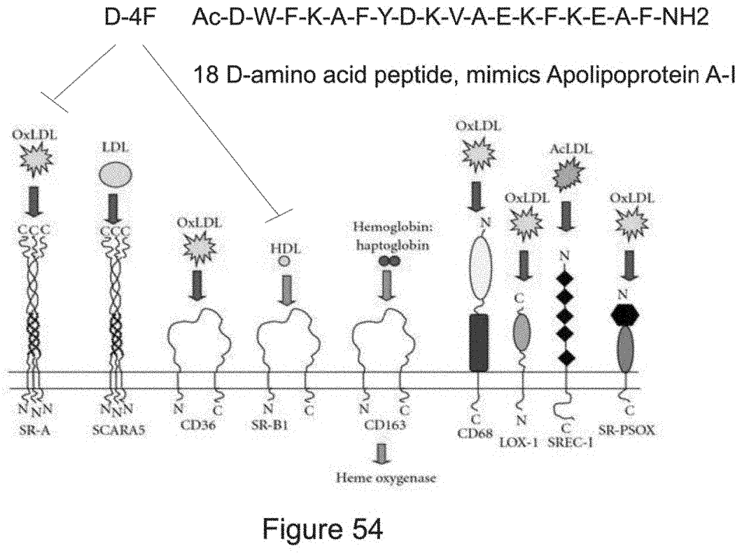

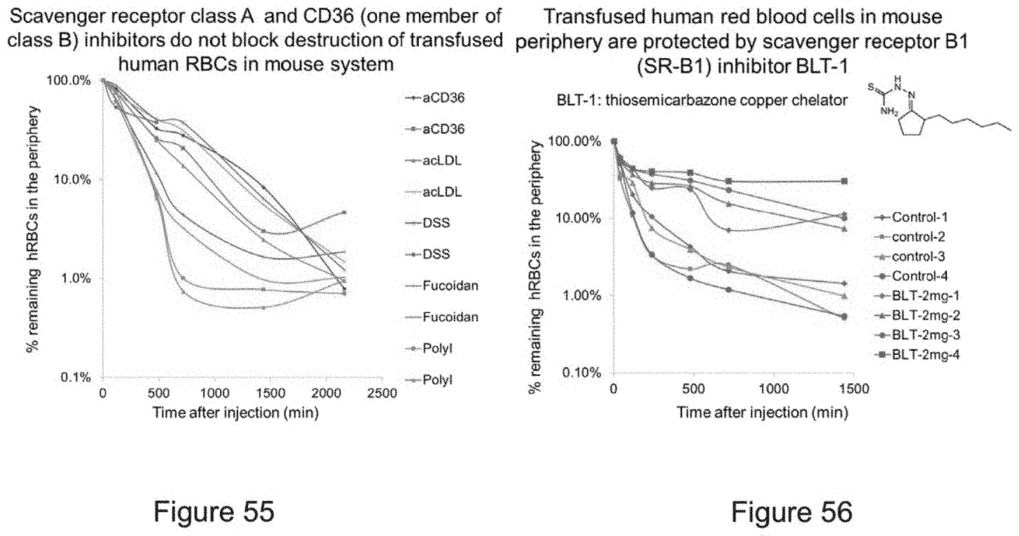

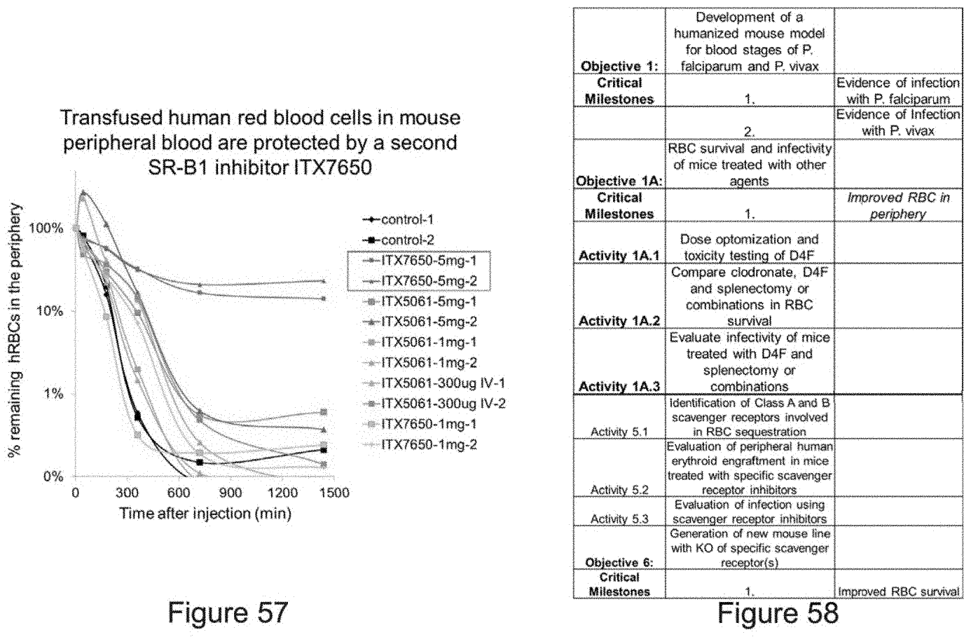

As shown in FIG. 52 and FIG. 53, transfused human red blood cells in mouse peripheral blood are protected by D-4F. Scavenger receptor class A and CD36 (one member of class B) inhibitors do not block destruction of transfused human RBCs in mouse system (FIG. 55) and transfused human red blood cells in mouse periphery are protected by scavenger receptor B1 (SR-B1) inhibitor BLT-1 (FIG. 56) and a second SR-B1 inhibitor ITX7650 (FIG. 57).

In summary, the current mouse model showed improving erythropoiesis, i.e., ˜30% human erythroid cells in the bone marrow and ˜1% human RBC/reticulocytes in the peripheral blood as well as an evidence of Ex vivo and in vivo P. falciparum and P. vivax infection. However, destruction of human erythrocytes in liver, low frequency of human erythrocytes in peripheral blood, single or limited rounds of infection, and the amount of parasitemia during infection could be possible limitation of this model, As such, efforts are directed toward the improvement of the current model to increase human RBCs peripheral blood via combination treatment with clodronate and SR-B1 inhibitor and/or generation of SR-B1 knockout mouse on MITERGSK1 background. Continued animal infection is also studied using P. falciparum (test mouse adapted strain (GSK) at Yale and GSK) and P. vivax (ship mice to NITD) as engraftment improves repeat infection with both pathogens.

### Example 4

### A Blood Cell Matrix for P. vivax in vivo: The “MITERGSK1” Mouse

The pilot study utilized humanized mouse model infect with P. vivax and insured “survival” of circulating human red blood cells with clodronate therapy. The presence of human reticulocytes before thawing P. vivax blood clinical isolates was also determined. If reticulocytes were present, infection was administered with highly concentrated infected RBCs on day 5 (i.e.. Inoculum 2-5 ul of 95% parasitemia in 100 ul sterile PBS) and reticulocytes/infection/parasitemia were then monitored by FACS.

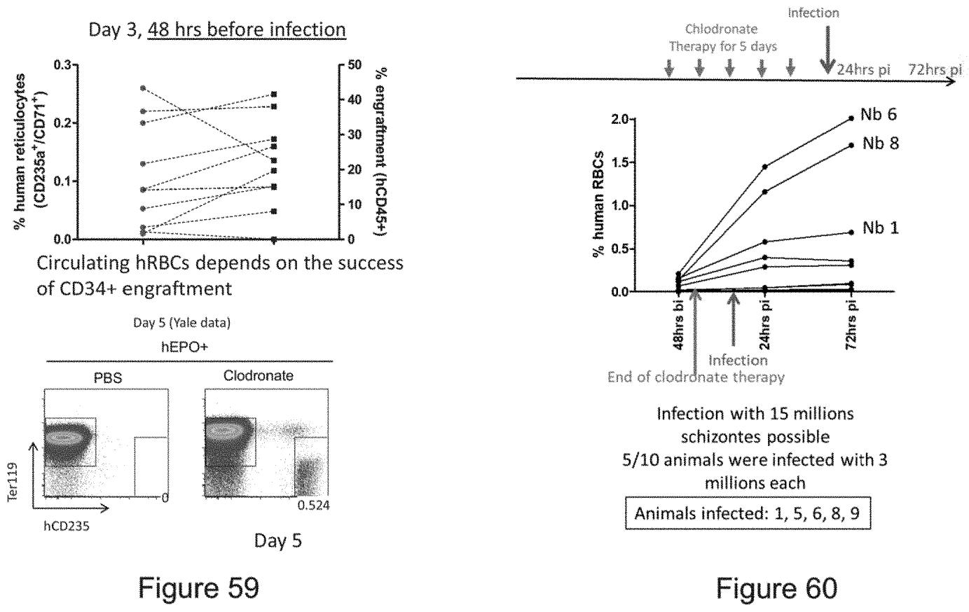

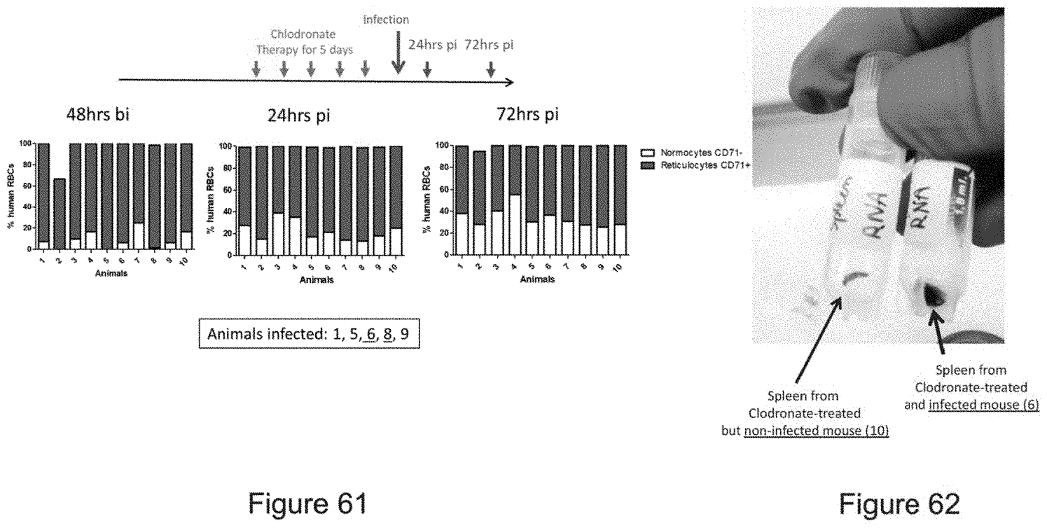

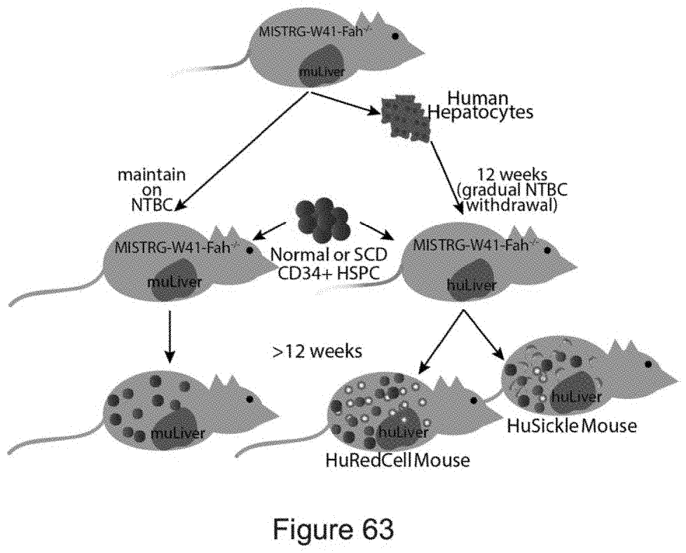

As shown in FIG. 59, circulating hRBCs was shown to depend on the success of CD34+ engraftment. Clodronate progressively limited phagocytosis and most hRBCs in circulation were reticulocytes (FIG. 6G and FIG. 61). Clodronate and infection also impacted the spleen: spleen size reduced in clodronate-treated animals (when not infected; FIG. 62, left) and became darkeri'bigger in infected animals (animal 6, 8) where parasitemia was detected (FIG. 62, right).

As stated in Example 3, clodronate was demonstrated to work efficiently (IV): up to 2% human red blood cells with a majority of reticulocytes and not much toxicity was observed, though it was not ideal in the eyes. Thus, the presence of parasites in all infected animals at 72 hrs post infection is being addressed via (RT)-PCR blood(spleen) (Pvs25/RNA18s). Parallel experiments to monitor clodronate versus antibodies are also performed via anti-CSF1R Abs (phagocytes)+anti-CD56 Abs (target NK), (neutrophils), and IV for clodronate. Animals with highest percent of human reticulocytes are selected and hRBCs is checked only at the end of clodronate therapy (no need anymore for thawing parasites). Modify kinetic of the experiments are also performed via FACS (and smear/thick blood film) at 24, 48 and later time points (up to a week post infection). Splenectomized animals are also examined. Further, extend parasite growth is determined by taking out blood for culture of human reticulocytes in vitro using stand-by clodronate/antibody-treated animals for supply of human reticulocytes.

### Example 5

### MDS Anemia in a Human RBC Competent Humanized Mouse Model

The murine host has remained a readily available and ethically acceptable model for the study of human diseases and therapeutic testing. Immunodeficient mouse models support engraftment of human h.ematopoietic stem cells (HSC) but with limitation in efficiency and mature lineage representation. Herein combined knockin of several non-crossreactive human cytokines (M-CSF, IL3/GM-CSF and Thrombopoietin) into the corresponding murine loci in the SRG strain—in short termed “MISTITRG”—has enhanced engraftment and maintenance of human HSCs with multi-lineage differentiation. The lack of the corresponding murine cytokines in MISTRG mice compromises murine stem cell retention in the niche, which allows human HSC to engraft without irradiation and its accompanying morbidities. This is even more pronounced in mice that carry mutations of the murine stem cell factor receptor c-kit. Interestingly, c-kit mutant immunodeficient mice also show robust human umbilical cord blood-derived erythropoiesis (huEP). Although not bound by any particular theory, it is hypothesized that introduction of the c-kit mutation would further enhance huEP in MISTRG mice and obviate the need for irradiation and its associated bone marrow microenvironment (BME) toxicity and inflammatory response.

Despite robust HSC engraftment and myelo- and erythropoiesis in BM, all humanized immunodeficient mouse models lack mature human RBC, platelets, and other cells in peripheral blood. With universal absence of a murine adaptive immune system, the culprit is likely the innate immune system. Phagocytic tolerance is achieved in part by engagement of the SIRPα receptor on macrophages (MΦs) by ubiquitously expressed CD47, leading to the delivery of a “don't eat-me” signal. While humanization of the SIRPA gene has led to enhanced overall engraftment, it appears to be insufficient to protect cells in circulation. Additional phagocytic tolerance can be achieved by eliminating recipient phagocytic cells altogether by application of clodronate-containing liposomes. Yet liposomal clodronate treatment is toxic with significant mortality and abrogates murine and human phagocytes. Macrophages play a central role in erythropoiesis. They provide proliferative signals to the earliest erythroid progenitors and drive terminal differentiation via direct cues. In addition to their pro-erythroid role in the bone marrow, MΦ in the spleen and liver repair damaged RBC and eventually take senescent RBCs out of circulation. A solution is needed that modulates specifically murine phagocyte-huRBC interactions, that does not cause undue toxicity, and that is long-lived.

In recent years humanization of other organs has served to study human physiology and disease. Deletion of fumarylacetoacetate hydrolase in the Rag−/−I12rγ−/− background has allowed humanization of the liver and served to study diseases, such as malaria. The liver is the site of synthesis of numerous proteins, some of which directly impact hematopoiesis and blood cells, such as complement. It was determined that the mouse liver is the major site of huRBC destruction and that murine complement may contribute to huRBC destruction and, without bounding to any particular theory, it was hypothesized that replacement of the liver with human hepatocytes may ameliorate erythropoiesis and PB RBC persistence.

To date no cure exists for myelodysplastic syndrome (MDS) other than allogeneic stem cell transplantation, a modality not available to the majority of patients due to their age or comorbidities. Novel therapeutics are direly needed in a disease with continued poor outcomes. Studies in primary MDS in vitro are limited due to the inability to grow MDS cells in the culture dish. The heterogeneity of the disease demands trials with large patient numbers to advance therapeutics. A pre-clinical model could greatly expedite translation into the clinic and aid in biomarker development to target the appropriate patient population that could best benefit from a specific treatment,

A highly efficient and improved MDS humanized mouse model that allows study of patient derived MDS bone marrow stem cells in the live mouse context was recently reported. MDS is a complex disease of the blood stem cell that is highly variable between patients and difficult to study as blood stem cells cannot be grown in the culture dish. Thus, mouse growth factors were replaced with human growth factors in immunodeficient mice, which greatly increased the ability of MDS stem cells to grow and give rise to the different blood cells. However, these blood cells did not survive or circulated in the mouse blood, most likely due to remaining mouse immune cells that can eat human blood cells.

For this reason, studies were directed to overcome this shortcoming by also replacing almost all of the mouse liver cells with human liver cells. The present data confirm that human liver cells fully support the mouse's life but (unlike mouse liver cells) do not make certain proteins that recognize human red blood cells as foreign that would lead to their destruction. This model is then optimized and studied for the interplay between human red cell precursors and their support cells in the bone marrow in healthy and MDS bone marrow stem cell engrafted mice. Lastly, tests are conducted to assess novel treatments and combination regimens in MDS blood formation.

Cytokine humanization in immunodeficient mouse models has represented a major advance for the enrollment and lineage representation of MDS primary cells in xenotransplantation studies. It was previously shown that all subtypes of MDS efficiently engraft and give rise to clonal hematopoiesis with faithful representation of dysplasia, clonal representation and evolution, and serial MDS stem cell transplantation (Song et al., 2019, at. Commun., 10:366). However, all models to date fail to sustain mature myeloid cells, and in particular human red cells (RBC) in peripheral blood (PB). Human RBC are rapidly removed from circulation most likely due to the remnant murine innate immune system. Abrogation of murine phagocytes via injection of liposomal clodronate spares human RBC in circulation, but only to a small percentage and only transiently. The precise mechanism of human red cell destruction remains unknown, and to date no viable solution with persistence of human PB RBC has been identified.

MDS anemia is variable and its origin complex. Cell intrinsic factors, such as recurrent chromosomal aberrations and mutations, and cell extrinsic factors, such as cytokines and signaling molecules emanating from the bone marrow microenvironment, have been identified. Several therapeutics, such as the IMIDs ire 5q- syndrome and Tilifb inhibitors, are currently in the clinic. Pre-clinical in vivo studies of human MDS with efficient modeling of erythropoiesis and peripheral blood 1?,.13C as readout could significantly expand the therapeutic armamentarium and ideally lead to expedited. translation of combination therapies into the clinic.

The present data demonstrate that the mouse liver is the major site of human RBC destruction. Replacement of the mouse liver, via deletion of the fumarylacetoacetate hydrolase (Fah) gene and staged regeneration of the damaged murine hepatocytes with transplanted human hepatocytes (HuHep), significantly increases human RBC in circulation for the duration of the mouse's lifespan. :Introduction of the c-kit “W41” (V831M) mutation that compromises murine HSPCs niche retention into MISTRG mice eliminates the need for irradiation, and improves human EISC engraftment and erythropoiesis.

To develop a xenograft host fully competent to model human MDS heniatopoiesis and erythropoiesis, from the HSC to mature RBC in circulation, the following approach is followed.

Section 1: Humanization of the Host's Liver in MISTRGW41Fah Mice and Determination of the Mechanism by which Human RBC and Platelets are Spared in Circulation

Human hepatocytes are engrafted into MISTRGW41Fah livers in order to confirm humanization of the host's liver via serial measurements of human albumin. When >80% liver humanization is achieved, primary adult CD34+ HSPC is engrafted and the engraftment is monitored. Erythropoiesis, the erythropoietic niche, RBC maturation and survival in both liver-humanized and control MISTRGW41Fah mice are assessed.

### Section 2: Model MDS in HuHepMISTRGW41Fah Mice to Determine Patient-Mouse Correlation and Test MDS Treatments Targeted at Overcoming MDS Anemia

MDS CD34+ of distinct subtypes are engrafted with particular attention to 5q- and SF3B1 mutation, and age matched control CD34+are also engrafted into liver-humanized HuHepMISTRGW41Fah mice and correlated with the patient's phenotype and genotype. Response of human PB RBC to TGF β inhibition (TGF-β superfamily ligand traps) is assessed and MDS erythropoiesis, dysplasia, and mutational profiles are characterized.

Previously reported data and above described results have shown that combined knockin of several non-crossreactive human cytokines (M-CSF, IL3/CiM-CSF, and Thrombopoietin) into the corresponding murine loci in the SRG strain, in short termed “MISTRG”, has enhanced engraftment and maintenance of human h.ematopoietic stem cells (HSCs), and in particular of MDS HSCs, with multi-lineage differentiation and. faithful replication of the mutational landscape and dysplastic changes (FIG. 6E through FIG. 6I and FIG. 8A through FIG. 8C) (Song et at, 2019, Nat. Commun., 10:366; Rongvaux et al., 2014, Nat. Biotechnol., 32:364-372). MISTRG mice are the only mouse model that expresses human M-CSF, resulting in full maturation of human monocytes/macrophages. As a result, MISTRG, but not NSG, mice formed erythroblastic islands (EBI) with central human CD169+ macrophages (FIG. 8A through FIG. 8C). Engraftment in MISTRG and other mouse strains require irradiation for efficient engraftment, which damages the BME and induces inflammation, Mutation of the murine c-kit receptor rendered endogenous murine HSPC partially resistant to muScf and provided human HSPC a competitive advantage, allowing engraftment without irradiation (Waskow et al., 2009, Nature Methods 6:267-269; Cosgun et al., 2014, Cell Stem Cell 15:227-238). Similarly, the “erythropoietic niche” became receptive to human erythroid progenitors with robust establishment of UCB-derived (Yurino et al., 2016, Stem Cell Reports 7:425-438; Rahmig et al,, 2016. Stem Cell Reports 7:591-601) and adult erythropoiesis (FIG. 7A and FIG. 7B). However, despite significant BM erythropoiesis, human RBC are completely absent in the murine host. When fluorescently labeled human and murine RBC were injected into MISTRG mice, huRBC were rapidly cleared from circulation (FIG. 10A). Intravital imaging on liver (FIG. 10B) and other organs revealed significant sequestration of CFSE labeled human (FIG. 10B), but not murine (FIG. 10C) RBC in the host's liver vasculature,

Deletion of the Fah gene in MISTRG mice allows humanization of the liver via gradual NTBC water withdrawal after engraftment of human hepatocytes via splenic vein injection. Human albumin levels steadily rose (FIG. 11A) with achievement of levels >2 mg/mL at 10 weeks, that were reflective of 80% liver humanization. “Liver-humanized” (HuHep) MISTRGFah. mice efficiently engrafted with CD34 cells and were fully viable >16 weeks post transplant (FIG. 11B). Of note, HuHepMISTRGFah and MISTRGW41 showed improved BM erythropoiesis compared to MISTRG mice (FIG. 10A). However, only engrafted HuHepMISTRGFah mice had significant, up to 10%, human RBC in circulation (FIG. 11F and FIG. 11G red circles). While MISTRG and MISTRGW41 resulted in coating of all BM erythropoietic cells with murine complement C3, this was absent in HuHepMISTRGFah mice (FIG. 11F pink circle). Since the liver is the predominant site of human RBC sequestration, the liver was analyzed tier presence of murine vs human phagocytes. In HuHepMISTRGFah mice huCD68+ human macrophages fully replaced murine F4/80+ macrophages (FIG. 11H). Of note, peripheral blood huRBC in HuHepMISTRGFah mice were fully enucleated (assessed via Hoechst staining) and consisted of a mixture of mature RBC and reticulocytes (assessed via thiazole orange staining of RNA).

Together these data provide strong evidence that, via mutation of the c-kit receptor and humanization of the mouse liver, one can achieve significantly improved overall erythropoiesis and full maturation of human RBC.

These data also demonstrate that 1) the c-kit mutation allows engraftment without irradiation and opens the erythroid niche to human erythropoiesis, that 2) MISTRG mouse BM contains EBIs composed of central huMΦs and human EP (as well as huMΦ and murine EPs and mixed (hu, mu) EBIs), and that 3) humanization of the murine host's liver (>80% human hepatocyte chimerism and >90% survival) allows full erythroid maturation with enucleation and persistence of mature human RBC in circulation.

Several limitations to hematopoietic xenotransplantation have been overcome over the past years. Identification of the critical nature of the SIRPA-CD47 axis has reduced rejection of human cells. Humanization of cytokines by transgenic overexpression has improved engraftment of certain hematologic malignancies but not hematopoietic stem cells. Humanization of cytokines via knockin technology has improved HSC engraftment and development of the human innate immune system. The herein described studies take these advances a significant step further with the result of a human “RBC competent” PDX model.

However, (MDS) hematopoiesis is still assessed in the xenogeneic setting and immune barriers persist in both directions. In addition, while greatly improved, the host's microenvironment continues to be mostly murine; additional modifications may he necessary in the future. These barriers are identified and resolved to advance the model. Replacement of an entire liver, which is complicated but feasible, is such an advance because innumerable proteins, known and unknown, are humanized at once. This is an extraordinary opportunity to identify additional factors critical for HSC maintenance and lineage differentiation. Liver humanization may not be feasible in other laboratories; this particular model's transferability might be limited to more specialized groups.

MISTRG mice do not express human erythropoietin; human erythropoietin were tested in vivo without benefit. However, their addition is considered if it becomes evident that erythropoietic defects can be secondary to Epo deficiency. Combination of erythropoietin with TGF-β inhibitors is attractive given the distinct mechanisms and sites of action.

It is possible that while healthy CD34+ engrafted mice give rise to mature RBC in circulation, MDS engrafted mice may not. in that case, drug treatments, such as with TGF-β pathway inhibitors, are expected to have a significant effect. It may be, however, that MDS erythropoiesis cannot overcome the creno-barrier despite liver humanization. These studies identify the first modifications to murine macrophages that may further ameliorate innate immune tolerance. Such modifications may be beneficial in addition to liver humanization or in place of liver humanization in conjunction with other genetic alterations.

The immediate goals of the present studies are to take the MDS xenografts model to the next level by overcoming barriers to human RBC (and other mature myeloid cell) maturation and persistence in circulation. This model is used to understand MDS pathology with particular focus on MDS anemia; the interplay between MDS erythropoiesis and the central macrophage is specifically addressed. In addition, the utility of this model to determine effectiveness of therapeutics that specifically target MDS erythropoiesis is assessed.

Ultimately, the present studies provide a route toward further understanding of MDS clonality and its effect on treatment outcomes, understanding of the role of the microenvironment in MDS disease initiation and progression, and development of new treatment approaches and therapeutics addressing defective stem cell maturation as well as clonal evolution and progression to leukemia. This model's utility extends beyond MDS and potentially inform “pre-cursor diseases”, such as clonal hematopoiesis, and results in preventative measures.

Humanized immunodeficient mice, named MISTRG, that support far superior engraftment of MDS stem cells, clonal representation, and representation of dysplasia were generated. The herein described xenotransplantation model carries great potential to advance such research and in particular pre-clinical therapeutic studies. The present data provide an evidence of feasibility of this approach and have assembled a highly complementary team of scientists to advance MDS research. The herein described model is highly innovative and opens up innumerable avenues of study. The development of the model and in vivo study of patient derived MDS in immunodeficient mice require expertise in several areas, such as in i) work with immunodeficient mice and dedicated animal care staff and veterinary care, in ii) hematopoiesis and MDS specifically, iii) expertise in inflammatory signaling, and iv) sophisticated molecular biology techniques.

The materials and methods employed in these experiments are now described.

### Materials and Methods of Example 5

Section 1: i) Kinetics of RBC survival in HuHepMISTRGW41Fah mice compared to non-human liver mice are determined by injecting CFSE-labeled human and violet-labeled murine RBC and measuring their persistence in circulation (FIG. 10A). ii) The intra-vital imaging of the liver of injected mice is repeated to determine huRBC vs muRBC fate in the humanized vs murine liver (FIG. 10B and FIG. 10C).

As shown in FIG. 11A through 11H, HuHepMISTRGFah mice were successfully engrafted with healthy human CD34+ cells. These data suggested that human EP and RBC in bone marrow and blood of engrafted buHepMISTRGFah mice lack muC3 coating and RBC fully mature and egress to the iii) Human and murine complement is measured via Elisa and muC3 ins huC3 coating of hu vs muRBC compared to control non-human liver mice and iv) kinetics of survival of endogenously produced human and marine RBC are determined.

The herein described data also show the ⁻formation of fully human as well as mixed EBIs in CD34+ engrafted HuHepMISTRGFah mice. Flow-sorting (slow-flow, 100 um nozzle) of EBI is established (FIG. 8A through FIG. 8C) from both primary human BM samples and engrafted mice BM for v) single cell cytokine chip and vi) scRNAseq analysis,

Liver humanization is expected to improve overall human erythropoietic progenitor engraftment, full huRBC maturation, huRBC persistence in circulation; single cell analysis of huEBI is also expected to shed light on huMΦ-EP interactions supportive of (huMΦ-huEP) and potentially detrimental to (huMΦ-muEP mixed EBI) erythropoiesis. Studies in Section 1 enable optimization of this complex model.

Section 2: Analysis is focused on low-grade MDS from patients with/without anemia, with/without 5 q and SF3B1 mutations; CD34+ cells from age-matched healthy donors serves as controls.

In engrafted HuHepMISTRGW41Fah mice i) full characterization of MDS compared to healthy hematopoicsis and specifically erythropoiesis is performed as in Song et al. and in Section 1. ii) BM cytokine expression is also determined. iii) The presence and composition of MDS vs healthy EBI is assessed via flow imaging using the Amnis ImageStream imaging flow cytometer. iv) EBI is sorted, and cytokine secretion and RNA expression is assessed as in Section 1 comparing MDS EBI to healthy EBI.

As proof of principle for the validity of therapeutic testing in HuHepMISTRGW41Fah mice, an access to enasidenib (IDH2mut inhibitor) and TGF-β superfamily ligand traps (luspatercept) for in vivo use is evaluated. v) Effects of IDH inhibition on MDS h.ematopoiesis (as in Song et al) and effects of luspatercept on MDS erythropoiesis in vivo are determined,

All mouse models are available, techniques established, and reagents including numerous primary MDS BM samples at hand. All experimental techniques were optimized, allowing high reproducibility of data and statistical significance.

The herein described studies are highly sophisticated and combine several creative and novel approaches to advance the state of the art MDS preclinical model, and further the current understanding of MDS anemia.

The present data suggest that what appeared to be an unsolvable obstacle was overcome: the immune destruction of mature human RBC in the murine host circulation. Replicability of these findings, such as the particular characteristics of MDS with isolated 5 q and SF3B1 mutations (with ring sideroblasts) compared to others, confirming validity of the model, is expected.

Exploration of the erythroblastic island, both phenotypically as well as functionally (via RNAseq, scRNAseq, and the unique cytokine chip), is expected to shed light on both cell-intrinsic and cellextrinsic mechanisms of MDS anemia. The application of known and brand new clinical therapeutics in this model validates this MDS model for pre-clinical therapeutic studies and allows assessment of combination regimens and high-throughput in vivo assessment of effects of mutational complexity on therapeutic response.

### Example 6

### Establishment of an NAHA). Animal Model in a Humanized Liver

The other great value of this model system, through combining human liver engraftment with human blood cell engraftment is the enablement of the study of human inflammatory diseases in the liver. These diseases are caused by the infiltration of human immune cells into, or the activation of resident human immune cells in the liver. These diseases involve fatty liver disease, non-alcoholic and alcoholic steatohepatitis. These diseases progress to liver cirrhosis and liver cancer; again no model is available for this. Another related important need, which the herein described models address, is that they can be infected with hepatitis viruses including HBV and HCV. There are no other known effective models where this can be done.

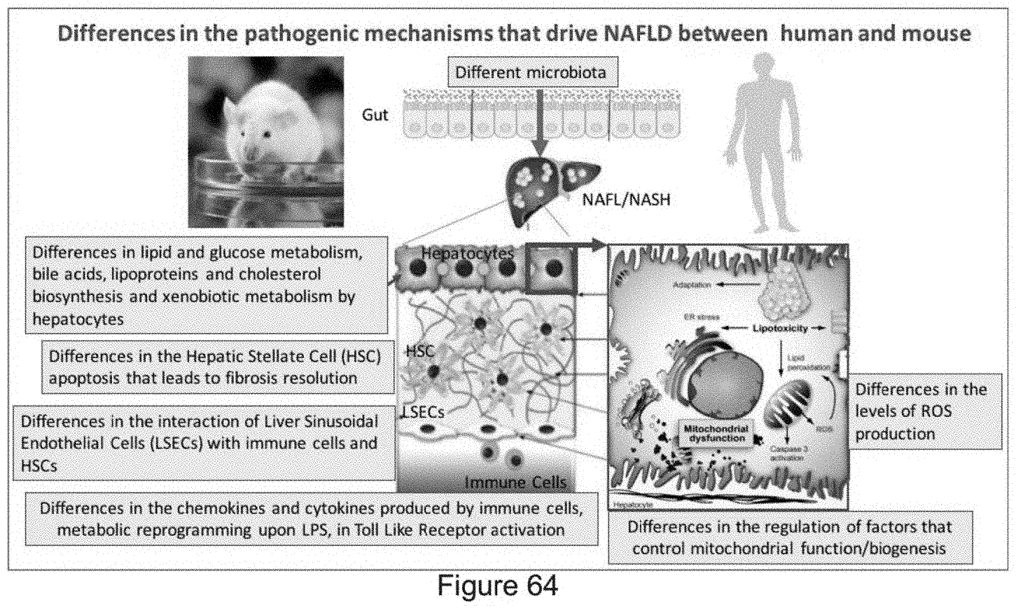

Non-Alcoholic Fatty Liver Disease (NAFLD) is rapidly becoming the most prevalent liver disease worldwide affecting up to 20-35% of the general population (Angulo et al., 2002, N Engl J Med., 346:1221-1231; Ascha et al., 2010, Hepatology, 51:1972-1978). A sizable minority ofNAFLD patients develop Non-Alcoholic Steatohepatitis (NASH) which is characterized by inflammatory changes that can lead to progressive liver fibrosis, cirrhosis, and Hepatocellular Carcinoma (HCC) (Ratziu et al., 2010, J Hepatol., 53:372-384). Currently NASH-related HCC is the fastest growing indication for liver transplantation in HCC candidates (Cholankeril et al., 2017, World J Hepatol., 9:533-543). The proportion of NAFLD and of HCC related to NAFLD are anticipated to increase exponentially the next years but at present there is no approved therapy for NAFLD. This progression of fatty liver to NASH and HCC involves complex immune-stromal cell interactions, metabolic alterations and an important role of the gut microbiota (Friedman et al., 2018, Nat Med., 24:908-922), all currently studied in mouse models. However, humans and rodents are significantly different in terms of hepatic glucose/lipid metabolism, immune system function and gut microbiota composition (Ellis et al., 1998, Hepatology, 27:615-620; Ellis et al., 2013, PLoS One, 8:e78550; Chandrasekera et al., 2014, ALTEX, 31:157-176; Vijayan et al., 2019, Redox 22:101147; Zschaler et at., 2014, Crit Rev Immunol., 34:433-454; Kawada et al., 2006, Gut, 55:1073-1074; Rakhshandehroo et al., 2009, PLoS One, 4:e6796; Hui et al., 2018, Hepatology, 68:2182-2196) (FIG. 64). Thus, there is an urgent need to develop novel humanized mouse models in which both immune system and liver stromal cells as well as the gut microbiota are human. Since the progression of NAFID to NASH and HCC is the result of a cross-talk among tnicrobiota, damaged hepatocytes and stromal cells (hepatic stellate cells, endothelial and immune cells) the study of human cells in vitro/ex vivo does not allow the study of the mechanisms that are involved in this cross-talk. A human liver with human hepatocytes, human immune cells, human endothelial cells and human hepatic stellate cells enables the study of human molecular targets in human liver cells within the pathophysiological context of a liver disease in vivo. This animal model is missing and its development is urgently required.

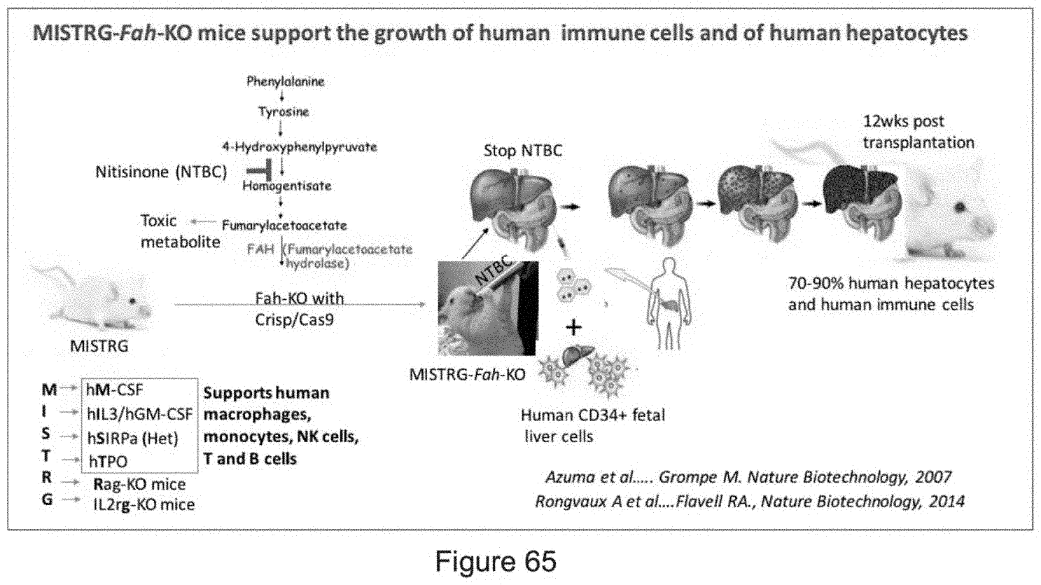

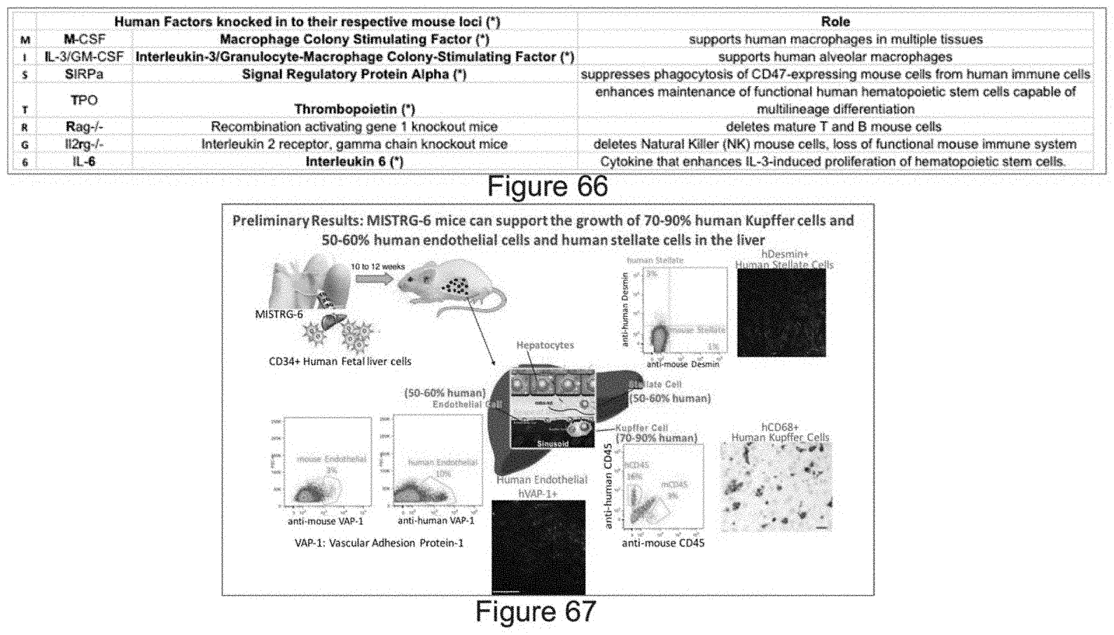

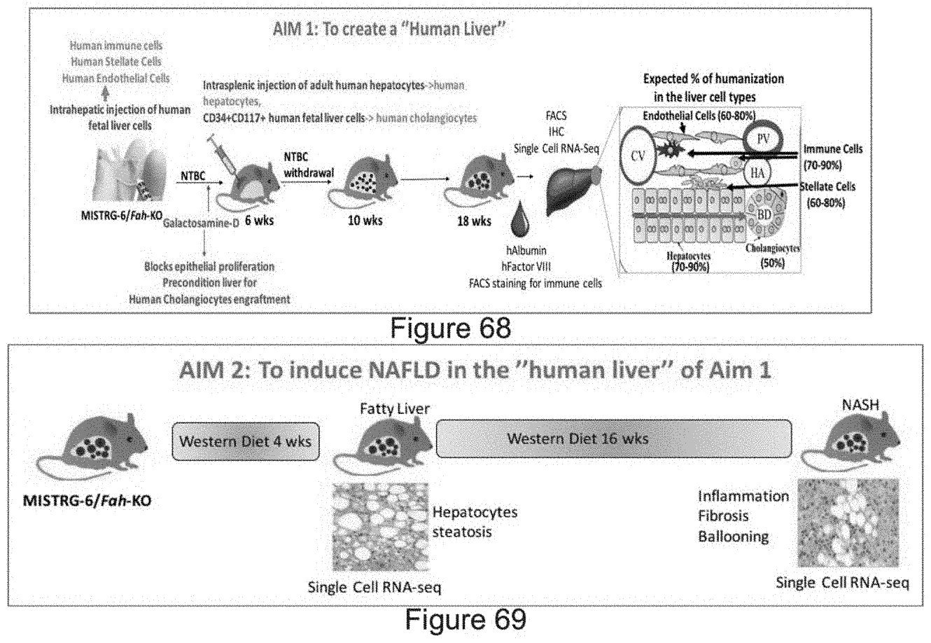

To establish this model, MISTRG/Fah-KO mice were generated (FIG. 65) by integrating two important mouse humanization technologies: 1) MISTRG mice (FIG. 66) (Rongvaux et al., 2014, Nat Biotechnol., 32:364-372) and, 2) Fah-KO mice (Azuma et al., 2007, Nat Biotechnol., 25:903-910). MISTRG mice combine immunodeficiency with humanization of critical factors; MISTING is named for the encoded human proteins that is knock in (FIG. 66). These human factors allowed the robust development of functional human monocytes, macrophages, NK cells, T and B lymphocytes in high percentage (70-90%) after intravenous or intrahepatic transplantation of human heamatopoietic stem cells (CD34+) from human fetal liver. Therefore, this model supported the best human immune system ever achieved (Rongvaux et al., 2014, Nat Biotechnol., 32:364-372). Fah-KO mice were deficient for fumarylacetoacetate hydrolase (Fah) that resulted in the production of the toxic metabotitk. succinylacetone. This metabolite destroyed mouse hepatocytes allowing the repopulation with human liver cells. These mice have been already shown to support the growth of mature and functional human hepatocytes (Azuma et al., 2007, Nat Biotechnol., 25:903-910) and to have a typical human lipoprotein and bile acid profile (Ellis et at, 2013, PLoS One, 8:e78550). MISTRG/Fah-KO mice have been shown to be highly engrafted (up to 90%) with human adult hepatocytes from multiple sources, including liver biopsies as well as with human fetal liver cells that can support the development of a human immune system (FIG. 65). Other studies have shown that human fetal liver cells, support the growth of human cholangiocytes in Galactosamine-D treated mice (Nowak et al., 2005, Gut, 54:972-979) and the growth of human liver endothelial cells (Fomin et al., 2017, Open Biol., 7). However, at present there is no animal model that can support simultaneously human immune cells, human hepatocytes, human liver endothelial cells, human cholangiocytes and human hepatic stellate cells, which is the first section of this study. The second section is to develop NAFLD in the mouse with the human liver.

Section 1: Development of a Mouse with Human Liver That Can Support the Growth of:

Section 1 focuses on the development of a mouse with human liver that can support the growth of: 1) human hepatic stellate cells: the main effector of liver fibrosis in NASH; 2) human liver endothelial cells: an important gate-keeper for immune and hepatic stellate cells activation; 3) human cholangiocytes: the first line of defense of the biliary system against microbial products translocating from the gut to hepatic sinusoids; 4) human hepatocytes and human immune cells: the main effectors in NASH (Rongvaux et al., 2014, Nat Biotechnol., 32:364-372; Grompe et al., 2017, Adv Exp Med Biol., 959:215-230).

In the present studies, MISTRG-6 mice (a modification of MISTRG that express also human IL-6) engrafted with heamatopoietic stem cells (CD34+) from fetal liver intrahepatically was, surprisingly, shown to support the growth of some sub-populations of human liver endothelial cells and of human liver stellate cells. As such, the MISTRG-6/Fah-ko mice are engrafted intrahepatically with fetal liver cells, followed by intrasplenical engraftment of the human adult hepatocytes together with the progenitors of human cholangiocytes. 12 wks post transplantation, single-cell RNA sequencing is performed that is run routinely to examine the actual degree of humanization of the different liver cell types in these mice. The single cell RNA-seq results are confirmed with Immunohistochemistry and FACS. Moreover, whether the engrafted human cells are functional is examined.

Section 2: Induce NAFLD in a Mouse with Human Liver

The mice of section 1 are treated with Western Diet (WD) to induce liver steatosis (NAFL), inflammation and fibrosis (NASH) in an obese and diabetic background. The extent to which this dietary approach recapitulates features of human NAFLD is determined. in this model, single cell RNA sequencing is performed to determine for the first time the molecules/pathways that are upregulated or downregulated in each human liver cell type in vivo at early stage (fatty liver without inflammation and fibrosis) and at late stage (fatty liver with inflammation and fibrosis) post WD treatment. This model is very important translational tool for future studies to examine efficacy of drugs.

MISTRG mice have been shown to support the growth of human innate and adaptive immune cells (Rongvaux et al., 2014, Nat Biotechnol., 32:364-372). In the liver of MISTRG-6 mice that were engrafted intrahepatically with fetal liver Hematopoietic stem cells (CD34+ cells) at neonatal age, the majority of liver immune cells (CD45+) were human (FIG. 67) as determined by FACS after liver digestion and exclusion of hepatocytes. CD45 is a common lymphocyte antigen that is expressed on almost all hematopoietic cells except for mature erythrocytes (Nakano et al., 1990, Acta Pathol Jpn., 40:107-115). 70% of liver CD45+ cells were the Kupffer Cells (CD68+) that were also found to be human in about 70-90% in the liver of MISTRG-6 mice. CD34+ cells that were engrafted in the liver of other immunodeficient mice have been shown to support the growth of liver endothelial cells (Fomin et al, 2017, Open Biol., 7; Fomin et al., 2013, PLoS One, 8: e77255) and to be the precursors of stellate cells (Suskind etl al., 2004, J Hepatoi., 40:261-268). Therefore, the expression of human markers was examined by FACS for liver endothelial cells, such as Vascular Adhesion Protein 1 (VAP-1), and for stellate cells, such as the desmin (Neubauer et al., 1996, J Hepatol., 24:719-730) in the liver cells of engrafted MISTRG-6 mice. Regarding the endothelial cells, it was found that in VAN+subpopulation 60-75% were human and the rest were mouse (FIG. 67). Since VAP-1+ cells consisted 70% of total liver endothelial cells, 50-60% of MISTRG-6 liver endotelial cells were expected to be human. Regarding the stellate cells, 60% of them were expected to express desmin (Niki et al., 1996, Hepatology, 23:1538-1545). It was found that in desmin+ stellate cells subpopulation, 75% of cells were human and 25% were mouse (FIG. 67). Therefore, in the total stellate cells, about 50-60% were expected to be human.

Development of a human liver with a high degree of human epithelial cells (cholangiocytes and hepatocytes) and human stromal cells (endothelial, stellate and immune cells) is expected. In the mouse with human liver, the treatment with WD is expected to induce fatty liver and NASH. With the single-cell RNA-Seq, it is expected to find for the first time what pathways are altered in each human cell type in the different stages of NAFLD (fatty liver or NASH).

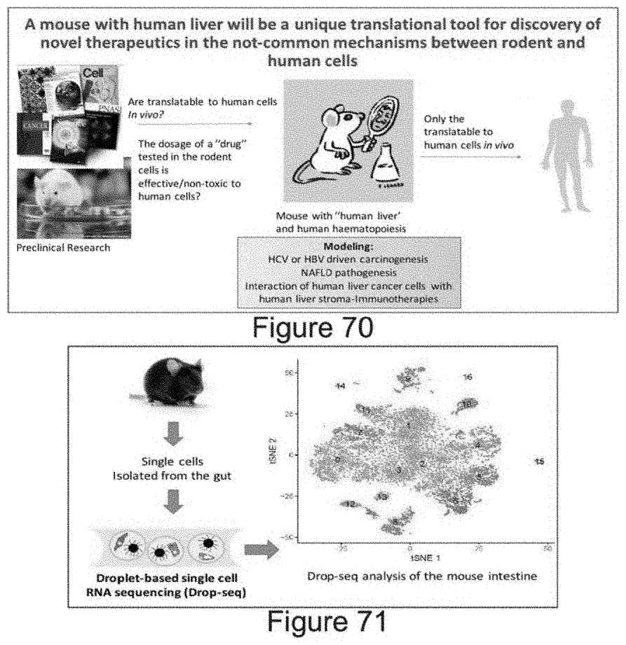

The popular murine/rodent studies of NAFLD have revealed several therapeutic targets for NAFLD. However, the extent to which these targets are translatable to human cells in vivo is not known. PPARa agonists that are currently in clinical trials in humans, activate different pathways in human cells than in rodent cells (Rakhshandehroo et 2009, PLoS One, 4: e6796). Moreover, there are many differences in pathogenic mechanisms that are known to drive NAFLD between human and rodent (FIG. 64). A humanized liver allows to study these different pathological mechanisms of liver damage and the function of human molecular targets in human liver cells within the pathophysiological context of a liver disease in vivo. These mice can be colonized with human inicrobiota derived from patients with NAFLD to study the cross-talk of liver-gut axis. In the future this model can be used: 1) to study the altered pathways in human liver cells that are able to drive HCC in NASH patients. 2) These mice can be infected with Hepatitis B Virus (HBV) or Hepatitis C Virus (HCV), which cannot infect rodent liver cells and can increase the risk of HCC by 50-100 fold (for HBV) and 20 fold (for HCV)(El-Serag, 2012, Gastroenterology, 142:1264-1273); such infected mice could be used to study the mechanisms of HCV or HBV driven HCC. 3) These mice can be used to examine mechanisms to increase the therapeutic effect of various combinations of immunotherapies targeted the human immune system against the Patient Derived. Liver Cancer Cells that can engrafted in the human liver, 4) to study the factors that are secreted by human liver cells and drives liver metastasis of non-liver tumors using Patient Derived Xenografts (PDX) models. 5) Finally, results from animal models can be validated in this model to examine if they are translatable to human cells (FIG. 70). Therefore, this mouse model is a very important technological advancement for basic and translational studies for novel therapeutics.

In summary, MISTRG-6/Fah-KO mice were generated and are available as colonies. Moreover, human cryopreserved hepatocytes and human fetal liver cells were made available. If the humanization of liver endothelial cells, hepatic stellate cells and cholangiocytes is not more than 70% then, together with the human hepatocytes, human adult liver endothelial cells, hepatic stellate cells and cholangiocytes are engrafted. Based upon the present data, the development of a humanized liver model is achievable. Further, a good characterization of the NAFLD is performed and reveals putative novel pathways with high translational potential.

The materials and methods employed in these experiments are now described.

### Study Design and Methodology for Example 6

The present data suggested that MISTRG-6 mice can support the growth of human immune cells and a subpopulation of human endothelial and human stellate cells. Since, the humanization of hepatocytes can be supported in the MSTRG/Fah-KO mice that exist as alive colony, this mouse is used to create a humanized liver having functional all the human liver cell types (immune cells, endothelial cells, hepatic stellate cell, cholangiocytes and hepatocytes). In section 1 the MISTRG/Fah-KO mice is engrafted with fetal liver cells (CD34+ or all the fetal liver cells) at 2 days postnatal (intrahepatic injection) and at 6 weeks post transplantation is injected intraperitoneally with Galactosamine D (GaIN-D) (700 mg/Kg), then 36 hours post GaiN-D treatment, is engrafted with human hepatic progenitor cells CD34+CD117+ isolated from fetal human liver together with human adult hepatocytes (FIG. 4). One day after engrafiment the NTBC water is discountinued gradually and completely withdrawn one week after transplantation. After withdrawal of NTBC water, the Fah-KO mouse hepatocytes die and then are replenished by transplanted human adult hepatocytes. Twelve weeks post hepatocytes transplantation these animals is expected to be highly engrafted (up to 90%) with human hepatocytes that are functional since they are able to secrete human albumin in the mouse plasma at levels comparable to human plasma (Azunia. et al., 2007, Nat Biotechnol., 25:903-910). To examine whether the engrafted human cholangiocytes is functional, human cholangiocytes is isolated to examine the bicarbonate secretion upon secretin treatment. Proliferation of human cholangiocytes is also examined after treatment with 0.1% 3,5-diethoxycarbonyl-11,4-dihydrocollidine (DDC) in their diet for 2 weeks. To examine whether the engrafted human liver endothelial are functional, the levels of human Factor VIII (FVIII) (that is produced by liver endothelial cells) are evaluated in the plasma by ELISA (Fomin et al., 2013, PLoS One, 8:e77255) and the uptake of FITC-labeled formaldehyde-treated serum albumin (FITC-FSA) as described by the laboratory of Bard Smedsrod (Oie et al., 2016, PLoS One, 11:e0160602). To examine whether the engrafted human stellate cells are functional in the MISTRG-Fah-KO mice, Carbon tetrachloride (CCl4) is administrated two times per week for 4 weeks and their activation (human aSMA and human collagen 1 expression) is examined. In section 2, the mice of Section 1 that have the highest degree of humanization of all liver cell types, are fed with a diet that mimics the dietary habits of people that develop NAFLD. Initially, gnotobiotic MISTRG-Fah-KO mice is colonized with microbiota from health or NAFLD patients. Then, 16-wks-old mice are treated with High-Fat/High Sucrose, Low Fiber diet with 1% Cholesterol, and high-fructose corn syrup equivalent in water (FIG. 69). This diet mimics the Western Diet and can induce liver steatosis and insulin resistance at 4 wks post treatment and at 16 wks post treatment fibrosis, inflammation, moderate obesity, hyperlipidemia as well as some of the metabolic alterations of T2D (hyperglycemia and hyperinsulinemia) (Surwit et al., 1988, Diabetes, 37: 1163-1167; Melts et al., 2015, J Nutr Biochem., 26:285-292), At 4 wks and at 16 wks post treatment the liver is analyzed for the degree of NAFLD. In these livers, single cell RNA-seq is performed that is described in the analytical methods to examine the pathways that are altered in each human cell type at fatty liver stage (4 weeks of WD treatment) and at NASH stage (16 weeks of WD treatment).

### Description of Analytical Methods for Example 6

Human albumin is measured in the blood by ELISA in the engrafted MISTRG-Fah-KO mice (2-5 mg/ml of human albumin in the blood is indicative for 70-90% hepatocytes humanization). The NAFLD activity score is examined by measuring lobular inflammation, steatosis, hepatocyte ballooning according to the criteria of Kleiner (Kleiner et al., 2005, Hepatology, 41:1313-1321), fibrosis score and collagen deposition (morphometric analysis of Sirius red staining), inflammation (FACS analysis of liver immune cells). The lipid profile is analyzed using High-performance Liquid Chromatography-Tandem Mass Spectrometry (LC-MS-MS) with the Instruments available in the Yale Analytical Core and by employing methods that was previously optimized and applied (Katie, E et al., 2017, Hepatology, 65:1369-1383). The Single cell transcriptomics (Single-Cell RNA-seq) is performed as previously described (Macosko et al., 2015, Cell, 161:1202-1214; Shekhar et al., 2016, Cell, 166:1308-1323 e30) (FIG. 71). Single cell RNA-seq is a high-throughput and relatively inexpensive single cell RNA-seq platform that can be implemented with simple lab instrumentation and can effectively separates mouse vs human cells. This assay, besides the percentage of human vs mouse cells can show the genes that are upregulated or downregulated in each cell type.

The data described herein demonstrate that the genetically modified non-human animals described herein represent a novel in vivo animal model of human hematopoiesis, as well as for human diseases or disorders, such as sickle cell disease, MDS, liver diseases, leukemia, and melanoma.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A genetically modified non-human animal comprising:
a) a genome comprising a nucleic acid encoding at least one of the group consisting of human M-CSF, human IL-3, human GM-CSF, human SIRPA, and human TPO, wherein the nucleic acid is operably linked to a promoter; and
b) a nucleic acid encoding at least one of the group consisting of cKit or a mutant thereof, fumarylacetoacetate hydrolase (Fah) or a mutant thereof, and any combination thereof;
wherein the animal expresses at least one polypeptide selected from the group consisting of human M-CSF, human IL-3, human GM-CSF, human SIRPA, and human TPO;
wherein the animal does not express Fah (Fah^{-/-}); and
wherein the animal comprises cKitw41 mutation or cKitWV mutation.

2. The genetically modified non-human animal of claim 1, wherein the animal comprises a genome comprising a nucleic acid encoding human M-CSF, human IL-3, human GM-CSF, human SIRPA, and human TPO, wherein each of the nucleic acids encoding human M-CSF, human IL-3, human GM-CSF, human SIRPA, and human TPO is operably linked to a promoter, and
wherein the animal expresses human M-CSF polypeptide, human IL-3 polypeptide, human GM-CSF polypeptide, human SIRPA polypeptide, and human TPO polypeptide.

3. The genetically modified non-human animal of claim 1, wherein the animal is immunodeficient.

4. The genetically modified non-human animal of claim 1, wherein the animal further comprises a genome comprising a nucleic acid encoding human IL-6.

5. The genetically modified non-human animal of claim 1, wherein the animal expresses human IL-6.

6. The genetically modified non-human animal of claim 1, wherein the animal does not express recombination activating gene 2 (Rag-2^{-/-}).

7. The genetically modified non-human animal of claim 1, wherein the animal does not express IL2 receptor gamma chain (gamma chain^{-/-}).

8. The genetically modified non-human animal of claim 1, wherein the animal does not express Rag-2; and wherein the animal does not express IL2 receptor gamma chain (Rag-2^{-/-} gamma chain^{-/-}).

9. The genetically modified non-human animal of claim 1, wherein the animal does not express Rag-2, wherein the animal does not express IL2 receptor gamma chain, wherein the animal does not express Fah, and wherein the animal comprises cKitw41 mutation (Rag-2^{-/-}gamma chain^{-/-} Fah^{-/-} cKit^{w41/w41}) or cKitWV mutation (Rag-2^{-/-} gamma chain^{-/-} Fah^{-/-} cKitWV).

10. The genetically modified non-human animal of claim 1, wherein the animal is a rodent.

11. (canceled)

12. The genetically modified non-human animal of claim 1, further comprising human hematopoietic cells.

13. The genetically modified non-human animal of claim 1, further comprising a human cancer cell.

14. (canceled)

15. The genetically modified non-human animal of claim 1, further comprising a human liver cell.

16. The genetically modified non-human animal of claim 1, further comprising a human spleen cell.

17. The genetically modified non-human animal of claim 1, wherein the animal has a sickle cell disease.

18. The genetically modified non-human animal of claim 1, wherein the animal is infected with malaria or hepatitis.

19. The genetically modified non-human animal of claim 1, wherein the animal has a liver disease.

20. (canceled)

21. A method of hematopoietic stem and progenitor cell (HSPC) engraftment or human erythropoiesis in a genetically modified non-human animal, the method comprising the step of:
administering at least one HSPCs to the genetically modified animal expressing at least one of the group consisting of human M-CSF, human IL-3, human GM-CSF, human SIRPA, and human TPO;
wherein the animal comprises a nucleic acid encoding at least one of the group consisting of cKit or a mutant thereof, fumarylacetoacetate hydrolase (Fah) or a mutant thereof, and any combination thereof;
wherein the animal expresses at least one of the group consisting of human M-CSF, human IL-3, human GM-CSF, human SIRPA, and human TPO,
wherein the animal does not express Fah (Fah^{-/-}); and
wherein the animal comprises cKitw41 mutation or cKitWV mutation.

22.-37. (canceled)

38. A genetically modified Rag-2^{-/-}, gamma chain^{-/-}, Fah^{-/-}, cKit^{w41/w41} non-human animal having a genome comprising a nucleic acid encoding at least one of the group consisting of human M-CSF, human IL-3, human GM-CSF, human SIRPA, and human TPO, operably linked to a promoter, wherein the mouse expresses at least one polypeptide selected from the group consisting of human M-CSF, human IL-3, human GM-CSF, human SIRPA, and human TPO.

39.-50. (canceled)

51. The genetically modified non-human animal of claim 1, wherein the animal does not express SRB1 (SRB1^{-/-}), SRB2 (SRB2^{-/-}), or a combination thereof (SRB1^{-/-} SRB2^{-/-}).

52.-53. (canceled)
